SEQUENCE LISTING (67 sequences)

SEQ ID NO: 1 - DNA, 2151 bp, Homo sapiens
  source 1..2151: mol_type = genomic DNA
atggccacctcccaccgagtggcgaagctggtggcctccagtctccagaccccggtaaatcccatcactggagcgcgggtcgcccagtacgaacgcgaagaccccttaaaggccctggcggcagcggaggcgatcttggaggacgaagaggaggagaaagtggcgcagcccgctggggcatcggctgatttgaacaccagcttttctggggtggatgaacatgcaccgataagctatgaggactttgtgaactttcctgatattcaccactctaatgaggagtatttcaagaaagtagaagagttgaaggctgcccacatagaaactatggcaaaattagagaaaatgtaccaggataaattacatttaaaggaagttcagccagtggtcatcagagaagactctcttagtgactcttccagatctgtatcagaaaagaactcctatcaccctgtctcattaatgacatcattttcagagcctgatttaggccagtcttcctccttgtatgtgtcctcctctgaagaggagttacccaacctagaaaaagagtatcctaggaaaaacagaatgatgacctatgctaaggagctcatcaacaatatgtggacagacttttgtgttgaggattatattcgctgtaaagatactggcttccatgcagctgaaaaaagaaggaagaaacgaaaagaatgggtgcccacaattacagtaccggagccttttcaaatgatgataagagaacagaagaaaaaagaagagtccatgaaatctaaatcagatatcgaaatggtacataaagcgctcaaaaaacaagaagaggatccagagtataagaagaaattccgagccaatccagttcctgcatctgtctttctccccctttaccatgatttagtcaagcaaaaagaagaacggagaaggtctctgaaggagaaaagcaaagaagctcttttggcctcacaaaagccatttaaatttatagcaagggaggaacagaagcgagcagcccgggaaaagcagctgagagactttcttaagtataaaaagaaaacaaatcgatttaaagccagacccattcctcgatctacttatggttcaactaccaatgacaagttaaaagaagaagagctctatcgaaaccttaggacacagctgagagcccaggagcatttacagaactcatctcctctgccttgtaggtcagcttgtggatgcaggaaccccaggtgtcctgaacaggctgtaaagttgaagtgtaaacacaaggttaggtgcccaactcctgattttgaggaccttcctgagagataccagaaacacctctcagaacacaagtctccaaaactcttaacagtgtgtaaaccatttgatcttcatgcatctccacatgcatctattaaaagagaaaaaattttggcagacatcgaagcagatgaagaaaatttaaaagaaacacgttggccttatttgtctccaaggcgtaagtcaccagtaagatgtgcaggtgtaaaccctgtgccttgtaactgcaaccctcccgtgcccacggtatcttccagaggacgagaacaagccgtaaggagatcacttgaggaaaagaaaatgttggaagaagagagaaatcggatcctaactaaacagaagcaaagaatgaaagaattgcagaaactcctgacaacccgggctaaggcttatgactcacatcaaagtttagctcaaatatctaaatccagagtaaaatgtctcagaaagagcgaaaaggaaaggatgagagaataccaacgagaactagaagaaagagaagaaaaattaaaaaagaggccactgctatttgaaagagttgctcagaaaaatgcaagaatggcagcagaaaagcattattctaataccctaaaagcactaggaatatctgatgagtttgtttcaaagaaaggccaaagtggaaaagtacttgagtacttcaacaatcaagagacgaaaagtgtcactgaagacaaagaaagctttaatgaagaagaaaaaatagaagaaagagagaatggggaagaaaattattttattgataccaacagccaggattcttacaaggaaaaagatgaagccaatgaggaaagtgaagaagagaaatctgttgaagaatcacactga

SEQ ID NO: 2 - DNA, 1970 bp, Homo sapiens
  source 1..1970: mol_type = genomic DNA
atggccacctcccaccgagtggcgaagctggtggcctccagtctccagaccccggtaaatcccatcactggagcgcgggtcgcccagtacgaacgcgaagaccccttaaaggccctggcggcagcggaggcgatcttggaggacgaagaggaggagaaagtggcgcagcccgctggggcatcggctgatttgaacaccagcttttctggggtggatgaacatgcaccgataagctatgaggactttgtgaactttcctgatattcaccactctaatgaggagtatttcaagaaagtagaagagttgaaggctgcccacatagaaactatggcaaaattagagaaaatgtaccaggataaattacatttaaaggaagttcagccagtggtcatcagagaagactctcttagtgactcttccagatctgtatcagaaaagaactcctatcaccctgtctcattaatgacatcattttcagagcctgatttaggccagtcttcctccttgtatgtgtcctcctctgaagaggagttacccaacctagaaaaagagtatcctaggaaaaacagaatgatgacctatgctaaggagctcatcaacaatatgtggacagacttttgtgttgaggattatattcgctgtaaagatactggcttccatgcagctgaaaaaagaaggaagaaacgaaaagaatgggtgcccacaattacagtaccggagccttttcaaatgatgataagagaacagaagaaaaaagaagagtccatgaaatctaaatcagatatcgaaatggtacataaagcgctcaaaaaacaagaagaggatccagagtataagaagaaattccgagccaatccagttcctgcatctgtctttctccccctttaccatgatttagtcaagcaaaaagaagaacggagaaggtctctgaaggagaaaagcaaagaagctcttttggcctcacaaaagccatttaaatttatagcaagggaggaacagaagcgagcagcccgggaaaagcagctgagagactttcttaagtataaaaagaaaacaaatcgatttaaagccagacccattcctcgatctacttatggttcaactaccaatgacaagttaaaagaagaagagctctatcgaaaccttaggacacagctgagagcccaggagcatttacagaactcatctcctctgccttgtaggtcagcttgcggatgcaggaaccccaggtgtcctgaacaggctgtaaagttgaagtgtaaacacaaggttaggtgcccaactcctgattttgaggaccttcctgagagataccagaaacacctctcagaacacaagtctccaaaactcttaacagtgtgtaaaccatttgatcttcatgcatctccacatgcatctattaaaagagaaaaaattttggcagacatcgaagcagatgaagaaaatttaaaagaaacacgttggccttatttgtctccaaggcgtaagtcaccagtaagatgtgcaggtgtaaaccctgtgccttgtaactgcaaccctcccgtgcccacggtatcttccagaggacgagaacaagccgtaagaaagagcgaaaaggaaaggatgagagaataccaacgagaactagaagaaagagaagaaaaattaaaaaagaggccactgctatttgaaagagttgctcagaaaaatgcaagaatggcagcagaaaagcattattctaataccctaaaagcactaggaatatctgatgagtttgtttcaaagaaaggccaaagtggaaaagtacttgagtacttcaacaatcaagagacgaaaagtgtcactgaagacaaagaaagctttaatgaagaagaaaaaatagaagaaagagagaatggggaagaaaattattttattgataccaacagccaggattcttacaaggaaaaagatgaagccaatgaggaaagtgaagaagagaaatctgttga

SEQ ID NO: 3 - DNA, 1863 bp, Mus musculus
  source 1..1863: mol_type = genomic DNA
atgaacttctctgatatttaccactctgatgaagagtatttcaggaaactcaaagacctgaaggctgtccatgaagaaaccatgtcaaaattagagaaaatgtaccaggacaagctaaatataaaagacatccaggcggggttcatcagggatgggatcagcgattcgtcttcgagttctgcatcggaaaagagctgctcacatcctgccttgtcagtgacctcactttcagagcctgacctagacgggtcgtcctccttgtccacaactactgatgaggggttgcccgacctagaggaaaagactcccggggaaagcagcgcaatggtccatgcccaggagctcataaacaacatgtggaacgacttttctgttgaagattatattcagtatgactcagactcccgaacagctaagaaaaaaaggaagaaagctaaatccttgacacccaagattacagtgcctgtaccctttgaaatgacggtaagagagcagaatcggagagagaaggccttgagtgctcggtcagatctggaaacaaagctgctcaagagggacgaggacgatgcagagtgtaagaagaaattccgagccaatcccgttccttcctgcgtgctccttcccctttatgaggatctagtcaagcagagtgaggagcgccggaagaaggcccgggagaggaacagagctgcgctcctggcctccctgaagccctttaagttcattgcgagggaggaacagaagcaagcagtccgcgagaagaagctgagagacctttttagggctaaaaggaaaacgaatcagtttaaagccaaacctgtgcctcgttttatttacaggccagctgcgagtgacaagccaaaggaagaagagctctatggggacagcaggatgctgccaaaggtcagagatctgttacagaattcgccttggcccagtcggtcagcttgcagacgtttcagggaccccagaagtcctgcaaagcccaggggtaaacacaggcgtaggtgcctgaggcgtgatggagacttagaaaaatggaaggagcccttctctgaatacagctttctgaaatgcccaatgctttgtgaagagtgttgtcttcacgaatccccttgtgactctgataaaagacagaaactcttggcagacatcagagcagatgaagaaattttgagagaaacacgcaggccgggtcggtctcctaggcgcaagtcaccgggaagaagctcgaacccaaagcccaggcctcatgaatgcagcccaccgatgcccaccgcgtcttcccgagggcgggagcaagccatcaggaaatcccttgaggaaaagaaaatgttggaagaagagagaaatcggatcctaactaaacagaagcaaagaatgaaagacttgcagaaactgctggcaacccgggttaaggcttatggctcccaccagagcttgtctcagatagttaaagccagggtgaaggagctcagaaggagtgaaaaggccaggatgagagaatattggcaagaactggaggaacaagaggaaaaactgcagaagaggccgatgttgtttgaaagagtcacccagagaaatgcaaggatggccgcagagaggtgttactctaacactctaaaagcactaggactccttgaggagtttgtttcagagaaaggccaaagtggaaaagtgtctgaagacttcaccaggcaagagctgagaagctgcacactggagaaagaaagctcttatggagaagaaagagagaatgaggaagaaagctatttaactgatatcagcagccaggattcttgcaagggaaacaaggaagatattaaagatattaaaagtggagaagaaaattctggagaatga

SEQ ID NO: 4 - DNA, 1695 bp, Mus musculus
  source 1..1695: mol_type = genomic DNA
atgaacttctctgatatttaccactctgatgaagagtatttcaggaaactcaaagacctgaaggctgtccatgaagaaaccatgtcaaaattagagaaaatgtaccaggacaagctaaatataaaagacatccaggcggggttcatcagggatgggatcagcgattcgtcttcgagttctgcatcggaaaagagctgctcacatcctgccttgtcagtgacctcactttcagagcctgacctagacgggtcgtcctccttgtccacaactactgatgaggggttgcccgacctagaggaaaagactcccggggaaagcagcgcaatggtccatgcccaggagctcataaacaacatgtggaacgacttttctgttgaagattatattcagtatgactcagactcccgaacagctaagaaaaaaaggaagaaagctaaatccttgacacccaagattacagtgcctgtaccctttgaaatgacggtaagagagcagaatcggagagagaaggccttgagtgctcggtcagatctggaaacaaagctgctcaagagggacgaggacgatgcagagtgtaagaagaaattccgagccaatcccgttccttcctgcgtgctccttcccctttatgaggatctagtcaagcagagtgaggagcgccggaagaaggcccgggagaggaacagagctgcgctcctggcctccctgaagccctttaagttcattgcgagggaggaacagaagcaagcagtccgcgagaagaagctgagagacctttttagggctaaaaggaaaacgaatcagtttaaagccaaacctgtgcctcgttttatttacaggccagctgcgagtgacaagccaaaggaagaagagctctatggggacagcaggatgctgccaaaggtcagagatctgttacagaattcgccttggcccagtcggtcagcttgcagacgtttcagggaccccagaagtcctgcaaagcccaggggtaaacacaggcgtaggtgcctgaggcgtgatggagacttagaaaaatggaaggagcccttctctgaatacagctttctgaaatgcccaatgctttgtgaagagtgttgtcttcacgaatccccttgtgactctgataaaagacagaaactcttggcagacatcagagcagatgaagaaattttgagagaaacacgcaggccgggtcggtctcctaggcgcaagtcaccgggaagaagctcgaacccaaagcccaggcctcatgaatgcagcccaccgatgcccaccgcgtcttcccgagggcgggagcaagccatcagaaggagtgaaaaggccaggatgagagaatattggcaagaactggaggaacaagaggaaaaactgcagaagaggccgatgttgtttgaaagagtcacccagagaaatgcaaggatggccgcagagaggtgttactctaacactctaaaagcactaggactccttgaggagtttgtttcagagaaaggccaaagtggaaaagtgtctgaagacttcaccaggcaagagctgagaagctgcacactggagaaagaaagctcttatggagaagaaagagagaatgaggaagaaagctatttaactgatatcagcagccaggattcttgcaagggaaacaaggaagatattaaagatattaaaagtggagaagaaaattctggagaatga

SEQ ID NO: 5 - DNA, 1596 bp, Mus musculus
  source 1..1596: mol_type = genomic DNA
atgaacttctctgatatttaccactctgatgaagagtatttcaggaaactcaaagacctgaaggctgtccatgaagaaaccatgtcaaaattagagaaaatgtaccaggacaagctaaatataaaagacatccaggcggggttcatcagggatgggatcagcgattcgtcttcgagttctgcatcggaaaagagctgctcacatcctgccttgtcagtgacctcactttcagagcctgacctagacgggtcgtcctccttgtccacaactactgatgaggggttgcccgacctagaggaaaagactcccggggaaagcagcgcaatggtccatgcccaggagctcataaacaacatgtggaacgacttttctgttgaagattatattcagtatgactcagactcccgaacagctaagaaaaaaaggaagaaagctaaatccttgacacccaagattacagtgcctgtaccctttgaaatgacggtaagagagcagaatcggagagagaaggccttgagtgctcggtcagatctggaaacaaagctgctcaagagggacgaggacgatgcagagtgtaagaagaaattccgagccaatcccgttccttcctgcgtgctccttcccctttatgaggatctagtcaagcagagtgaggagcgccggaagaaggcccgggagaggaacagagctgcgctcctggcctccctgaagccctttaagttcattgcgagggaggaacagaagcaagcagtccgcgagaagaagctgagagacctttttagggctaaaaggaaaacgaatcagtttaaagccaaacctgtgcctcgttttatttacaggccagctgcgagtgacaagccaaaggaagaagagctctatggggacagcaggatgctgccaaaggtcagagatctgttacagaattcgccttggcccagtcggtcagcttgcagacgtttcagggaccccagaagtcctgcaaagcccaggggtaaacacaggcgtaggtgcctgaggcgtgatggagacttagaaaaatggaaggagcccttctctgaatacagctttctgaaatgcccaatgctttgtgaagagtgttgtcttcacgaatccccttgtgactctgataaaagacagaaactcttggcagacatcagagcagatgaagaaattttgagagaaacacgcaggccgggtcggtctcctaggcgcaagtcaccgggaagaagctcgaacccaaagcccaggcctcatgaatgcagcccaccgatgcccaccgcgtcttcccgagggcgggagcaagccatcaggaaatcccttgaggaaaagaaaatgttggaagaagagagaaatcggatcctaactaaacagaagcaaagaatgaaagacttgcagaaactgctggcaacccgggttaaggcttatggctcccaccagagcttgtctcagatagttaaagccagggtgaaggagctcagaaggagtgaaaaggccaggatgagagaatattggcaagaactggaggaacaagaggaaaaactgcagaagaggccgatgttgtttgaaagagtcacccaggttgtgtttattggttag

SEQ ID NO: 6 - DNA, 1428 bp, Mus musculus
  source 1..1428: mol_type = genomic DNA
atgaacttctctgatatttaccactctgatgaagagtatttcaggaaactcaaagacctgaaggctgtccatgaagaaaccatgtcaaaattagagaaaatgtaccaggacaagctaaatataaaagacatccaggcggggttcatcagggatgggatcagcgattcgtcttcgagttctgcatcggaaaagagctgctcacatcctgccttgtcagtgacctcactttcagagcctgacctagacgggtcgtcctccttgtccacaactactgatgaggggttgcccgacctagaggaaaagactcccggggaaagcagcgcaatggtccatgcccaggagctcataaacaacatgtggaacgacttttctgttgaagattatattcagtatgactcagactcccgaacagctaagaaaaaaaggaagaaagctaaatccttgacacccaagattacagtgcctgtaccctttgaaatgacggtaagagagcagaatcggagagagaaggccttgagtgctcggtcagatctggaaacaaagctgctcaagagggacgaggacgatgcagagtgtaagaagaaattccgagccaatcccgttccttcctgcgtgctccttcccctttatgaggatctagtcaagcagagtgaggagcgccggaagaaggcccgggagaggaacagagctgcgctcctggcctccctgaagccctttaagttcattgcgagggaggaacagaagcaagcagtccgcgagaagaagctgagagacctttttagggctaaaaggaaaacgaatcagtttaaagccaaacctgtgcctcgttttatttacaggccagctgcgagtgacaagccaaaggaagaagagctctatggggacagcaggatgctgccaaaggtcagagatctgttacagaattcgccttggcccagtcggtcagcttgcagacgtttcagggaccccagaagtcctgcaaagcccaggggtaaacacaggcgtaggtgcctgaggcgtgatggagacttagaaaaatggaaggagcccttctctgaatacagctttctgaaatgcccaatgctttgtgaagagtgttgtcttcacgaatccccttgtgactctgataaaagacagaaactcttggcagacatcagagcagatgaagaaattttgagagaaacacgcaggccgggtcggtctcctaggcgcaagtcaccgggaagaagctcgaacccaaagcccaggcctcatgaatgcagcccaccgatgcccaccgcgtcttcccgagggcgggagcaagccatcagaaggagtgaaaaggccaggatgagagaatattggcaagaactggaggaacaagaggaaaaactgcagaagaggccgatgttgtttgaaagagtcacccaggttgtgtttattggttag

SEQ ID NO: 7 - DNA, 608 bp, synthetic construct
  source 1..608: mol_type = other DNA
gggctcagaccttagagacgggttctcgacgatgtgcagcgcataacgaggaaaatactgaccggagggggccggtcgtagggctggggagcaggaggagggcaaaggatagagagggggtagatagggagtagatagggggcggcagcctcggcctcagtaagcaggggctcagggagcagatgtctgagactgcaccacgtccgctattttgaggtttggcttctcttactgccacgtggtgcagagggaaggcgccatttgatgcgtccaaatcccgagtgggaagagaagatacggatgagggagatggatgtgtctgtggtgactcttgggcacttgtgccgttcaatcctgtcactctcaagctgaagaacccaaagcaaacgccaggtttacaatacctgcactatcgctcattagtgggagggtcaggcgccacccaactctgggcttttattggtcatgtcaagaaaaaagcgagagggacaactgggagcctccttctgattggtggattcctgctagccctcaagtcaatcagagtagaatgtaccgccctccttcccactctggcccccgcgggagacatgttaggtagcgggacg

SEQ ID NO: 8 - DNA, 457 bp, synthetic construct
  source 1..457: mol_type = other DNA
ctctctcattctttacaaattcagcgaaaaaaattttttcaaaaattacaggttgcagatgaaactatataggctgcagcacaaataattcttcataggtgctgattactaataaagcaacaggtgctggaaggattcattatctaaatgggtcaagccaatctagggaagattatcctctaatctggtggctgcatttacaacattccacaaaaagccgattaaagggagaggatgctattttaggctcttggaaaaataatgtctatggcctaggttattttgtatactaaatattttctcagtggagacaattaaagattaaattagaattggaggtcatttactagccattcatctgttttaggttaaacactgtgagatcagtttatctgaaaagtttcggctagcgttaacagtattaagctgaaaacagaatggatccagtgcaaaatca

SEQ ID NO: 9 - DNA, 460 bp, synthetic construct
  source 1..460: mol_type = other DNA
ccaaccttgtcactcttctgagaccgccacccagtaacacgccctgctttcctccgccgccgccgtctcctccatcaaccgtctccttctaacatgttattgcagttaataaaaaagcgttcttttcttcacaaaagcagttgtttcattgtgtaatgtgagaagtctgcgttcaggcggttagacgcaaaggagtggaataagtgccctgttcagcctgccaaccttcggaaccagccaacagactcggtactgactgccatgatccagtaagcacccgactcgggtctctggcagcctgagtggattttgcgtgacttttggtccctggcggctcctgtagcgtccccagttacggcgcccatagcaaccggctccctagctaggcgcccccgggttgccaggggccgcaccagctttcccgccccgggccagcgcaggcgctcaggcctcggaggcg

SEQ ID NO: 10 - DNA, 1068 bp, synthetic construct
  source 1..1068: mol_type = other DNA
gggctcagaccttagagacgggttctcgacgatgtgcagcgcataacgaggaaaatactgaccggagggggccggtcgtagggctggggagcaggaggagggcaaaggatagagagggggtagatagggagtagatagggggcggcagcctcggcctcagtaagcaggggctcagggagcagatgtctgagactgcaccacgtccgctattttgaggtttggcttctcttactgccacgtggtgcagagggaaggcgccatttgatgcgtccaaatcccgagtgggaagagaagatacggatgagggagatggatgtgtctgtggtgactcttgggcacttgtgccgttcaatcctgtcactctcaagctgaagaacccaaagcaaacgccaggtttacaatacctgcactatcgctcattagtgggagggtcaggcgccacccaactctgggcttttattggtcatgtcaagaaaaaagcgagagggacaactgggagcctccttctgattggtggattcctgctagccctcaagtcaatcagagtagaatgtaccgccctccttcccactctggcccccgcgggagacatgttaggtagcgggacgccaaccttgtcactcttctgagaccgccacccagtaacacgccctgctttcctccgccgccgccgtctcctccatcaaccgtctccttctaacatgttattgcagttaataaaaaagcgttcttttcttcacaaaagcagttgtttcattgtgtaatgtgagaagtctgcgttcaggcggttagacgcaaaggagtggaataagtgccctgttcagcctgccaaccttcggaaccagccaacagactcggtactgactgccatgatccagtaagcacccgactcgggtctctggcagcctgagtggattttgcgtgacttttggtccctggcggctcctgtagcgtccccagttacggcgcccatagcaaccggctccctagctaggcgcccccgggttgccaggggccgcaccagctttcccgccccgggccagcgcaggcgctcaggcctcggaggcg

SEQ ID NO: 11 - DNA, 917 bp, synthetic construct
  source 1..917: mol_type = other DNA
ctctctcattctttacaaattcagcgaaaaaaattttttcaaaaattacaggttgcagatgaaactatataggctgcagcacaaataattcttcataggtgctgattactaataaagcaacaggtgctggaaggattcattatctaaatgggtcaagccaatctagggaagattatcctctaatctggtggctgcatttacaacattccacaaaaagccgattaaagggagaggatgctattttaggctcttggaaaaataatgtctatggcctaggttattttgtatactaaatattttctcagtggagacaattaaagattaaattagaattggaggtcatttactagccattcatctgttttaggttaaacactgtgagatcagtttatctgaaaagtttcggctagcgttaacagtattaagctgaaaacagaatggatccagtgcaaaatcaccaaccttgtcactcttctgagaccgccacccagtaacacgccctgctttcctccgccgccgccgtctcctccatcaaccgtctccttctaacatgttattgcagttaataaaaaagcgttcttttcttcacaaaagcagttgtttcattgtgtaatgtgagaagtctgcgttcaggcggttagacgcaaaggagtggaataagtgccctgttcagcctgccaaccttcggaaccagccaacagactcggtactgactgccatgatccagtaagcacccgactcgggtctctggcagcctgagtggattttgcgtgacttttggtccctggcggctcctgtagcgtccccagttacggcgcccatagcaaccggctccctagctaggcgcccccgggttgccaggggccgcaccagctttcccgccccgggccagcgcaggcgctcaggcctcggaggcg

SEQ ID NO: 12 - DNA, 241 bp, synthetic construct
  source 1..241: mol_type = other DNA
tggaggcagaggagaaggcccaggcttcccagcagggctaaggatatgcaaggagtgcattcatccggaggtgttggcagcatcccagccccaccccattctcatcgtaaatcaggctcacttccattggctgcatacggtggagtgatgtgaccatatgtcacttgagcattacacaaatcctaatgagctaaaaatatgtttgttttagctaattgacctctttggccttcataaagca

SEQ ID NO: 13 - DNA, 145 bp, synthetic construct
  source 1..145: mol_type = other DNA
ctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggca

SEQ ID NO: 14 - DNA, 566 bp, synthetic construct
  source 1..566: mol_type = other DNA
tggaggcagaggagaaggcccaggcttcccagcagggctaaggatatgcaaggagtgcattcatccggaggtgttggcagcatcccagccccaccccattctcatcgtaaatcaggctcacttccattggctgcatacggtggagtgatgtgaccatatgtcacttgagcattacacaaatcctaatgagctaaaaatatgtttgttttagctaattgacctctttggccttcataaagcaatcactagtgaattcgcggccgcctgcaggggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggca

SEQ ID NO: 15 - DNA, 595 bp, synthetic construct
  source 1..595: mol_type = other DNA
ggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaag

SEQ ID NO: 16 - DNA, 4044 bp, synthetic construct
  source 1..4044: mol_type = other DNA
gcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccgggccccccctcgaggtcgacggtatcgataagcttgatatcgaattatcacaagtttgtacaaaaaagcaggcttatggaggcagaggagaaggcccaggcttcccagcagggctaaggatatgcaaggagtgcattcatccggaggtgttggcagcatcccagccccaccccattctcatcgtaaatcaggctcacttccattggctgcatacggtggagtgatgtgaccatatgtcacttgagcattacacaaatcctaatgagctaaaaatatgtttgttttagctaattgacctctttggccttcataaagcaatcactagtgaattcgcggccgcctgcaggggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggcacaactttgtatacaaaagttgtatcggaggcggggcgatggccacctcccaccgagtggcgaagctggtggcctccagtctccagaccccggtaaatcccatcactggagcgcgggtcgcccagtacgaacgcgaagaccccttaaaggccctggcggcagcggaggcgatcttggaggacgaagaggaggagaaagtggcgcagcccgctggggcatcggctgatttgaacaccagcttttctggggtggatgaacatgcaccgataagctatgaggactttgtgaactttcctgatattcaccactctaatgaggagtatttcaagaaagtagaagagttgaaggctgcccacatagaaactatggcaaaattagagaaaatgtaccaggataaattacatttaaaggaagttcagccagtggtcatcagagaagactctcttagtgactcttccagatctgtatcagaaaagaactcctatcaccctgtctcattaatgacatcattttcagagcctgatttaggccagtcttcctccttgtatgtgtcctcctctgaagaggagttacccaacctagaaaaagagtatcctaggaaaaacagaatgatgacctatgctaaggagctcatcaacaatatgtggacagacttttgtgttgaggattatattcgctgtaaagatactggcttccatgcagctgaaaaaagaaggaagaaacgaaaagaatgggtgcccacaattacagtaccggagccttttcaaatgatgataagagaacagaagaaaaaagaagagtccatgaaatctaaatcagatatcgaaatggtacataaagcgctcaaaaaacaagaagaggatccagagtataagaagaaattccgagccaatccagttcctgcatctgtctttctccccctttaccatgatttagtcaagcaaaaagaagaacggagaaggtctctgaaggagaaaagcaaagaagctcttttggcctcacaaaagccatttaaatttatagcaagggaggaacagaagcgagcagcccgggaaaagcagctgagagactttcttaagtataaaaagaaaacaaatcgatttaaagccagacccattcctcgatctacttatggttcaactaccaatgacaagttaaaagaagaagagctctatcgaaaccttaggacacagctgagagcccaggagcatttacagaactcatctcctctgccttgtaggtcagcttgtggatgcaggaaccccaggtgtcctgaacaggctgtaaagttgaagtgtaaacacaaggttaggtgcccaactcctgattttgaggaccttcctgagagataccagaaacacctctcagaacacaagtctccaaaactcttaacagtgtgtaaaccatttgatcttcatgcatctccacatgcatctattaaaagagaaaaaattttggcagacatcgaagcagatgaagaaaatttaaaagaaacacgttggccttatttgtctccaaggcgtaagtcaccagtaagatgtgcaggtgtaaaccctgtgccttgtaactgcaaccctcccgtgcccacggtatcttccagaggacgagaacaagccgtaaggagatcacttgaggaaaagaaaatgttggaagaagagagaaatcggatcctaactaaacagaagcaaagaatgaaagaattgcagaaactcctgacaacccgggctaaggcttatgactcacatcaaagtttagctcaaatatctaaatccagagtaaaatgtctcagaaagagcgaaaaggaaaggatgagagaataccaacgagaactagaagaaagagaagaaaaattaaaaaagaggccactgctatttgaaagagttgctcagaaaaatgcaagaatggcagcagaaaagcattattctaataccctaaaagcactaggaatatctgatgagtttgtttcaaagaaaggccaaagtggaaaagtacttgagtacttcaacaatcaagagacgaaaagtgtcactgaagacaaagaaagctttaatgaagaagaaaaaatagaagaaagagagaatggggaagaaaattattttattgataccaacagccaggattcttacaaggaaaaagatgaagccaatgaggaaagtgaagaagagaaatctgttgaagaatcacactgataaaatcactagtgaattcgcggccgcctgcaggtcgacggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagaacccagctttcttgtacaaagtggtataattcctgcagcccgggggatccactagttctagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgc

SEQ ID NO: 17 - DNA, 3875 bp, synthetic construct
  source 1..3875: mol_type = other DNA
gcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccgggccccccctcgaggtcgacggtatcgataagcttgatatcgaattatcacaagtttgtacaaaaaagcaggcttatggaggcagaggagaaggcccaggcttcccagcagggctaaggatatgcaaggagtgcattcatccggaggtgttggcagcatcccagccccaccccattctcatcgtaaatcaggctcacttccattggctgcatacggtggagtgatgtgaccatatgtcacttgagcattacacaaatcctaatgagctaaaaatatgtttgttttagctaattgacctctttggccttcataaagcaatcactagtgaattcgcggccgcctgcaggggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggcacaactttgtatacaaaagttgtatcggaggcggggcgatggccacctcccaccgagtggcgaagctggtggcctccagtctccagaccccggtaaatcccatcactggagcgcgggtcgcccagtacgaacgcgaagaccccttaaaggccctggcggcagcggaggcgatcttggaggacgaagaggaggagaaagtggcgcagcccgctggggcatcggctgatttgaacaccagcttttctggggtggatgaacatgcaccgataagctatgaggactttgtgaactttcctgatattcaccactctaatgaggagtatttcaagaaagtagaagagttgaaggctgcccacatagaaactatggcaaaattagagaaaatgtaccaggataaattacatttaaaggaagttcagccagtggtcatcagagaagactctcttagtgactcttccagatctgtatcagaaaagaactcctatcaccctgtctcattaatgacatcattttcagagcctgatttaggccagtcttcctccttgtatgtgtcctcctctgaagaggagttacccaacctagaaaaagagtatcctaggaaaaacagaatgatgacctatgctaaggagctcatcaacaatatgtggacagacttttgtgttgaggattatattcgctgtaaagatactggcttccatgcagctgaaaaaagaaggaagaaacgaaaagaatgggtgcccacaattacagtaccggagccttttcaaatgatgataagagaacagaagaaaaaagaagagtccatgaaatctaaatcagatatcgaaatggtacataaagcgctcaaaaaacaagaagaggatccagagtataagaagaaattccgagccaatccagttcctgcatctgtctttctccccctttaccatgatttagtcaagcaaaaagaagaacggagaaggtctctgaaggagaaaagcaaagaagctcttttggcctcacaaaagccatttaaatttatagcaagggaggaacagaagcgagcagcccgggaaaagcagctgagagactttcttaagtataaaaagaaaacaaatcgatttaaagccagacccattcctcgatctacttatggttcaactaccaatgacaagttaaaagaagaagagctctatcgaaaccttaggacacagctgagagcccaggagcatttacagaactcatctcctctgccttgtaggtcagcttgcggatgcaggaaccccaggtgtcctgaacaggctgtaaagttgaagtgtaaacacaaggttaggtgcccaactcctgattttgaggaccttcctgagagataccagaaacacctctcagaacacaagtctccaaaactcttaacagtgtgtaaaccatttgatcttcatgcatctccacatgcatctattaaaagagaaaaaattttggcagacatcgaagcagatgaagaaaatttaaaagaaacacgttggccttatttgtctccaaggcgtaagtcaccagtaagatgtgcaggtgtaaaccctgtgccttgtaactgcaaccctcccgtgcccacggtatcttccagaggacgagaacaagccgtaagaaagagcgaaaaggaaaggatgagagaataccaacgagaactagaagaaagagaagaaaaattaaaaaagaggccactgctatttgaaagagttgctcagaaaaatgcaagaatggcagcagaaaagcattattctaataccctaaaagcactaggaatatctgatgagtttgtttcaaagaaaggccaaagtggaaaagtacttgagtacttcaacaatcaagagacgaaaagtgtcactgaagacaaagaaagctttaatgaagaagaaaaaatagaagaaagagagaatggggaagaaaattattttattgataccaacagccaggattcttacaaggaaaaagatgaagccaatgaggaaagtgaagaagagaaatctgttgaagaatcacactgataaaatcactagtgaattcgcggccgcctgcaggtcgacggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagaacccagctttcttgtacaaagtggtataattcctgcagcccgggggatccactagttctagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaaacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgc

SEQ ID NO: 18 - DNA, 3950 bp, synthetic construct
  source 1..3950: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggatctgaattcggtacccaagtttgtacaaaaaagcaggcttatggaggcagaggagaaggcccaggcttcccagcagggctaaggatatgcaaggagtgcattcatccggaggtgttggcagcatcccagccccaccccattctcatcgtaaatcaggctcacttccattggctgcatacggtggagtgatgtgaccatatgtcacttgagcattacacaaatcctaatgagctaaaaatatgtttgttttagctaattgacctctttggccttcataaagcaatcactagtgaattcgcggccgcctgcaggggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggcacaactttgtatacaaaagttgtatcggaggcggggccaccatggactacaaggaccacgacggtgactacaaggaccacgacatcgactacaaggacgacgacgacaagccgcggagcactatgaacttctctgatatttaccactctgatgaagagtatttcaggaaactcaaagacctgaaggctgtccatgaagaaaccatgtcaaaattagagaaaatgtaccaggacaagctaaatataaaagacatccaggcggggttcatcagggatgggatcagcgattcgtcttcgagttctgcatcggaaaagagctgctcacatcctgccttgtcagtgacctcactttcagagcctgacctagacgggtcgtcctccttgtccacaactactgatgaggggttgcccgacctagaggaaaagactcccggggaaagcagcgcaatggtccatgcccaggagctcataaacaacatgtggaacgacttttctgttgaagattatattcagtatgactcagactcccgaacagctaagaaaaaaaggaagaaagctaaatccttgacacccaagattacagtgcctgtaccctttgaaatgacggtaagagagcagaatcggagagagaaggccttgagtgctcggtcagatctggaaacaaagctgctcaagagggacgaggacgatgcagagtgtaagaagaaattccgagccaatcccgttccttcctgcgtgctccttcccctttatgaggatctagtcaagcagagtgaggagcgccggaagaaggcccgggagaggaacagagctgcgctcctggcctccctgaagccctttaagttcattgcgagggaggaacagaagcaagcagtccgcgagaagaagctgagagacctttttagggctaaaaggaaaacgaatcagtttaaagccaaacctgtgcctcgttttatttacaggccagctgcgagtgacaagccaaaggaagaagagctctatggggacagcaggatgctgccaaaggtcagagatctgttacagaattcgccttggcccagtcggtcagcttgcagacgtttcagggaccccagaagtcctgcaaagcccaggggtaaacacaggcgtaggtgcctgaggcgtgatggagacttagaaaaatggaaggagcccttctctgaatacagctttctgaaatgcccaatgctttgtgaagagtgttgtcttcacgaatccccttgtgactctgataaaagacagaaactcttggcagacatcagagcagatgaagaaattttgagagaaacacgcaggccgggtcggtctcctaggcgcaagtcaccgggaagaagctcgaacccaaagcccaggcctcatgaatgcagcccaccgatgcccaccgcgtcttcccgagggcgggagcaagccatcaggaaatcccttgaggaaaagaaaatgttggaagaagagagaaatcggatcctaactaaacagaagcaaagaatgaaagacttgcagaaactgctggcaacccgggttaaggcttatggctcccaccagagcttgtctcagatagttaaagccagggtgaaggagctcagaaggagtgaaaggccaggatgagagaatattggcaagaactggaggaacaagaggaaaaactgcagaagaggccgatgttgtttgaaagagtcacccagagaaatgcaaggatggccgcagagaggtgttactctaacactctaaaagcactaggactccttgaggagtttgtttcagagaaaggccaaagtggaaaagtgtctgaagacttcaccaggcaagagctgagaagctgcacactggagaaagaaagctcttatggagaagaaagagagaatgaggaagaaagctatttaactgatatcagcagccaggattcttgcaagggaaacaaggaagatattaaagatattaaaagtggagaagaaaattctggagaatgaatccgaatcacaaggtaatcactagtgaattcgcggccgcctgcaggtcgacggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagaacccagctttcttgtacaaagtggtgataattcctgcagcccgggggatccactagttctagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcctagagcatggctacgtagataagtagcatggcgggttaatcattaactacaaggaacccctagtgatggagttggccactccctcctctgcgcgccaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 19 - DNA, 3699 bp, synthetic construct
  source 1..3699: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggatctgaattcggtacccaagtttgtacaaaaaagcaggcttatggaggcagaggagaaggcccaggcttcccagcagggctaaggatatgcaaggagtgcattcatccggaggtgttggcagcatcccagccccaccccattctcatcgtaaatcaggctcacttccattggctgcatacggtggagtgatgtgaccatatgtcacttgagcattacacaaatcctaatgagctaaaaatatgtttgttttagctaattgacctctttggccttcataaagcaatcactagtgaattcgcggccgcctgcaggggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggcacaactttgtatacaaaagttgtatcggaggcggggccaccatggactacaaggaccacgacggtgactacaaggaccacgacatcgactacaaggacgacgacgacaagccgcgggaattcgatttcgagagcactatgaacttctctgatatttaccactctgatgaagagtatttcaggaaactcaaagacctgaaggctgtccatgaagaaaccatgtcaaaattagagaaaatgtaccaggacaagctaaatataaaagacatccaggcggggttcatcagggatgggatcagcgattcgtcttcgagttctgcatcggaaaagagctgctcacatcctgccttgtcagtgacctcactttcagagcctgacctagacgggtcgtcctccttgtccacaactactgatgaggggttgcccgacctagaggaaaagactcccggggaaagcagcgcaatggtccatgcccaggagctcataaacaacatgtggaacgacttttctgttgaagattatattcagtatgactcagactcccgaacagctaagaaaaaaaggaagaaagctaaatccttgacacccaagattacagtgcctgtaccctttgaaatgacggtaagagagcagaatcggagagagaaggccttgagtgctcggtcagatctggaaacaaagctgctcaagagggacgaggacgatgcagagtgtaagaagaaattccgagccaatcccgttccttcctgcgtgctccttcccctttatgaggatctagtcaagcagagtgaggagcgccggaagaaggcccgggagaggaacagagctgcgctcctggcctccctgaagccctttaagttcattgcgagggaggaacagaagcaagcagtccgcgagaagaagctgagagacctttttagggctaaaaggaaaacgaatcagtttaaagccaaacctgtgcctcgttttatttacaggccagctgcgagtgacaagccaaaggaagaagagctctatggggacagcaggatgctgccaaaggtcagagatctgttacagaattcgccttggcccagtcggtcagcttgcagacgtttcagggaccccagaagtcctgcaaagcccaggggtaaacacaggcgtaggtgcctgaggcgtgatggagacttagaaaaatggaaggagcccttctctgaatacagctttctgaaatgcccaatgctttgtgaagagtgttgtcttcacgaatccccttgtgactctgataaaagacagaaactcttggcagacatcagagcagatgaagaaattttgagagaaacacgcaggccgggtcggtctcctaggcgcaagtcaccgggaagaagctcgaacccaaagcccaggcctcatgaatgcagcccaccgatgcccaccgcgtcttcccgagggcgggagcaagccatcaggaaatcccttgaggaaaagaaaatgttggaagaagagagaaatcggatcctaactaaacagaagcaaagaatgaaagacttgcagaaactgctggcaacccgggttaaggcttatggctcccaccagagcttgtctcagatagttaaagccagggtgaaggagctcagaaggagtgaaaaggccaggatgagagaatattggcaagaactggaggaacaagaggaaaaactgcagaagaggccgatgttgtttgaaagagtcacccaggttgtgtttattggttagggccatcaccagctttaatcactagtgaattcgcggccgcctgcaggtcgacggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagaacccagctttcttgtacaaagtggtgataattcctgcagcccgggggatccactagttctagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcctagagcatggctacgtagataagtagcatggcgggttaatcattaactacaaggaacccctagtgatggagttggccactccctcctctgcgcgccaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 20 - DNA, 3531 bp, synthetic construct
  source 1..3531: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggatctgaattcggtacccaagtttgtacaaaaaagcaggcttatggaggcagaggagaaggcccaggcttcccagcagggctaaggatatgcaaggagtgcattcatccggaggtgttggcagcatcccagccccaccccattctcatcgtaaatcaggctcacttccattggctgcatacggtggagtgatgtgaccatatgtcacttgagcattacacaaatcctaatgagctaaaaatatgtttgttttagctaattgacctctttggccttcataaagcaatcactagtgaattcgcggccgcctgcaggggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggcacaactttgtatacaaaagttgtatcggaggcggggccaccatggactacaaggaccacgacggtgactacaaggaccacgacatcgactacaaggacgacgacgacaagccgcgggaattcgatttcgagagcactatgaacttctctgatatttaccactctgatgaagagtatttcaggaaactcaaagacctgaaggctgtccatgaagaaaccatgtcaaaattagagaaaatgtaccaggacaagctaaatataaaagacatccaggcggggttcatcagggatgggatcagcgattcgtcttcgagttctgcatcggaaaagagctgctcacatcctgccttgtcagtgacctcactttcagagcctgacctagacgggtcgtcctccttgtccacaactactgatgaggggttgcccgacctagaggaaaagactcccggggaaagcagcgcaatggtccatgcccaggagctcataaacaacatgtggaacgacttttctgttgaagattatattcagtatgactcagactcccgaacagctaagaaaaaaaggaagaaagctaaatccttgacacccaagattacagtgcctgtaccctttgaaatgacggtaagagagcagaatcggagagagaaggccttgagtgctcggtcagatctggaaacaaagctgctcaagagggacgaggacgatgcagagtgtaagaagaaattccgagccaatcccgttccttcctgcgtgctccttcccctttatgaggatctagtcaagcagagtgaggagcgccggaagaaggcccgggagaggaacagagctgcgctcctggcctccctgaagccctttaagttcattgcgagggaggaacagaagcaagcagtccgcgagaagaagctgagagacctttttagggctaaaaggaaaacgaatcagtttaaagccaaacctgtgcctcgttttatttacaggccagctgcgagtgacaagccaaaggaagaagagctctatggggacagcaggatgctgccaaaggtcagagatctgttacagaattcgccttggcccagtcggtcagcttgcagacgtttcagggaccccagaagtcctgcaaagcccaggggtaaacacaggcgtaggtgcctgaggcgtgatggagacttagaaaaatggaaggagcccttctctgaatacagctttctgaaatgcccaatgctttgtgaagagtgttgtcttcacgaatccccttgtgactctgataaaagacagaaactcttggcagacatcagagcagatgaagaaattttgagagaaacacgcaggccgggtcggtctcctaggcgcaagtcaccgggaagaagctcgaacccaaagcccaggcctcatgaatgcagcccaccgatgcccaccgcgtcttcccgagggcgggagcaagccatcagaaggagtgaaaaggccaggatgagagaatattggcaagaactggaggaacaagaggaaaaactgcagaagaggccgatgttgtttgaaagagtcacccaggttgtgtttattggttagggccatcaccagctttaatcactagtgaattcgcggccgcctgcaggtcgacggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagaacccagctttcttgtacaaagtggtgataattcctgcagcccgggggatccactagttctagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcctagagcatggctacgtagataagtagcatggcgggttaatcattaactacaaggaacccctagtgatggagttggccactccctcctctgcgcgccaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 21 - DNA, 4508 bp, synthetic construct
  source 1..4508: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggatctgaattcggtacctctctcattctttacaaattcagcgaaaaaaattttttcaaaaattacaggttgcagatgaaactatataggctgcagcacaaataattcttcataggtgctgattactaataaagcaacaggtgctggaaggattcattatctaaatgggtcaagccaatctagggaagattatcctctaatctggtggctgcatttacaacattccacaaaaagccgattaaagggagaggatgctattttaggctcttggaaaaataatgtctatggcctaggttattttgtatactaaatattttctcagtggagacaattaaagattaaattagaattggaggtcatttactagccattcatctgttttaggttaaacactgtgagatcagtttatctgaaaagtttcggctagcgttaacagtattaagctgaaaacagaatggatccagtgcaaaatcaccaaccttgtcactcttctgagaccgccacccagtaacacgccctgctttcctccgccgccgccgtctcctccatcaaccgtctccttctaacatgttattgcagttaataaaaaagcgttcttttcttcacaaaagcagttgtttcattgtgtaatgtgagaagtctgcgttcaggcggttagacgcaaaggagtggaataagtgccctgttcagcctgccaaccttcggaaccagccaacagactcggtactgactgccatgatccagtaagcacccgactcgggtctctggcagcctgagtggattttgcgtgacttttggtccctggcggctcctgtagcgtccccagttacggcgcccatagcaaccggctccctagctaggcgcccccgggttgccaggggccgcaccagctttcccgccccgggccagcgcaggcgctcaggcctcggaggcgtacgcgtcctagcgctaccggtcgccaccatggactacaaggaccacgacggtgactacaaggaccacgacatcgactacaaggacgacgacgacaagatggccacctcccaccgagtggcgaagctggtggcctccagtctccagaccccggtaaatcccatcactggagcgcgggtcgcccagtacgaacgcgaagaccccttaaaggccctggcggcagcggaggcgatcttggaggacgaagaggaggagaaagtggcgcagcccgctggggcatcggctgatttgaacaccagcttttctggggtggatgaacatgcaccgataagctatgaggactttgtgaactttcctgatattcaccactctaatgaggagtatttcaagaaagtagaagagttgaaggctgcccacatagaaactatggcaaaattagagaaaatgtaccaggataaattacatttaaaggaagttcagccagtggtcatcagagaagactctcttagtgactcttccagatctgtatcagaaaagaactcctatcaccctgtctcattaatgacatcattttcagagcctgatttaggccagtcttcctccttgtatgtgtcctcctctgaagaggagttacccaacctagaaaaagagtatcctaggaaaaacagaatgatgacctatgctaaggagctcatcaacaatatgtggacagacttttgtgttgaggattatattcgctgtaaagatactggcttccatgcagctgaaaaaagaaggaagaaacgaaaagaatgggtgcccacaattacagtaccggagccttttcaaatgatgataagagaacagaagaaaaaagaagagtccatgaaatctaaatcagatatcgaaatggtacataaagcgctcaaaaaacaagaagaggatccagagtataagaagaaattccgagccaatccagttcctgcatctgtctttctccccctttaccatgatttagtcaagcaaaaagaagaacggagaaggtctctgaaggagaaaagcaaagaagctcttttggcctcacaaaagccatttaaatttatagcaagggaggaacagaagcgagcagcccgggaaaagcagctgagagactttcttaagtataaaaagaaaacaaatcgatttaaagccagacccattcctcgatctacttatggttcaactaccaatgacaagttaaaagaagaagagctctatcgaaaccttaggacacagctgagagcccaggagcatttacagaactcatctcctctgccttgtaggtcagcttgtggatgcaggaaccccaggtgtcctgaacaggctgtaaagttgaagtgtaaacacaaggttaggtgcccaactcctgattttgaggaccttcctgagagataccagaaacacctctcagaacacaagtctccaaaactcttaacagtgtgtaaaccatttgatcttcatgcatctccacatgcatctattaaaagagaaaaaattttggcagacatcgaagcagatgaagaaaatttaaaagaaacacgttggccttatttgtctccaaggcgtaagtcaccagtaagatgtgcaggtgtaaaccctgtgccttgtaactgcaaccctcccgtgcccacggtatcttccagaggacgagaacaagccgtaaggagatcacttgaggaaaagaaaatgttggaagaagagagaaatcggatcctaactaaacagaagcaaagaatgaaagaattgcagaaactcctgacaacccgggctaaggcttatgactcacatcaaagtttagctcaaatatctaaatccagagtaaaatgtctcagaaagagcgaaaaggaaaggatgagagaataccaacgagaactagaagaaagagaagaaaaattaaaaaagaggccactgctatttgaaagagttgctcagaaaaatgcaagaatggcagcagaaaagcattattctaataccctaaaagcactaggaatatctgatgagtttgtttcaaagaaaggccaaagtggaaaagtacttgagtacttcaacaatcaagagacgaaaagtgtcactgaagacaaagaaagctttaatgaagaagaaaaaatagaagaaagagagaatggggaagaaaattattttattgataccaacagccaggattcttacaaggaaaaagatgaagccaatgaggaaagtgaagaagagaaatctgttgaagaatcacactgagcggccgcctgcaggtcgacggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagaacccagctttcttgtacaaagtggtgataattcctgcagcccgggggatccactagttctagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcctagagcatggctacgtagataagtagcatggcgggttaatcattaactacaaggaacccctagtgatggagttggccactccctcctctgcgcgccaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 22 - DNA, 4327 bp, synthetic construct
  source 1..4327: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggatctgaattcggtacctctctcattctttacaaattcagcgaaaaaaattttttcaaaaattacaggttgcagatgaaactatataggctgcagcacaaataattcttcataggtgctgattactaataaagcaacaggtgctggaaggattcattatctaaatgggtcaagccaatctagggaagattatcctctaatctggtggctgcatttacaacattccacaaaaagccgattaaagggagaggatgctattttaggctcttggaaaaataatgtctatggcctaggttattttgtatactaaatattttctcagtggagacaattaaagattaaattagaattggaggtcatttactagccattcatctgttttaggttaaacactgtgagatcagtttatctgaaaagtttcggctagcgttaacagtattaagctgaaaacagaatggatccagtgcaaaatcaccaaccttgtcactcttctgagaccgccacccagtaacacgccctgctttcctccgccgccgccgtctcctccatcaaccgtctccttctaacatgttattgcagttaataaaaaagcgttcttttcttcacaaaagcagttgtttcattgtgtaatgtgagaagtctgcgttcaggcggttagacgcaaaggagtggaataagtgccctgttcagcctgccaaccttcggaaccagccaacagactcggtactgactgccatgatccagtaagcacccgactcgggtctctggcagcctgagtggattttgcgtgacttttggtccctggcggctcctgtagcgtccccagttacggcgcccatagcaaccggctccctagctaggcgcccccgggttgccaggggccgcaccagctttcccgccccgggccagcgcaggcgctcaggcctcggaggcgtacgcgtcctagcgctaccggtcgccaccatggactacaaggaccacgacggtgactacaaggaccacgacatcgactacaaggacgacgacgacaagatggccacctcccaccgagtggcgaagctggtggcctccagtctccagaccccggtaaatcccatcactggagcgcgggtcgcccagtacgaacgcgaagaccccttaaaggccctggcggcagcggaggcgatcttggaggacgaagaggaggagaaagtggcgcagcccgctggggcatcggctgatttgaacaccagcttttctggggtggatgaacatgcaccgataagctatgaggactttgtgaactttcctgatattcaccactctaatgaggagtatttcaagaaagtagaagagttgaaggctgcccacatagaaactatggcaaaattagagaaaatgtaccaggataaattacatttaaaggaagttcagccagtggtcatcagagaagactctcttagtgactcttccagatctgtatcagaaaagaactcctatcaccctgtctcattaatgacatcattttcagagcctgatttaggccagtcttcctccttgtatgtgtcctcctctgaagaggagttacccaacctagaaaaagagtatcctaggaaaaacagaatgatgacctatgctaaggagctcatcaacaatatgtggacagacttttgtgttgaggattatattcgctgtaaagatactggcttccatgcagctgaaaaaagaaggaagaaacgaaaagaatgggtgcccacaattacagtaccggagccttttcaaatgatgataagagaacagaagaaaaaagaagagtccatgaaatctaaatcagatatcgaaatggtacataaagcgctcaaaaaacaagaagaggatccagagtataagaagaaattccgagccaatccagttcctgcatctgtctttctccccctttaccatgatttagtcaagcaaaaagaagaacggagaaggtctctgaaggagaaaagcaaagaagctcttttggcctcacaaaagccatttaaatttatagcaagggaggaacagaagcgagcagcccgggaaaagcagctgagagactttcttaagtataaaaagaaaacaaatcgatttaaagccagacccattcctcgatctacttatggttcaactaccaatgacaagttaaaagaagaagagctctatcgaaaccttaggacacagctgagagcccaggagcatttacagaactcatctcctctgccttgtaggtcagcttgcggatgcaggaaccccaggtgtcctgaacaggctgtaaagttgaagtgtaaacacaaggttaggtgcccaactcctgattttgaggaccttcctgagagataccagaaacacctctcagaacacaagtctccaaaactcttaacagtgtgtaaaccatttgatcttcatgcatctccacatgcatctattaaaagagaaaaaattttggcagacatcgaagcagatgaagaaaatttaaaagaaacacgttggccttatttgtctccaaggcgtaagtcaccagtaagatgtgcaggtgtaaaccctgtgccttgtaactgcaaccctcccgtgcccacggtatcttccagaggacgagaacaagccgtaagaaagagcgaaaaggaaaggatgagagaataccaacgagaactagaagaaagagaagaaaaattaaaaaagaggccactgctatttgaaagagttgctcagaaaaatgcaagaatggcagcagaaaagcattattctaataccctaaaagcactaggaatatctgatgagtttgtttcaaagaaaggccaaagtggaaaagtacttgagtacttcaacaatcaagagacgaaaagtgtcactgaagacaaagaaagctttaatgaagaagaaaaaatagaagaaagagagaatggggaagaaaattattttattgataccaacagccaggattcttacaaggaaaaagatgaagccaatgaggaaagtgaagaagagaaatctgttgagcggccgcctgcaggtcgacggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagaacccagctttcttgtacaaagtggtgataattcctgcagcccgggggatccactagttctagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcctagagcatggctacgtagataagtagcatggcgggttaatcattaactacaaggaacccctagtgatggagttggccactccctcctctgcgcgccaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 23 - DNA, 4220 bp, synthetic construct
  source 1..4220: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggatctgaattcggtacctctctcattctttacaaattcagcgaaaaaaattttttcaaaaattacaggttgcagatgaaactatataggctgcagcacaaataattcttcataggtgctgattactaataaagcaacaggtgctggaaggattcattatctaaatgggtcaagccaatctagggaagattatcctctaatctggtggctgcatttacaacattccacaaaaagccgattaaagggagaggatgctattttaggctcttggaaaaataatgtctatggcctaggttattttgtatactaaatattttctcagtggagacaattaaagattaaattagaattggaggtcatttactagccattcatctgttttaggttaaacactgtgagatcagtttatctgaaaagtttcggctagcgttaacagtattaagctgaaaacagaatggatccagtgcaaaatcaccaaccttgtcactcttctgagaccgccacccagtaacacgccctgctttcctccgccgccgccgtctcctccatcaaccgtctccttctaacatgttattgcagttaataaaaaagcgttcttttcttcacaaaagcagttgtttcattgtgtaatgtgagaagtctgcgttcaggcggttagacgcaaaggagtggaataagtgccctgttcagcctgccaaccttcggaaccagccaacagactcggtactgactgccatgatccagtaagcacccgactcgggtctctggcagcctgagtggattttgcgtgacttttggtccctggcggctcctgtagcgtccccagttacggcgcccatagcaaccggctccctagctaggcgcccccgggttgccaggggccgcaccagctttcccgccccgggccagcgcaggcgctcaggcctcggaggcgtacgcgtcctagcgctaccggtcgccaccatggactacaaggaccacgacggtgactacaaggaccacgacatcgactacaaggacgacgacgacaagatgaacttctctgatatttaccactctgatgaagagtatttcaggaaactcaaagacctgaaggctgtccatgaagaaaccatgtcaaaattagagaaaatgtaccaggacaagctaaatataaaagacatccaggcggggttcatcagggatgggatcagcgattcgtcttcgagttctgcatcggaaaagagctgctcacatcctgccttgtcagtgacctcactttcagagcctgacctagacgggtcgtcctccttgtccacaactactgatgaggggttgcccgacctagaggaaaagactcccggggaaagcagcgcaatggtccatgcccaggagctcataaacaacatgtggaacgacttttctgttgaagattatattcagtatgactcagactcccgaacagctaagaaaaaaaggaagaaagctaaatccttgacacccaagattacagtgcctgtaccctttgaaatgacggtaagagagcagaatcggagagagaaggccttgagtgctcggtcagatctggaaacaaagctgctcaagagggacgaggacgatgcagagtgtaagaagaaattccgagccaatcccgttccttcctgcgtgctccttcccctttatgaggatctagtcaagcagagtgaggagcgccggaagaaggcccgggagaggaacagagctgcgctcctggcctccctgaagccctttaagttcattgcgagggaggaacagaagcaagcagtccgcgagaagaagctgagagacctttttagggctaaaaggaaaacgaatcagtttaaagccaaacctgtgcctcgttttatttacaggccagctgcgagtgacaagccaaaggaagaagagctctatggggacagcaggatgctgccaaaggtcagagatctgttacagaattcgccttggcccagtcggtcagcttgcagacgtttcagggaccccagaagtcctgcaaagcccaggggtaaacacaggcgtaggtgcctgaggcgtgatggagacttagaaaaatggaaggagcccttctctgaatacagctttctgaaatgcccaatgctttgtgaagagtgttgtcttcacgaatccccttgtgactctgataaaagacagaaactcttggcagacatcagagcagatgaagaaattttgagagaaacacgcaggccgggtcggtctcctaggcgcaagtcaccgggaagaagctcgaacccaaagcccaggcctcatgaatgcagcccaccgatgcccaccgcgtcttcccgagggcgggagcaagccatcaggaaatcccttgaggaaaagaaaatgttggaagaagagagaaatcggatcctaactaaacagaagcaaagaatgaaagacttgcagaaactgctggcaacccgggttaaggcttatggctcccaccagagcttgtctcagatagttaaagccagggtgaaggagctcagaaggagtgaaaaggccaggatgagagaatattggcaagaactggaggaacaagaggaaaaactgcagaagaggccgatgttgtttgaaagagtcacccagagaaatgcaaggatggccgcagagaggtgttactctaacactctaaaagcactaggactccttgaggagtttgtttcagagaaaggccaaagtggaaaagtgtctgaagacttcaccaggcaagagctgagaagctgcacactggagaaagaaagctcttatggagaagaaagagagaatgaggaagaaagctatttaactgatatcagcagccaggattcttgcaagggaaacaaggaagatattaaagatattaaaagtggagaagaaaattctggagaatgagcggccgcctgcaggtcgacggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagaacccagctttcttgtacaaagtggtgataattcctgcagcccgggggatccactagttctagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcctagagcatggctacgtagataagtagcatggcgggttaatcattaactacaaggaacccctagtgatggagttggccactccctcctctgcgcgccaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 24 - DNA, 4051 bp, synthetic construct
  source 1..4051: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggatctgaattcggtacctctctcattctttacaaattcagcgaaaaaaattttttcaaaaattacaggttgcagatgaaactatataggctgcagcacaaataattcttcataggtgctgattactaataaagcaacaggtgctggaaggattcattatctaaatgggtcaagccaatctagggaagattatcctctaatctggtggctgcatttacaacattccacaaaaagccgattaaagggagaggatgctattttaggctcttggaaaaataatgtctatggcctaggttattttgtatactaaatattttctcagtggagacaattaaagattaaattagaattggaggtcatttactagccattcatctgttttaggttaaacactgtgagatcagtttatctgaaaagtttcggctagcgttaacagtattaagctgaaaacagaatggatccagtgcaaaatcaccaaccttgtcactcttctgagaccgccacccagtaacacgccctgctttcctccgccgccgccgtctcctccatcaaccgtctccttctaacatgttattgcagttaataaaaaagcgttcttttcttcacaaaagcagttgtttcattgtgtaatgtgagaagtctgcgttcaggcggttagacgcaaaggagtggaataagtgccctgttcagcctgccaaccttcggaaccagccaacagactcggtactgactgccatgatccagtaagcacccgactcgggtctctggcagcctgagtggattttgcgtgacttttggtccctggcggctcctgtagcgtccccagttacggcgcccatagcaaccggctccctagctaggcgcccccgggttgccaggggccgcaccagctttcccgccccgggccagcgcaggcgctcaggcctcggaggcgtacgcgtcctagcgctaccggtcgccaccatggactacaaggaccacgacggtgactacaaggaccacgacatcgactacaaggacgacgacgacaagatgaacttctctgatatttaccactctgatgaagagtatttcaggaaactcaaagacctgaaggctgtccatgaagaaaccatgtcaaaattagagaaaatgtaccaggacaagctaaatataaaagacatccaggcggggttcatcagggatgggatcagcgattctcttcgagttctgcatcggaaaagagctgctcacatcctgccttgtcagtgacctcactttcagagcctgacctagacgggtcgtcctccttgtccacaactactgatgaggggttgcccgacctagaggaaaagactcccggggaaagcagcgcaatggtccatgcccaggagctcataaacaacatgtggaacgacttttctgttgaagattatattcagtatgactcagactcccgaacagctaagaaaaaaaggaagaaagctaaatccttgacacccaagattacagtgcctgtaccctttgaaatgacggtaagagagcagaatcggagagagaaggccttgagtgctcggtcagatctggaaacaaagctgctcaagagggacgaggacgatgcagagtgtaagaagaaattccgagccaatcccgttccttcctgcgtgctccttcccctttatgaggatctagtcaagcagagtgaggagcgccggaagaaggcccgggagaggaacagagctgcgctcctggcctccctgaagccctttaagttcattgcgagggaggaacagaagcaagcagtccgcgagaagaagctgagagacctttttagggctaaaaggaaaacgaatcagtttaaagccaaacctgtgcctcgttttatttacaggccagctgcgagtgacaagccaaaggaagaagagctctatggggacagcaggatgctgccaaaggtcagagatctgttacagaattcgccttggcccagtcggtcagcttgcagacgtttcagggaccccagaagtcctgcaaagcccaggggtaaacacaggcgtaggtgcctgaggcgtgatggagacttagaaaaatggaaggagcccttctctgaatacagctttctgaaatgcccaatgctttgtgaagagtgttgtcttcacgaatccccttgtgactctgataaaagacagaaactcttggcagacatcagagcagatgaagaaattttgagagaaacacgcaggccgggtcggtctcctaggcgcaagtcaccgggaagaagctcgaacccaaagcccaggcctcatgaatgcagcccaccgatgcccaccgcgtcttcccgagggcgggagcaagccatcagaaggagtgaaaaggccaggatgagagaatattggcaagaactggaggaacaagaggaaaaactgcagaagaggccgatgttgtttgaaagagtcacccagagaaatgcaaggatggccgcagagaggtgttactctaacactctaaaagcactaggactccttgaggagtttgtttcagagaaaggccaaagtggaaaagtgtctgaagacttcaccaggcaagagctgagaagctgcacactggagaaagaaagctcttatggagaagaaagagagaatgaggaagaaagctatttaactgatatcagcagccaggattcttgcaagggaaacaaggaagatattaaagatattaaaagtggagaagaaaattctggagaatgagcggccgcctgcaggtcgacggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagaacccagctttcttgtacaaagtggtgataattcctgcagcccgggggatccactagttctagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcctagagcatggctacgtagataagtagcatggcgggttaatcattaactacaaggaacccctagtgatggagttggccactccctcctctgcgcgccaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 25 - DNA, 4659 bp, synthetic construct
  source 1..4659: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggatctgaattcggtacgggctcagaccttagagacgggttctcgacgatgtgcagcgcataacgaggaaaatactgaccggagggggccggtcgtagggctggggagcaggaggagggcaaaggatagagagggggtagatagggagtagatagggggcggcagcctcggcctcagtaagcaggggctcagggagcagatgtctgagactgcaccacgtccgctattttgaggtttggcttctcttactgccacgtggtgcagagggaaggcgccatttgatgcgtccaaatcccgagtgggaagagaagatacggatgagggagatggatgtgtctgtggtgactcttgggcacttgtgccgttcaatcctgtcactctcaagctgaagaacccaaagcaaacgccaggtttacaatacctgcactatcgctcattagtgggagggtcaggcgccacccaactctgggcttttattggtcatgtcaagaaaaaagcgagagggacaactgggagcctccttctgattggtggattcctgctagccctcaagtcaatcagagtagaatgtaccgccctccttcccactctggcccccgcgggagacatgttaggtagcgggacgccaaccttgtcactcttctgagaccgccacccagtaacacgccctgctttcctccgccgccgccgtctcctccatcaaccgtctccttctaacatgttattgcagttaataaaaaagcgttcttttcttcacaaaagcagttgtttcattgtgtaatgtgagaagtctgcgttcaggcggttagacgcaaaggagtggaataagtgccctgttcagcctgccaaccttcggaaccagccaacagactcggtactgactgccatgatccagtaagcacccgactcgggtctctggcagcctgagtggattttgcgtgacttttggtccctggcggctcctgtagcgtccccagttacggcgcccatagcaaccggctccctagctaggcgcccccgggttgccaggggccgcaccagctttcccgccccgggccagcgcaggcgctcaggcctcggaggcgtacgcgtcctagcgctaccggtcgccaccatggactacaaggaccacgacggtgactacaaggaccacgacatcgactacaaggacgacgacgacaagatggccacctcccaccgagtggcgaagctggtggcctccagtctccagaccccggtaaatcccatcactggagcgcgggtcgcccagtacgaacgcgaagaccccttaaaggccctggcggcagcggaggcgatcttggaggacgaagaggaggagaaagtggcgcagcccgctggggcatcggctgatttgaacaccagcttttctggggtggatgaacatgcaccgataagctatgaggactttgtgaactttcctgatattcaccactctaatgaggagtatttcaagaaagtagaagagttgaaggctgcccacatagaaactatggcaaaattagagaaaatgtaccaggataaattacatttaaaggaagttcagccagtggtcatcagagaagactctcttagtgactcttccagatctgtatcagaaaagaactcctatcaccctgtctcattaatgacatcattttcagagcctgatttaggccagtcttcctccttgtatgtgtcctcctctgaagaggagttacccaacctagaaaaagagtatcctaggaaaaacagaatgatgacctatgctaaggagctcatcaacaatatgtggacagacttttgtgttgaggattatattcgctgtaaagatactggcttccatgcagctgaaaaaagaaggaagaaacgaaaagaatgggtgcccacaattacagtaccggagccttttcaaatgatgataagagaacagaagaaaaaagaagagtccatgaaatctaaatcagatatcgaaatggtacataaagcgctcaaaaaacaagaagaggatccagagtataagaagaaattccgagccaatccagttcctgcatctgtctttctccccctttaccatgatttagtcaagcaaaaagaagaacggagaaggtctctgaaggagaaaagcaaagaagctcttttggcctcacaaaagccatttaaatttatagcaagggaggaacagaagcgagcagcccgggaaaagcagctgagagactttcttaagtataaaaagaaaacaaatcgatttaaagccagacccattcctcgatctacttatggttcaactaccaatgacaagttaaaagaagaagagctctatcgaaaccttaggacacagctgagagcccaggagcatttacagaactcatctcctctgccttgtaggtcagcttgtggatgcaggaaccccaggtgtcctgaacaggctgtaaagttgaagtgtaaacacaaggttaggtgcccaactcctgattttgaggaccttcctgagagataccagaaacacctctcagaacacaagtctccaaaactcttaacagtgtgtaaaccatttgatcttcatgcatctccacatgcatctattaaaagagaaaaaattttggcagacatcgaagcagatgaagaaaatttaaaagaaacacgttggccttatttgtctccaaggcgtaagtcaccagtaagatgtgcaggtgtaaaccctgtgccttgtaactgcaaccctcccgtgcccacggtatcttccagaggacgagaacaagccgtaaggagatcacttgaggaaaagaaaatgttggaagaagagagaaatcggatcctaactaaacagaagcaaagaatgaaagaattgcagaaactcctgacaacccgggctaaggcttatgactcacatcaaagtttagctcaaatatctaaatccagagtaaaatgtctcagaaagagcgaaaaggaaaggatgagagaataccaacgagaactagaagaaagagaagaaaaattaaaaaagaggccactgctatttgaaagagttgctcagaaaaatgcaagaatggcagcagaaaagcattattctaataccctaaaagcactaggaatatctgatgagtttgtttcaaagaaaggccaaagtggaaaagtacttgagtacttcaacaatcaagagacgaaaagtgtcactgaagacaaagaaagctttaatgaagaagaaaaaatagaagaaagagagaatggggaagaaaattattttattgataccaacagccaggattcttacaaggaaaaagatgaagccaatgaggaaagtgaagaagagaaatctgttgaagaatcacactgagcggccgcctgcaggtcgacggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagaacccagctttcttgtacaaagtggtgataattcctgcagcccgggggatccactagttctagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcctagagcatggctacgtagataagtagcatggcgggttaatcattaactacaaggaacccctagtgatggagttggccactccctcctctgcgcgccaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 26 - DNA, 4478 bp, synthetic construct
  source 1..4478: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggatctgaattcggtacgggctcagaccttagagacgggttctcgacgatgtgcagcgcataacgaggaaaatactgaccggagggggccggtcgtagggctggggagcaggaggagggcaaaggatagagagggggtagatagggagtagatagggggcggcagcctcggcctcagtaagcaggggctcagggagcagatgtctgagactgcaccacgtccgctattttgaggtttggcttctcttactgccacgtggtgcagagggaaggcgccatttgatgcgtccaaatcccgagtgggaagagaagatacggatgagggagatggatgtgtctgtggtgactcttgggcacttgtgccgttcaatcctgtcactctcaagctgaagaacccaaagcaaacgccaggtttacaatacctgcactatcgctcattagtgggagggtcaggcgccacccaactctgggcttttattggtcatgtcaagaaaaaagcgagagggacaactgggagcctccttctgattggtggattcctgctagccctcaagtcaatcagagtagaatgtaccgccctccttcccactctggcccccgcgggagacatgttaggtagcgggacgccaaccttgtcactcttctgagaccgccacccagtaacacgccctgctttcctccgccgccgccgtctcctccatcaaccgtctccttctaacatgttattgcagttaataaaaaagcgttcttttcttcacaaaagcagttgtttcattgtgtaatgtgagaagtctgcgttcaggcggttagacgcaaaggagtggaataagtgccctgttcagcctgccaaccttcggaaccagccaacagactcggtactgactgccatgatccagtaagcacccgactcgggtctctggcagcctgagtggattttgcgtgacttttggtccctggcggctcctgtagcgtccccagttacggcgcccatagcaaccggctccctagctaggcgcccccgggttgccaggggccgcaccagctttcccgccccgggccagcgcaggcgctcaggcctcggaggcgtacgcgtcctagcgctaccggtcgccaccatggactacaaggaccacgacggtgactacaaggaccacgacatcgactacaaggacgacgacgacaagatggccacctcccaccgagtggcgaagctggtggcctccagtctccagaccccggtaaatcccatcactggagcgcgggtcgcccagtacgaacgcgaagaccccttaaaggccctggcggcagcggaggcgatcttggaggacgaagaggaggagaaagtggcgcagcccgctggggcatcggctgatttgaacaccagcttttctggggtggatgaacatgcaccgataagctatgaggactttgtgaactttcctgatattcaccactctaatgaggagtatttcaagaaagtagaagagttgaaggctgcccacatagaaactatggcaaaattagagaaaatgtaccaggataaattacatttaaaggaagttcagccagtggtcatcagagaagactctcttagtgactcttccagatctgtatcagaaaagaactcctatcaccctgtctcattaatgacatcattttcagagcctgatttaggccagtcttcctccttgtatgtgtcctcctctgaagaggagttacccaacctagaaaaagagtatcctaggaaaaacagaatgatgacctatgctaaggagctcatcaacaatatgtggacagacttttgtgttgaggattatattcgctgtaaagatactggcttccatgcagctgaaaaaagaaggaagaaacgaaaagaatgggtgcccacaattacagtaccggagccttttcaaatgatgataagagaacagaagaaaaaagaagagtccatgaaatctaaatcagatatcgaaatggtacataaagcgctcaaaaaacaagaagaggatccagagtataagaagaaattccgagccaatccagttcctgcatctgtctttctccccctttaccatgatttagtcaagcaaaaagaagaacggagaaggtctctgaaggagaaaagcaaagaagctcttttggcctcacaaaagccatttaaatttatagcaagggaggaacagaagcgagcagcccgggaaaagcagctgagagactttcttaagtataaaaagaaaacaaatcgatttaaagccagacccattcctcgatctacttatggttcaactaccaatgacaagttaaaagaagaagagctctatcgaaaccttaggacacagctgagagcccaggagcatttacagaactcatctcctctgccttgtaggtcagcttgcggatgcaggaaccccaggtgtcctgaacaggctgtaaagttgaagtgtaaacacaaggttaggtgcccaactcctgattttgaggaccttcctgagagataccagaaacacctctcagaacacaagtctccaaaactcttaacagtgtgtaaaccatttgatcttcatgcatctccacatgcatctattaaaagagaaaaaattttggcagacatcgaagcagatgaagaaaatttaaaagaaacacgttggccttatttgtctccaaggcgtaagtcaccagtaagatgtgcaggtgtaaaccctgtgccttgtaactgcaaccctcccgtgcccacggtatcttccagaggacgagaacaagccgtaagaaagagcgaaaaggaaaggatgagagaataccaacgagaactagaagaaagagaagaaaaattaaaaaagaggccactgctatttgaaagagttgctcagaaaaatgcaagaatggcagcagaaaagcattattctaataccctaaaagcactaggaatatctgatgagtttgtttcaaagaaaggccaaagtggaaaagtacttgagtacttcaacaatcaagagacgaaaagtgtcactgaagacaaagaaagctttaatgaagaagaaaaaatagaagaaagagagaatggggaagaaaattattttattgataccaacagccaggattcttacaaggaaaaagatgaagccaatgaggaaagtgaagaagagaaatctgttgagcggccgcctgcaggtcgacggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagaacccagctttcttgtacaaagtggtgataattcctgcagcccgggggatccactagttctagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcctagagcatggctacgtagataagtagcatggcgggttaatcattaactacaaggaacccctagtgatggagttggccactccctcctctgcgcgccaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 27 - DNA, 4371 bp, synthetic construct
  source 1..4371: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggatctgaattcggtacgggctcagaccttagagacgggttctcgacgatgtgcagcgcataacgaggaaaatactgaccggagggggccggtcgtagggctggggagcaggaggagggcaaaggatagagagggggtagatagggagtagatagggggcggcagcctcggcctcagtaagcaggggctcagggagcagatgtctgagactgcaccacgtccgctattttgaggtttggcttctcttactgccacgtggtgcagagggaaggcgccatttgatgcgtccaaatcccgagtgggaagagaagatacggatgagggagatggatgtgtctgtggtgactcttgggcacttgtgccgttcaatcctgtcactctcaagctgaagaacccaaagcaaacgccaggtttacaatacctgcactatcgctcattagtgggagggtcaggcgccacccaactctgggcttttattggtcatgtcaagaaaaaagcgagagggacaactgggagcctccttctgattggtggattcctgctagccctcaagtcaatcagagtagaatgtaccgccctccttcccactctggcccccgcgggagacatgttaggtagcgggacgccaaccttgtcactcttctgagaccgccacccagtaacacgccctgctttcctccgccgccgccgtctcctccatcaaccgtctccttctaacatgttattgcagttaataaaaaagcgttcttttcttcacaaaagcagttgtttcattgtgtaatgtgagaagtctgcgttcaggcggttagacgcaaaggagtggaataagtgccctgttcagcctgccaaccttcggaaccagccaacagactcggtactgactgccatgatccagtaagcacccgactcgggtctctggcagcctgagtggattttgcgtgacttttggtccctggcggctcctgtagcgtccccagttacggcgcccatagcaaccggctccctagctaggcgcccccgggttgccaggggccgcaccagctttcccgccccgggccagcgcaggcgctcaggcctcggaggcgtacgcgtcctagcgctaccggtcgccaccatggactacaaggaccacgacggtgactacaaggaccacgacatcgactacaaggacgacgacgacaagatgaacttctctgatatttaccactctgatgaagagtatttcaggaaactcaaagacctgaaggctgtccatgaagaaaccatgtcaaaattagagaaaatgtaccaggacaagctaaatataaaagacatccaggcggggttcatcagggatgggatcagcgattcgtcttcgagttctgcatcggaaaagagctgctcacatcctgccttgtcagtgacctcactttcagagcctgacctagacgggtcgtcctccttgtccacaactactgatgaggggttgcccgacctagaggaaaagactcccggggaaagcagcgcaatggtccatgcccaggagctcataaacaacatgtggaacgacttttctgttgaagattatattcagtatgactcagactcccgaacagctaagaaaaaaaggaagaaagctaaatccttgacacccaagattacagtgcctgtaccctttgaaatgacggtaagagagcagaatcggagagagaaggccttgagtgctcggtcagatctggaaacaaagctgctcaagagggacgaggacgatgcagagtgtaagaagaaattccgagccaatcccgttccttcctgcgtgctccttcccctttatgaggatctagtcaagcagagtgaggagcgccggaagaaggcccgggagaggaacagagctgcgctcctggcctccctgaagccctttaagttcattgcgagggaggaacagaagcaagcagtccgcgagaagaagctgagagacctttttagggctaaaaggaaaacgaatcagtttaaagccaaacctgtgcctcgttttatttacaggccagctgcgagtgacaagccaaaggaagaagagctctatggggacagcaggatgctgccaaaggtcagagatctgttacagaattcgccttggcccagtcggtcagcttgcagacgtttcagggaccccagaagtcctgcaaagcccaggggtaaacacaggcgtaggtgcctgaggcgtgatggagacttagaaaaatggaaggagcccttctctgaatacagctttctgaaatgcccaatgctttgtgaagagtgttgtcttcacgaatccccttgtgactctgataaaagacagaaactcttggcagacatcagagcagatgaagaaattttgagagaaacacgcaggccgggtcggtctcctaggcgcaagtcaccgggaagaagctcgaacccaaagcccaggcctcatgaatgcagcccaccgatgcccaccgcgtcttcccgagggcgggagcaagccatcaggaaatcccttgaggaaaagaaaatgttggaagaagagagaaatcggatcctaactaaacagaagcaaagaatgaaagacttgcagaaactgctggcaacccgggttaaggcttatggctcccaccagagcttgtctcagatagttaaagccagggtgaaggagctcagaaggagtgaaaaggccaggatgagagaatattggcaagaactggaggaacaagaggaaaaactgcagaagaggccgatgttgtttgaaagagtcacccagagaaatgcaaggatggccgcagagaggtgttactctaacactctaaaagcactaggactccttgaggagtttgtttcagagaaaggccaaagtggaaaagtgtctgaagacttcaccaggcaagagctgagaagctgcacactggagaaagaaagctcttatggagaagaaagagagaatgaggaagaaagctatttaactgatatcagcagccaggattcttgcaagggaaacaaggaagatattaaagatattaaaagtggagaagaaaattctggagaatgagcggccgcctgcaggtcgacggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagaacccagctttcttgtacaaagtggtgataattcctgcagcccgggggatccactagttctagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcctagagcatggctacgtagataagtagcatggcgggttaatcattaactacaaggaacccctagtgatggagttggccactccctcctctgcgcgccaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 28 - DNA, 4660 bp, synthetic construct
  source 1..4660: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggatctgaattcggtacctctctcattctttacaaattcagcgaaaaaaattttttcaaaaattacaggttgcagatgaaactatataggctgcagcacaaataattcttcataggtgctgattactaataaagcaacaggtgctggaaggattcattatctaaatgggtcaagccaatctagggaagattatcctctaatctggtggctgcatttacaacattccacaaaaagccgattaaagggagaggatgctattttaggctcttggaaaaataatgtctatggcctaggttattttgtatactaaatattttctcagtggagacaattaaagattaaattagaattggaggtcatttactagccattcatctgttttaggttaaacactgtgagatcagtttatctgaaaagtttcggctagcgttaacagtattaagctgaaaacagaatggatccagtgcaaaatcagggctcagaccttagagacgggttctcgacgatgtgcagcgcataacgaggaaaatactgaccggagggggccggtcgtagggctggggagcaggaggagggcaaaggatagagagggggtagatagggagtagatagggggcggcagcctcggcctcagtaagcaggggctcagggagcagatgtctgagactgcaccacgtccgctattttgaggtttggcttctcttactgccacgtggtgcagagggaaggcgccatttgatgcgtccaaatcccgagtgggaagagaagatacggatgagggagatggatgtgtctgtggtgactcttgggcacttgtgccgttcaatcctgtcactctcaagctgaagaacccaaagcaaacgccaggtttacaatacctgcactatcgctcattagtgggagggtcaggcgccacccaactctgggcttttattggtcatgtcaagaaaaaagcgagagggacaactgggagcctccttctgattggtggattcctgctagccctcaagtcaatcagagtagaatgtaccgccctccttcccactctggcccccgcgggagacatgttaggtagcgggacgccaaccttgtcactcttctgagaccgccacccagtaacacgccctgctttcctccgccgccgccgtctcctccatcaaccgtctccttctaacatgttattgcagttaataaaaaagcgttcttttcttcacaaaagcagttgtttcattgtgtaatgtgagaagtctgcgttcaggcggttagacgcaaaggagtggaataagtgccctgttcagcctgccaaccttcggaaccagccaacagactcggtactgactgccatgatccagtaagcacccgactcgggtctctggcagcctgagtggattttgcgtgacttttggtccctggcggctcctgtagcgtccccagttacggcgcccatagcaaccggctccctagctaggcgcccccgggttgccaggggccgcaccagctttcccgccccgggccagcgcaggcgctcaggcctcggaggcgtacgcgtcctagcgctaccggtcgccaccatggactacaaggaccacgacggtgactacaaggaccacgacatcgactacaaggacgacgacgacaagatgaacttctctgatatttaccactctgatgaagagtatttcaggaaactcaaagacctgaaggctgtccatgaagaaaccatgtcaaaattagagaaaatgtaccaggacaagctaaatataaaagacatccaggcggggttcatcagggatgggatcagcgattcgtcttcgagttctgcatcggaaaagagctgctcacatcctgccttgtcagtgacctcactttcagagcctgacctagacgggtcgtcctccttgtccacaactactgatgaggggttgcccgacctagaggaaaagactcccggggaaagcagcgcaatggtccatgcccaggagctcataaacaacatgtggaacgacttttctgttgaagattatattcagtatgactcagactcccgaacagctaagaaaaaaaggaagaaagctaaatccttgacacccaagattacagtgcctgtaccctttgaaatgacggtaagagagcagaatcggagagagaaggccttgagtgctcggtcagatctggaaacaaagctgctcaagagggacgaggacgatgcagagtgtaagaagaaattccgagccaatcccgttccttcctgcgtgctccttcccctttatgaggatctagtcaagcagagtgaggagcgccggaagaaggcccgggagaggaacagagctgcgctcctggcctccctgaagccctttaagttcattgcgagggaggaacagaagcaagcagtccgcgagaagaagctgagagacctttttagggctaaaaggaaaacgaatcagtttaaagccaaacctgtgcctcgttttatttacaggccagctgcgagtgacaagccaaaggaagaagagctctatggggacagcaggatgctgccaaaggtcagagatctgttacagaattcgccttggcccagtcggtcagcttgcagacgtttcagggaccccagaagtcctgcaaagcccaggggtaaacacaggcgtaggtgcctgaggcgtgatggagacttagaaaaatggaaggagcccttctctgaatacagctttctgaaatgcccaatgctttgtgaagagtgttgtcttcacgaatccccttgtgactctgataaaagacagaaactcttggcagacatcagagcagatgaagaaattttgagagaaacacgcaggccgggtcggtctcctaggcgcaagtcaccgggaagaagctcgaacccaaagcccaggcctcatgaatgcagcccaccgatgcccaccgcgtcttcccgagggcgggagcaagccatcagaaggagtgaaaaggccaggatgagagaatattggcaagaactggaggaacaagaggaaaaactgcagaagaggccgatgttgtttgaaagagtcacccagagaaatgcaaggatggccgcagagaggtgttactctaacactctaaaagcactaggactccttgaggagtttgtttcagagaaaggccaaagtggaaaagtgtctgaagacttcaccaggcaagagctgagaagctgcacactggagaaagaaagctcttatggagaagaaagagagaatgaggaagaaagctatttaactgatatcagcagccaggattcttgcaagggaaacaaggaagatattaaagatattaaaagtggagaagaaaattctggagaatgagcggccgcctgcaggtcgacggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagaacccagctttcttgtacaaagtggtgataattcctgcagcccgggggatccactagttctagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcctagagcatggctacgtagataagtagcatggcgggttaatcattaactacaaggaacccctagtgatggagttggccactccctcctctgcgcgccaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 29 - DNA, 1728 bp, unidentified
  source 1..1728: mol_type = unassigned DNA
atgtcatcagatgaggagaaatactcacttccagttgtgcaaaatgactccagtcgaggcagttctgtctcttcgaatcttcaggaagaatatgaagaactgcttcattatgctatagtgactccaaatattgaaccctgtgcttcacagtcatctcatcctaagggagaattggtgccagatgtcagaatttctacaattcatgatattcttcatagtcaaggaaataactctgaagtaagagaaactgcaatagaagttggaaaaggatgtgatttccatatttcaagtcattcaaagacagatgagtcatcaccagtgttatcgccaaggaagccttctcacccagtcatggattttttcagttcacatcttttagctgactcttcctcaccagcaacaaattctagtcatacagatgcccatgaaatacttgtgagcgattttcttgtttctgatgaaaaccttcagaagatggaaaatgtgcttgatctttggagttcaggtcttaagacaaacatcatatctgaactaagtaaatggagacttaattttattgactggcaccgaatggaaatgagaaaagagaaagaaaaacatgcagcacatttaaaacaactgtgtaatcagatcaatgaattgaaggagctgcaaaaaacctttgaaatctccattgggagaaaagatgaggtgatttctagcttgtctcatgccataggcaagcaaaaggaaaagatagagttgatgagaacattcttccactggcgaatcggccatgtcagagccagacaggatgtttatgaaggtaaactagctgaccagtactaccagagaactttactgaagaaagtctggaaagtctggcgttccgtagtgcaaaagcagtggaaagatgtggtagaaagagcttgtcaagcaagagctgaagaagtttgtatccagatttccaatgattatgaagccaaagttgctatgttatctggagctttggaaaatgcaaaagctgagattcaaagaatgcaacatgaaaaagagcactttgaagattccatgaaaaaagctttcatgaggggtgtatgtgcattaaatcttgaagccatgactatatttcaaaacagaaatgatgcagggatagactccacaaataataaaaaggaagagtatggtcctggtgttcaaggaaaagaacattctgctcatttggatccttcagctcctccgatgcccttaccagttacatcaccactgctgccatccccaccagccgccgtcggaggagccagcgcgactgccgttccctcagctgcttcgatgacttctaccagggctgcttccgcatcttctgttcacgttcctgtttctgctcttggtgcaggatctgcagctactgctgcatcagaagaaatgtatgtgccaagagttgtaacctctgcacaacagaaagcaggaagaactattacagcccggatcacaggaagatgtgattttgcttcaaaaaatagaattagcagcagtttagctataatgggagtttctcctcccatgagctcagttgttgtggaaaaacatcatccagtcacagtgcaaaccattcctcaagcaactgcagcaaaatatccccggaccattcatcctgaaagtagtacctcagcttccagatcacttggaaccagatcagctcacacccagtctctcacaagtgttcattccataaaagtggttgactaa

SEQ ID NO: 30 - DNA, 4281 bp, unidentified
  source 1..4281: mol_type = unassigned DNA
atgaacgactggcacaggatcttcacccaaaacgtgcttgtccctccccacccacagagagcgcgccagccttggaaggaatccacggcattccagtgtgtcctcaagtggctggacggaccggtaattaggcagggcgtgctggaggtactgtcagaggttgaatgccatctgcgagtgtctttctttgatgtcacctaccggcacttctttgggaggacgtggaaaaccacagtgaagccgacgaagagaccgccgtccaggatcgtctttaatgagcccttgtattttcacacatccctaaaccaccctcatatcgtggctgtggtggaagtggtcgctgagggcaagaaacgggatgggagcctccagacattgtcctgtgggtttggaattcttcggatcttcagcaaccagccggactctcctatctctgcttcccaggacaaaaggttgcggctgtaccatggcacccccagagccctcctgcacccgcttctccaggaccccgcagagcaaaacagacacatgaccctcattgagaactgcagcctgcagtacacgctgaagccacacccggccctggagcctgcgttccaccttcttcctgagaaccttctggtgtctggtctgcagcagatacctggcctgcttccagctcatggagaatccggcgacgctctccgaaagcctcgcctccagaagcccatcacggggcacttggatgacttattcttcaccctgtacccctccctggagaagtttgaggaagagctgctggagctccacgtccaggaccacttccaggagggatgtggcccactggacggtggtgccctggagatcctggagcggcgcctgcgtgtgggcgtgcacaatggtctgggcttcgtgcagaggccgcaggtcgttgtactggtgcctgagatggatgtggccttgacgcgctcagctagcttcagcaggaaagtggtctcctcttccaagaccagctccgggagccaagctctggttttgagaagccgcctccgcctcccagagatggtcggccaccctgcatttgcggtcatcttccagctggagtacgtgttcagcagccctgcaggagtggacggcaatgcagcttcggtcacctctctgtccaacctggcatgcatgcacatggtccgctgggctgtttggaaccccttgctggaagctgattctggaagggtgaccctgcctctgcagggtgggatccagcccaacccctcgcactgtctggtctacaaggtaccctcagccagcatgagctctgaagaggtgaagcaggtggagtcgggtacactccggttccagttctcgctgggctcagaagaacacctggatgcacccacggagcctgtcagtggccccaaagtggagcggcggccttccaggaaaccacccacgtccccttcgagcccgccagcgccagtacctcgagttctcgctgccccgcagaactcacctgtgggaccagggttgtcaatttcccagctggcggcctccccgcggtccccgactcagcactgcttggccaggcctacttcacagctaccccatggctctcaggcctccccggcccaggcacaggagttcccgttggaggccggtatctcccacctggaagccgacctgagccagacctccctggtcctggaaacatccattgccgaacagttacaggagctgccgttcacgcctttgcatgcccctattgttgtgggaacccagaccaggagctctgcagggcagccctcgagagcctccatggtgctcctgcagtcctccggctttcccgagattctggatgccaataaacagccagccgaggctgtcagcgctacagaacctgtgacgtttaaccctcagaaggaagaatcagattgtctacaaagcaacgagatggtgctacagtttcttgcctttagcagagtggcccaggactgccgaggaacatcatggccaaagactgtgtatttcaccttccagttctaccgcttcccacccgcaacgacgccacgactgcagctggtccagctggatgaggccggccagcccagctctggcgccctgacccacatcctcgtgcctgtgagcagagatggcacctttgatgctgggtctcctggcttccagctgaggtacatggtgggccctgggttcctgaagccaggtgagcggcgctgctttgcccgctacctggccgtgcagaccctgcagattgacgtctgggacggagactccctgctgctcatcggatctgctgccgtccagatgaagcatctcctccgccaaggccggccggctgtgcaggcctcccacgagcttgaggtcgtggcaactgaatacgagcaggacaacatggtggtgagtggagacatgctggggtttggccgcgtcaagcccatcggcgtccactcggtggtgaagggccggctgcacctgactttggccaacgtgggtcacccgtgtgaacagaaagtgagaggttgtagcacattgccaccgtccagatctcgggtcatctcaaacgatggagccagccgcttctctggaggcagcctcctcacgactggaagctcaaggcgaaaacacgtggtgcaagcacagaagctggcggacgtggacagtgagctggctgccatgctactgacccatgcccggcagggcaaggggccccaggacgtcagccgcgagtcggatgccacccgcaggcgtaagctggagcggatgaggtctgtgcgcctgcaggaggccgggggagacttgggccggcgcgggacgagcgtgttggcgcagcagagcgtccgcacacagcacttgcgggacctacaggtcatcgccgcctaccgggaacgcacgaaggccgagagcatcgccagcctgctgagcctggccatcaccacggagcacacgctccacgccacgctgggggtcgccgagttctttgagtttgtgcttaagaacccccacaacacacagcacacggtgactgtggagatcgacaaccccgagctcagcgtcatcgtggacagtcaggagtggagggacttcaagggtgctgctggcctgcacacaccggtggaggaggacatgttccacctgcgtggcagcctggccccccagctctacctgcgcccccacgagaccgcccacgtccccttcaagttccagagcttctctgcagggcagctggccatggtgcaggcctctcctgggttgagcaacgagaagggcatggacgccgtgtcaccttggaagtccagcgcagtgcccactaaacacgccaaggtcttgttccgagcgagtggtggcaagcccatcgccgtgctctgcctgactgtggagctgcagccccacgtggtggaccaggtcttccgcttctatcacccggagctctccttcctgaagaaggccatccgcctgccgccctggcacacatttccaggtgctccggtgggaatgcttggtgaggaccccccagtccatgttcgctgcagcgacccgaacgtcatctgtgagacccagaatgtgggccccggggaaccacgggacatatttctgaaggtggccagtggtccaagcccggagatcaaagacttctttgtcatcatttactcggatcgctggctggcgacacccacacagacgtggcaggtctacctccactccctgcagcgcgtggatgtctcctgcgtcgcaggccagctgacccgcctgtcccttgtccttcgggggacacagacagtgaggaaagtgagagctttcacctctcatccccaggagctgaagacagaccccaaaggtgtcttcgtgctgccgcctcgtggggtgcaggacctgcatgttggcgtgaggccccttagggccggcagccgctttgtccatctcaacctggtggacgtggattgccaccagctggtggcctcctggctcgtgtgcctctgctgccgccagccgctcatctccaaggcctttgagatcatgttggctgcgggcgaagggaagggtgtcaacaagaggatcacctacaccaacccctacccctcccggaggacattccacctgcacagcgaccacccggagctgctgcggttcagagaggactccttccaggtcgggggtggagagacctacaccatcggcttgcagtttgcgcctagtcagagagtgggtgaggaggagatcctgatctacatcaatgaccatgaggacaaaaacgaagaggcattttgcgtgaaggtcatctaccagtga

SEQ ID NO: 31 - DNA, 561 bp, unidentified
  source 1..561: mol_type = unassigned DNA
atgggattgctagacagactttcagtcttgcttggcctgaagaagaaggaggttcatgttttgtgccttgggctagataatagtggcaaaacgacgatcattaacaaacttaaaccttcaaatgctcaatctcaaaatatccttccaacaataggattcagcatagagaaattcaaatcatccagtttgtcatttacagtgtttgacatgtcaggtcaaggaagatacagaaatctctgggaacactattataaagaaggccaagctattatttttgtcattgatagtagtgatagattaagaatggttgtggccaaagaagaactcgatactcttctgaatcatccagatattaaacaccgtcgaattccaatcttattctttgcaaataaaatggatcttagagatgcagtgacatctgtaaaagtgtctcagttgctgtgtttagagaacatcaaagataaaccctggcatatttgtgctagtgatgccataaaaggagaaggcttgcaagaaggtgtagactggcttcaagatcagatccagactgtgaagacatga

SEQ ID NO: 32 - DNA, 2663 bp, unidentified
  source 1..2663: mol_type = unassigned DNA
atggccgctgcgtcctcatcggattccgacgcctgcggagctgagagcaatgaggccaattcgaagtggttggatgcgcactacgacccaatggccaatatccacaccttttctgcctgcctagcgctggcagatttacatggggatggggaatacaagctggtggtaggggaccttggccctggtgggcagcagccccgcctgaaggtgctcaaaggaccactggtgatgaccgaaagcccgctacctgctctgccagctgctgctgccaccttcctcatggagcaacatgagccccggaccccagctctggcacttgcttcaggcccttgtgtctatgtgtataagaatctcagaccctacttcaagttcagcctgccccaattgcctccaaatcctctggaacaagacctttggaaccaggccaaagaggaccgaatcgaccccttaaccctgaaggagatgctggagagcatccgggagacggcagaggagcctttgtccatccagtcactcaggtttctgcagctggagctaagtgaaatggaggcatttgtaaaccaacacaagtccaactccatcaagcggcagacagtcatcaccaccatgaccaccttgaagaagaacctggctgacgaggatgctgtgtcttgcctggtgctgggcaccgagaacaaggagctcctggtgcttgaccccgaggccttcaccattttagccaagatgagccttcccagcgtccccgtcttcctagaggtttctggccagtttgatgttgagttccggcttgccgcggcctgccgcaatggaaacatctatattctgagaagagactccaagcaccccaagtactgcatcgagctgagcgcccagcctgtgggacttatccgggtacacaaggtcctagtggtgggcagcacccaagacagcctgcatggcttcacccacaaggggaagaagctgtggacagtgcagatgcccgcagccatcctgaccatgaacctcctggagcagcattcccggggcctgcaggccgtcatggctgggctggccaatggagaggtccgcatttatcgtgacaaggccctgctcaatgtcatccacaccccggatgcagtgaccagcctttgctttggccggtacgggcgggaggacaacaccctcatcatgaccactcgaggtggtggcctgatcatcaagatcctgaagcgtacagcagtgtttgtagagggaggaagtgaggtgggtcccccaccagcccaggccatgaaactcaatgtgccccgaaagacccggctttacgtggatcagacactgcgagagcgggaggctggcaccgccatgcaccgggccttccagacagacctatacctgctgcgcctacgtgctgcccgcgcctacctgcaggccctcgagtccagcctgagccccctgtccacgacagcccgagagccactcaagctgcacgccgtggttcagggccttggccccacctttaagctcacacttcacctgcagaacacctcaacaacccgtcctgtcctggggctgctggtctgcttcctgtacaacgaggcgctctattccctgccccgggccttcttcaaggtacccttgctggtgccagggctcaactaccccctggagacctttgtggagagtctcagtaacaagggcatctcagacatcatcaaggtgctggtgcttcgagaaggccaaagtgcacccctgctgagtgcccacgtcaacatgcctgggagcgaggggctggcggccgcctgagacctgagctgctgtgaaagcccctgcacaatcagccagggagaactgggcgggtttagtggccccaggcccactcctcatgcagcagtgtgctggggcgacagctcgtctcccctctcttaagcacccgcttcctcaccacccccactgttgggcctatagtagcaggttagtgagtacctagggcggctcaactcctcccacagcaccaacccagcatggtcccactgaagtcctactacgccctcccctccccagccttttccagaaaccatactgggctcagatcagagctccgaagcggtcaaagtgagctgagcaggacaggcccagcctttctccactgccacgtccctcatgcacatcactcatctcctgctgcaggccaaggccaaaattgggctagtcctggccagggaaatcagaagctcttcttgggtgagattgagcctcctgttgctccctggagttccggaggctgggctgcagcccactcagcttgcgggcaaaatacgtgctctcctctctccttgtcagctgagcaaacccagggaatagccctcctctccccaggaaacttctctgaaatcttagacttagccagtcttaggcctacgatgccacacaaaggttgttcagggagaagggggtgcaggaggcagagggtgccccgcagggagctggtggctccagccccactagagctcctaaagatcacacagcagctgctcctgacagggatgctcatgcccagaaagcaagcccaggagaggaaggcagagtgtgacagagcagagccagggccaggcgcaccaggagaggcgtttctggggctccagagaagtgccacgggaggcagaagtccagaactgcccatatag

SEQ ID NO: 33 - DNA, 2166 bp, unidentified
  source 1..2166: mol_type = unassigned DNA
atgctgctgcctgtgttcaccctgaaactgcgccacaaaatcagcccccgaatggtggccatagggcgctacgacgggactcacccgtgcctggcggccgccacccaaacgggcaaggtttttattcataatcctcatacacggaaccagcatgtcagtgcatccagggtcttccagagccccctggaatctgatgtttctcttctcagcattaaccaggcagtcagctgtctgactgcaggcgtattgaaccctgagcttggctatgatgcccttttagtggggacacagactaatcttttggcttatgatgtctacaataattcggatttgttctacagagaggtagcagatggggcaaatgcaattgtgctggggacattgggagacatttcttcccctcttgcgattattggtggcaattgtgctctgcaaggtttcaatcatgaaggaagtgatctcttttggacggttactggagacaatgttaattccttggccttgtgtgactttgatggtgatggaaagaaagagcttcttgttggatctgaggattttgatatccgagtttttaaggaagatgagattgtggcagaaatgacagaaacagagatagtcacctctctttgtcccatgtatggcagtcgatttggttatgccctttccaatggcacagttggagtttatgacaaaacatcccgatactggagaattaaatcgaaaaatcatgccatgagcattcatgcttttgaccttaattctgatggagtgaatgaactgataactggttggtccaatgggaaggttgatgctcgaagtgaccgaactggggaggtcatctttaaggacaatttttcttctgcaattgccggtgtggtagagggagattaccggatggatggccacatacagttaatctgctgctcagtggatggggaaatccggggctacctgcctggcacggctgagatgaggggcaacctcatggacaccagtgcagagcaggacctgatccgagagctgagtcagaagaagcagaatctgttgctggaactccgtaactatgaggaaaatgccaaggctgaattggccagtccactgaacgaggctgatgggcatcggggcataatcccagccaataccaggctccacaccacgctctcagtcagcctggggaatgagacccaaactgctcatacagaattacgcatttccacttctaatgacaccatcatccgagcagtattgatttttgcagaaggaatttttacaggtgaaagccacgtggtacatcccagcattcacaacctctccagttccatctgcatccctattgtgcctcccaaagatgtccctgtggatctgcacttgaaggcattcgtgggttacagaagcagcacccagtttcatgtatttgaatcgacaagacagctccctcgattctccatgtatgcgctgaccagcctggaccctgccagtgagccaatcagttatgttaactttaccattgcagaacgggcacagagggttgttgtatggctcggtcagaactttctgttaccagaagacactcacattcagaatgctccatttcaagtgtgtttcacatctttacggaatggcggccacctgcatataaaaataaaacttagtggagagatcactataaatactgatgatattgatttggctggtgatatcatccagtcaatggcatcattttttgctattgaagaccttcaagtagaagcggattttcctgtctattttgaggaattacgaaaggtgctagttaaggtggatgaatatcattcagtgcatcagaagctcagtgctgatatggctgatcattctaatttgatccgaagtttgctggtcggagctgaggatgctcgtctgatgagggacatgaaaacaatgaagagtcgttatatggaactctatgaccttaatagagacttgctaaatggatataaaattcgctgtaacaatcacacagagctgttgggaaacctcaaagcagtaaatcaagcaattcaaagagcaggtcgtctgcgggttggaaaaccaaagaaccaggtgatcactgcttgtcgggatgcaattcgaagcaataacatcaacacactgttcaaaatcatgcgagtggggacagcttcttcctag

SEQ ID NO: 34 - DNA, 2664 bp, unidentified
  source 1..2664: mol_type = unassigned DNA
atgtctttatttaaagcccgtgattggtggtctactattctgggagataaagaagaatttgatcaaggctgtttgtgtctggctaatgttgacaatagtggaaatggacaagataaaataattgtgggtagctttatgggatacctaaggatctttagcccccatcctgcaaaaacaggagatggagctcaagccgaagatttgcttctagaagtggatctacgagatccagtacttcaagtggaagtaggaaagtttgtttcaggtaccgaaatgctacatttggctgtgttacattctagaaaactttgtgtctactctgtctcaggaaccttgggtaatgtggaacatgggaaccaatgtcagatgaaattgatgtatgaacataatcttcagagaacagcctgcaatatgacctatggatcatttggtggtgtaaaaggtcgagatttaatttgcatccagtctatggatgggatgctgatggtatttgagcaggagagctatgcttttggaagatttctccctggctttcttctgcctggtcctcttgcctacagttcccgtacagattccttccttactgtctcttcctgccaacaagtggaaagttataagtaccaggtacttgcttttgcaacagatgcagataaaaggcaggagactgaacagcaaaaacttggttctggaaaaagactagttgtggattggactctaaatattggagagcaagcccttgacatatgtattgtctctttcaatcagtcggcatcctctgtttttgttcttggtgagagaaactttttttgccttaaggataatggacaaattcgattcatgaagaagcttgattggagcccaagttgttttctgccatattgctcagtttctgaaggaacaataaatactttgattggaaatcataataacatgctgcatatttatcaagatgtgacactgaagtgggccacccaacttccccacattcctgtagcagtaagagtgggctgtttgcatgatttaaagggagtgatagtcactctgagtgatgatggtcacttgcagtgttcatacctggggacagatccttctctgttccaagctccaaacgttcaatctcgagaactaaactatgatgaacttgatgtagaaatgaaagaacttcagaaaatcatcaaagatgttaacaaatcacaaggtgtttggcccatgactgagagagaagatgacttgaacgtttctgtcgtggtttctcctaactttgattcagtttctcaagcgaccgatgttgaggtgggaactgaccttgtcccttctgtcacggtgaaggtcacactgcagaacagagtgatattgcaaaaagccaaattatcagtctacgtgcaaccaccattagaattgacttgtgatcagttcacctttgaatttatgacaccagatttgactagaacagtaagcttttctgtttatctgaaaagaagttatacaccatcagaattggaaggaaatgctgttgtttcttattccagaccaacagatcgaaatcctgatggcattccgcgagttatccaatgtaaatttagacttcccctaaagttaatttgcctaccaggtcagccttcaaaaactgcaagccacaaaattactattgataccaacaaatctccagtcagtcttcttagtctcttcccaggttttgccagtcagtcagatgatgatcaggtgaatgtaatgggttttcacttcttaggaggtgctcgaattactgttcttgcttccaaaacttctcaacgatatcgcattcagagtgaacaatttgaagatctttggctcataaccaatgagcttattcttcgccttcaagaatattttgaaaaacagggagtcaaagattttgcatgttctttttcgggatctataccccttcaagaatattttgagttgattgatcatcattttgagctacggataaatggtgaaaaattagaagaactcttatctgagagagctgtacaatttcgggccattcaacgccggctactagcaagattcaaagataaaactcctgcccctcttcaacacctggacaccttgttagatggaacctacaagcaggtaattgctctagcagatgcagtggaggaaaaccaaggcaatctgttccagtcattcaccaggctgaagagtgccacccatttggtgattctgctgatcgcgctgtggcagaagcttagtgctgaccaggttgctattctggaagcggcatttctgccgctacaagaagacactcaagaattgggctgggaagaaacggtggatgccgccatttcccacctgttgaagacttgcctgtcgaagagttctaaggagcaggctttgaacctcaacagccagctgaacatacccaaagacacaagccaactgaagaaacatatcaccttgctctgcgatagattatccaaaggtggccgtctctgcctaagtaccgatgcagcagccccacagaccatggtcatgccaggtggttgtactacaatcccagagtcagacctagaagaaagatcagtagaacaagactctacagaactgtttaccaaccacagacatctcactgcagagacacccaggcctgaagtttcacccctccaaggagtctcggaataa

SEQ ID NO: 35 - DNA, 3867 bp, unidentified
  source 1..3867: mol_type = unassigned DNA
atggggtgtacaccttcacacagtgaccttgtaaacagcgttgcaaagagtggcattcagttcttgaaaaagcccaaagcaattcggccaggatgtcagggcggaagtgaaagaggttccatccctttgctggttaaaaactccacctgctatgacgctggggagggcctggcagaggagcagccaagtcccaggaggaaccaaaccacagctaaaggtctttgtcagctcatgggagatcctgcttcaggcaaaaggaaagatatggaaggactgatcccaggaaccaaaacctcttcatcccagctgaacaaatcacaaagccacatggctaaggatattccgttcaagacacagggttcccatggatcacaaggggcagacttttctggagatgagagtgaggaaagtagtacccaagatacttccaaatggaaaaggacagcaaaatgtcacacgtcaagcacacagagccactgctaccaaaccatccaccctgctcatgagcctgaaggcaaagtggacttcccggagcctctggtaaaggcccaccagcaggcttacacctatctacactccagcctctccaaatatgaagcaattctgtgcatcatccatcaggccacccagacccgggagctgctgcagcccatggtcagcttcttgctgctgtgctttgaggagatcagccagctgttgggggagatctccaaggatggagaagtgctcctgcaggaagtcagggaggatctggcttggcctttgaagaaaagagagccccaggagcagccaaatctcctgcaacagctgctacagtacacagtcagcaagctgcaggtgctcaatggcacagtggcctcgctcaccggcagcttcctggagggctccagcagctacctccactccactgcaacccacttggaaaataagctgagcacaaaaaggaatgtggatgaacgcctcctgagggctctgaggcagctagagagcctggcgagtggctgtggcgaccctggggtgcagggtctccccttatgctctgaggacagtggcattggtgctgacaatgagtccgtgcagtcggtggacaagctgggcaagcaaaccagctgggaccttgcaccagagcccgaagaatggaagtcggtgacttcaccccacacagaggccaggcagtcaggacacacctggcagcaaagtccattctgtttgggctcaggcagaccccaggactgcctgctctcaggggctcctatggcaaaggttcagccacgagcacaggacgaagcaaggagcccatgcctctccagtacaagcccagaaaatatcacctccccacctttgaagctggggacaagcaccccatgtgattcctttgggattggggtctctgtggaaccacacctttccaaaacctccaggccgatggacgcttcatctcttagtgacagcgaggacagcagcccagaggaggaggaggaagacaaaatgagcagcatgagtctgtgtgcctggcaggaaaaaactccacattcaaggccacaatcttcacctgctgaccgggaaagcccatttcaggcccgcaccaggaggcttaggagcctccaggcccaggaaatgattctgaagatgaaggagtcaatcagcgaaaggatcaagtttgtccctgtgccctgtgggcaccaggactggtctgaggaggaggaggggaggacagtggtccccccaagacctagcacggtaagtggcagcaggagggcccctgagaggcagacgaggtcccagtcagagtcgtgtctccagagtcacgtggaggaccccacctttcaggagctgcgaagggtccagagggacctcagtcagaagctggaggcattttatgccctgggtgccaaagggcaggggcagagccaggagcaaattctgcagcccagagcagccgccgtgtggcccaatggcacctgcagggtcagtccaagcaacaccaccagcaggctcaaggcatccctcaccaagaacttcagtattttgcctagtcaggacaagagcatcttgcagaaatgcaatccccatcctgaggacgaacaaggcaaagctgggaagcttccaaatgccatcccatcaggagaggtcagtgaggctgccaaggccacagactggaatgtcagaggctgtcccaccagaacatccgtcaagaagcttattgaaactttcagtcccacggagagtctgaggatgctgggggactctaaggacgctggggcaagtccctgcctcaggaattgcatcatgccccccagatttcccaagtacacagggcttgcccctttgtatccgaagccccaaatttctccagcatcaggcagagaatctctcaaaatgggcataggctggaagcccttagcacctatctttccccctctgcctaaagcagaagcagccaagagtgaggagctcagctgtgaaatggaggggaacctcgagcacctccctccaccgcctatggaagttctgatggacaaatcattcgcttctctggagtccccagaaagcagcaagtccacagagaactcccccaaggaaacccaggagccagggccgggagaggctggccccaccaggagaacatgggcttccccaaagctgagggcctctgtgagccccctggacttgctgcccagcaagagcaccgccagcctgaccaagcctcacagcacagggccagggagtggcaggagcagctgccagcccaggaagccagccctggacctgagcagcccaccagccaccagccaaagccccgaggtgaagggtgggacttggagtcaggcagagaaggccaccagcctctacaggcagccccggaaggccatcgcctggcaccactccggccctccatctggacaaaacaggacctcagagtccagcctggccagaccaaggcagagccgagagagaagcccccctgtgggcagaaaggcctctcccacgaggacacactgggtgcctcaagcagacaagaggcgccggagccttccctcctcttacagacctgcccagccaagcccctctgctgtgcagacgccccccagcccacctgtgagccccagggtgctaagcccacccaccacaaagcggcgaacttccccaccgcaccagcccaagttgcccaaccctccccccgagagtgcacctgctcagtgcaaggtccccagccccccaacccagcacccagaagcaagcccccctttctcgattccctccccatcacccccaatgtccccttctcaggagcacaaggaaacaagagactctgaagacagccaagcagtcatagccaaagtgtctgggaacacacattccatattctgcccagctacctcctctctgtttgaagctaaaccgccactctcaacagcccacccactgaccccaccatcgctgccgccagaggctgggggccctctcgggaacccagcagaatgctggaagaacagctcagggccttggctgagagcagactcgcagcggagagcagctctgtgtgccctcaaccctctgcctttcctcaggaggacagcttctgaccgccagccaggtggccgaccgcagcctcccaccttggaccccaccagcacctcttatgaatcccagctcggccagaacagcagcagcgaggagagccctaagaaggacacagagccggggagcagcccctgttcccctgaactgcagggcggcaccaggcgtgcatctcccccagagttctgtgtgctgggccacgggctgcaaccggagcctcggaccggccacatccaggacaaatcccagccagaggcgcagccccagcaagaggaggtgtcctga

SEQ ID NO: 36 - DNA, 624 bp, unidentified
  source 1..624: mol_type = unassigned DNA
atggcggaggacctggacgagctcttggatgaagtcgagtccaagttttgcacacctgaccttctaagacggggtatggtcgagcagcccaaaggctgcggcggcggcacccacagtagcgaccggaaccaagccaaggcgaaagagacgctcagatcaacagaaacatttaaaaaagaagatgatcttgacagtcttattaatgaaatacttgaagagcccaacttggacaaaaaaccctctaaattaaaatctaaatcttcaggtaacacatctgtcagagcttccattgaaggccttggtaaaagttgcagtccggtgtaccttggtggaagctctattccatgtgggattggaacaaatatttcatggagagcatgtgaccatctgcgttgtatagcctgtgatttcttggtagtcagctatgatgactatatgtgggacaaatcgtgtgattatctgtttttcaggaacaacatgccagaatttcacaaattaaaagcaaagttgataaagaagaaaggaacacgggcatatgcctgccagtgtagctggagaactattgaagaagtgactgaccttcagacagatcatcagcttcgctgggtttgtggtaaacattaa

SEQ ID NO: 37 - DNA, 4383 bp, unidentified
  source 1..4383: mol_type = unassigned DNA
atggctggacgacccctccgcataggagatcagctggttctggaagaagattatgatgagacctacattcctagtgagcaagaaattcttgaatttgcccgggagattggtattgatcccatcaaggaaccagaactgatgtggctggcgcgagagggcatcgtggccccactgcctggagagtggaaaccatgccaggacatcacaggtgacatttactatttcaacttcgccaacgggcagtctatgtgggaccatccatgtgacgaacactatcggagcttggtgatccaagagcgggcaaagctgtcaacttctggggccattaagaagaagaaaaaaaaaaaggaaaagaaagacaagaaggacagagacccccccaaaagttcgctggccttgggttcctcattagccccagttcatgttcctcttgggggcctggctcctttacgaggtcttgtggataccccaccctctgctcttcgtggatctcaaagcgtgagcctggggagctcagtggagtctggacgtcagcttggagaactcatgctgccttcacagggtctcaagacctctgcttatacaaagggtctcttgggctccatatatgaggacaagactgctctcagcctcttgggtttaggagaagaaaccaatgaggaggatgaggaggaaagtgacaaccagagtgtccacagctcaagtgagcctcttaggaacctacacctggacattggggcactggggggtgactttgagtatgaggagtctctgagaacaagccagccagaggagaagaaggatgtttctctggattcagatgctgccggtccccctactccctgcaagccctccagcccaggtgcagacagcagtctgagcagtgctgttggcaaagggcgacagggaagtggagcaagacctggtcttccagaaaaagaggaaaatgagaagagtgaacctaagatttgcaggaatctggtgacccccaaggcagaccctacaggcagtgagcctgccaaagcctctgaaaaggaagcaccagaggacacagtagatgcaggagaggagggttccaggagggaagaggcagccaaggagccaaagaagaaggcttctgctctggaagagggcagttcagacgccagccaagaactggaaattagtgaacacatgaaggaaccacagctctcagactccatagcttctgaccccaagtccttccatggcctggacttcggttttcgcagccggatctcggagcacctgctggatgttgatgtgctttccccagtcctgggtggagcttgtcggcaggcccagcaaccactgggaatagaagacaaggatgacagccagtccagccaagatgagctgcagagcaagcagtccaaaggcctggaggagaggttatctcctccacttccacacgaggagcgggcccagagtccccctcgcagcctggccactgaagaagagcctccccagggccccgaggggcagcccgagtggaaggaggcagaggagcttggggaggactctgcagccagcctcagcctgcagctgtccctccagagggagcaggccccaagcccacctgctgcctgtgagaagggcaaggagcagcattcccaggccgaggagctgggccctgggcaggaagaggcagaggatcctgaggagaaggtggcggtcagccccaccccgccagtctctccagaggtgcgatccacagagcctgtggctcccccagagcagctctcagaggctgcactaaaggccatggaagaggcagtggcccaagtactcgagcaagaccagaggcacctgctggaatccaagcaagagaagatgcagcaactgcgggagaagctgtgccaagaggaggaagaggagatcctccggcttcaccagcagaaagagcaatctctcagttccttgagggagcggctgcagaaagccattgaggaggaggaggcccggatgagagaggaggaaagccagaggctatcctggctccgagctcaggtccagtccagcacacaagcagatgaggaccaaatcagggctgagcaagaggcttccctgcagaaactgagagaagagttggagtctcaacagaaggctgagagggccagcttggaacagaaaaataggcaaatgctggagcagctcaaggaagagatagaggcttcggagaagagcgagcaggctgccctgaatgctgcaaaggagaaggctctgcagcagctgagggagcagctggaaggggagaggaaagaagctgtggcaacgctggagaaggagcacagtgctgagctggagcggctctgctcctcattggaggccaagcaccgggaggtggtctccagcctccagaagaagatacaggaagctcaacagaaagaggaggcccagctgcagaagtgccttgggcaagtggagcacagagttcaccagaagtcttatcacgtggctgggtatgagcacgagctcagcagtctcctgcgagagaagcgccaggaagtggaaggggagcatgagaggaggttggacaagatgaaggaggagcaccagcaagtgatggctaaggccagagagcagtatgaagctgaggagaggaagcagcgggctgagcttctggggcacctgaccggagagctggagcgcctgcagagggcccatgaacgagaactggagactgtgaggcaggagcaacacaagcgtcttgaggacttgcggcgccggcacagggagcaggaaaggaagctccaggatttagagttggaccttgaaaccagagctaaagatgtcaaggccagattggctctgctggaggtccaggaggagaccgcccggagggagaagcagcagctgcttgatgtgcagaggcaggttgctctgaagagtgaggaagccacagccacccatcagcagctggaggaggcacagaaggagcacacccacctgttgcagtcaaaccagcagctccgagaaattcttgatgagctgcaggcccgcaagctgaagctggagtcccaagtggatctgctgcaggctcagagccagcaactgcagaaacacttcagcagcctggaggctgaagctcaaaagaagcagcacctgttgagagaagtgacagttgaggaaaataatgcttccccacattttgagccagatctccatattgaggacctgaggaaatcccttggaacaaaccagaccaaagaggtgtcttcttctctctcccagagcaaggaggacttatacttggacagcctgtcctcccacaatgtctggcacctcctctctgctgagggggtagccctccgtagtgccaaggagttccttgtgcagcagacacgctccatgcggaggcggcagacagctctgaaagctgcccagcagcattggcgccatgagctggccagtgcgcaggaggtggccaaagacccaccaggcatcaaggccctggaagatatgcgcaagaacctggagaaggagaccaggcacctggatgagatgaagtcggccatgcggaaaggccacaacctgctgaagaagaaagaggagaagctgaatcagttggagtcctctctttgggaagaggcctcagatgagggcactctgggaggatcccccaccaagaaggcagtaaccttcgacctcagtgacatggacagcctgagcagtgaaagttctgaatctttttccccgcctcaccgtgagtggtggcggcagcagaggatcgactcaaccccgagtctcacctcccgcaagatccacgggcttagccactccctccggcagatcagcagccagctgagcagtgtcctcagcatcctggacagcctcaaccctcagtcgccgccgccgctcctcgcctccatgccagcccagctccctccccgggaccctaagagcacccccacccccacctactatggctccctggccaggttctcagccttatcatctgctacacccacgtccacccaatgggcctgggattcagggcaggggcccaggctcccctcctctgtggctcaaacggtggacgacttcctgttggagaagtggcgcaagtattttccatctggcatcccgctgctcagcaacagccccaccccgctggagagcaggctgggttacatgtctgccagtgagcagctccggctcctacagcactcccattcgcaagtccctgaggcgggcagcaccacctttcagggcataattgaggccaaccggaggtggctggaacgtgtcaagaatgaccccaggttacctctcttctcgtcaacacccaagccaaaagctactttgagcctcctgcagctgggccttgatgagcacaacagagtgaaggtgtatcgcttctga

SEQ ID NO: 38 - DNA, 4368 bp, unidentified
  source 1..4368: mol_type = unassigned DNA
atggctggacgacccctccgcataggagatcagctggttctggaagaagattatgatgagacctacattcctagtgagcaagaaattcttgaatttgcccgggagattggtattgatcccatcaaggaaccagaactgatgtggctggcgcgagagggcatcgtggccccactgcctggagagtggaaaccatgccaggacatcacaggtgacatttactatttcaacttcgccaacgggcagtctatgtgggaccatccatgtgacgaacactatcggagcttggtgatccaagagcgggcaaagctgtcaacttctggggccattaagaagaagaaaaaaaaaaaggaaaagaaagacaagaaggacagagacccccccaaaagttcgctggccttgggttcctcattagccccagttcatgttcctcttgggggcctggctcctttacgaggtcttgtggataccccaccctctgctcttcgtggatctcaaagcgtgagcctggggagctcagtggagtctggacgtcagcttggagaactcatgctgccttcacagggtctcaagacctctgcttatacaaagggtctcttgggctccatatatgaggacaagactgctctcagcctcttgggtttaggagaagaaaccaatgaggaggatgaggaggaaagtgacaaccagagtgtccacagctcaagtgagcctcttaggaacctacacctggacattggggcactggggggtgactttgagtatgaggagtctctgagaacaagccagccagaggagaagaaggatgtttctctggattcagatgctgccggtccccctactccctgcaagccctccagcccaggtgcagacagcagtctgagcagtgctgttggcaaagggcgacagggaagtggagcaagacctggtcttccagaaaaagaggaaaatgagaagagtgaacctaagatttgcaggaatctggtgacccccaaggcagaccctacaggcagtgagcctgccaaagcctctgaaaaggaagcaccagaggacacagtagatgcaggagaggagggttccaggagggaagaggcagccaaggagccaaagaagaaggcttctgctctggaagagggcagttcagacgccagccaagaactggaaattagtgaacacatgaaggaaccacagctctcagactccatagcttctgaccccaagtccttccatggcctggacttcggttttcgcagccggatctcggagcacctgctggatgttgatgtgctttccccagtcctgggtggagcttgtcggcaggcccagcaaccactgggaatagaagacaaggatgacagccagtccagccaagatgagctgcagagcaagcagtccaaaggcctggaggagaggtaccataggttatctcctccacttccacacgaggagcgggcccagagtccccctcgcagcctggccactgaagaagagcctccccagggccccgaggggcagcccgagtggaaggaggcagaggagcttggggaggactctgcagccagcctcagcctgcagctgtccctccagagggagcaggccccaagcccacctgctgcctgtgagaagggcaaggagcagcattcccaggccgaggagctgggccctgggcaggaagaggcagaggatcctgaggagaaggtggcggtcagccccaccccgccagtctctccagaggtgcgatccacagagcctgtggctcccccagagcagctctcagaggctgcactaaaggccatggaagaggcagtggcccaagtactcgagcaagaccagaggcacctgctggaatccaagcaagagaagatgcagcaactgcgggagaagctgtgccaagaggaggaagaggagatcctccggcttcaccagcagaaagagcaatctctcagttccttgagggagcggctgcagaaagccattgaggaggaggaggcccggatgagagaggaggaaagccagaggctatcctggctccgagctcaggtccagtccagcacacaagcagatgaggaccaaatcagggctgagcaagaggcttccctgcagaaactgagagaagagttggagtctcaacagaaggctgagagggccagcttggaacagaaaaataggcaaatgctggagcagctcaaggaagagatagaggcttcggagaagagcgagcaggctgccctgaatgctgcaaaggagaaggctctgcagcagctgagggagcagctggaaggggagaggaaagaagctgtggcaacgctggagaaggagcacagtgctgagctggagcggctctgctcctcattggaggccaagcaccgggaggtggtctccagcctccagaagaagatacaggaagctcaacagaaagaggaggcccagctgcagaagtgccttgggcaagtggagcacagagttcaccagaagtcttatcacgtggctgggtatgagcacgagctcagcagtctcctgcgagagaagcgccaggaagtggaaggggagcatgagaggaggttggacaagatgaaggaggagcaccagcaagtgatggctaaggccagagagcagtatgaagctgaggagaggaagcagcgggctgagcttctggggcacctgaccggagagctggagcgcctgcagagggcccatgaacgagaactggagactgtgaggcaggagcaacacaagcgtcttgaggacttgcggcgccggcacagggagcaggaaaggaagctccaggatttagagttggaccttgaaaccagagctaaagatgtcaaggccagattggctctgctggaggtccaggaggagaccgcccggagggagaagcagcagctgcttgatgtgcagaggcaggttgctctgaagagtgaggaagccacagccacccatcagcagctggaggaggcacagaaggagcacacccacctgttgcagtcaaaccagcagctccgagaaattcttgatgagctgcaggcccgcaagctgaagctggagtcccaagtggatctgctgcaggctcagagccagcaactgcagaaacacttcagcagcctggaggctgaagctcaaaagaagcagcacctgttgagagaagtgacagttgaggaaaataatgcttccccacattttgagccagatctccatattgaggacctgaggaaatcccttggaacaaaccagaccaaagaggtgtcttcttctctctcccagagcaaggaggacttatacttggacagcctgtcctcccacaatgtctggcacctcctctctgctgagggggtagccctccgtagtgccaaggagttccttgtgcagcagacacgctccatgcggaggcggcagacagctctgaaagctgcccagcagcattggcgccatgagctggccagtgcgcaggaggtggccaaagacccaccaggcatcaaggccctggaagatatgcgcaagaacctggagaaggagaccaggcacctggatgagatgaagtcggccatgcggaaaggccacaacctgctgaagaagaaagaggagaagctgaatcagttggagtcctctctttgggaagaggcctcagatgagggcactctgggaggatcccccaccaagaaggcagtaaccttcgacctcagtgacatggacagcctgagcagtgaaagttctgaatctttttccccgcctcacctcgactcaaccccgagtctcacctcccgcaagatccacgggcttagccactccctccggcagatcagcagccagctgagcagtgtcctcagcatcctggacagcctcaaccctcagtcgccgccgccgctcctcgcctccatgccagcccagctccctccccgggaccctaagagcacccccacccccacctactatggctccctggccaggttctcagccttatcatctgctacacccacgtccacccaatgggcctgggattcagggcaggggcccaggctcccctcctctgtggctcaaacggtggacgacttcctgttggagaagtggcgcaagtattttccatctggcatcccgctgctcagcaacagccccaccccgctggagagcaggctgggttacatgtctgccagtgagcagctccggctcctacagcactcccattcgcaagtccctgaggcgggcagcaccacctttcagggcataattgaggccaaccggaggtggctggaacgtgtcaagaatgaccccaggttacctctcttctcgtcaacacccaagccaaaagctactttgagcctcctgcagctgggccttgatgagcacaacagagtgaaggtgtatcgcttctga

SEQ ID NO: 39 - DNA, 7440 bp, unidentified
  source 1..7440: mol_type = unassigned DNA
atgccacctaatataaactggaaagaaataatgaaagttgacccagatgacctgccccgtcaagaagaactggcagataatttattgatttccttatccaaggtggaagtaaatgagctaaaaagtgaaaagcaagaaaatgtgatacaccttttcagaattactcagtcactaatgaagatgaaagctcaagaagtggagctggctttggaagaagtagaaaaagctggagaagaacaagcaaaatttgaaaatcaattaaaaactaaagtaatgaaactggaaaatgaactggagatggctcagcagtctgcaggtggacgagatactcggtttttacgtaatgaaatttgccaacttgaaaaacaattagaacaaaaagatagagaattggaggacatggaaaaggagttggagaaagagaagaaagttaatgagcaattggctcttcgaaatgaggaggcagaaaatgaaaacagcaaattaagaagagagaacaaacgtctaaagaaaaagaatgaacaactttgtcaggatattattgactaccagaaacaaatagattcacagaaagaaacacttttatcaagaagaggggaagacagtgactaccgatcacagttgtctaaaaaaaactatgagcttatccaatatcttgatgaaattcagactttaacagaagctaatgagaaaattgaagttcagaatcaagaaatgagaaaaaatttagaagagtctgtacaggaaatggagaagatgactgatgaatataatagaatgaaagctattgtgcatcagacagataatgtaatagatcagttaaaaaaagaaaacgatcattatcaacttcaagtgcaggagcttacagatcttctgaaatcaaaaaatgaagaagatgatccaattatggtagctgtcaatgcaaaagtagaagaatggaagctaattttgtcttctaaagatgatgaaattattgagtatcagcaaatgttacataacctaagggagaaacttaagaatgctcagcttgatgctgataaaagtaatgttatggctctacagcagggtatacaggaacgagacagtcaaattaagatgctcaccgaacaagtagaacaatatacaaaagaaatggaaaagaatacttgtattattgaagatttgaaaaatgagctccaaagaaacaaaggtgcttcaaccctttctcaacagactcatatgaaaattcagtcaacgttagacattttaaaagagaaaactaaagaggctgagagaacagctgaactggctgaggctgatgctagggaaaaggataaagaattagttgaggctctgaagaggttaaaagattatgaatcgggagtatatggtttagaagatgctgtcgttgaaataaagaattgtaaaaaccaaattaaaataagagatcgagagattgaaatattaacaaaggaaatcaataaacttgaattgaagatcagtgatttccttgatgaaaatgaggcacttagagagcgtgtgggccttgaaccaaagacaatgattgatttaactgaatttagaaatagcaaacacttaaaacagcagcagtacagagctgaaaaccagattcttttgaaagagattgaaagtctagaggaagaacgacttgatctgaaaaaaaaaattcgtcaaatggctcaagaaagaggaaaaagaagtgcaacttcaggattaaccactgaggacctgaacctaactgaaaacatttctcaaggagatagaataagtgaaagaaaattggatttattgagcctcaaaaatatgagtgaagcacaatcaaagaatgaatttctttcaagagaactaattgaaaaagaaagagatttagaaaggagtaggacagtgatagccaaatttcagaataaattaaaagaattagttgaagaaaataagcaacttgaagaaggtatgaaagaaatattgcaagcaattaaggaaatgcagaaagatcctgatgttaaaggaggagaaacatctctaattatccctagccttgaaagactagttaatgctatagaatcaaagaatgcagaaggaatctttgatgcgagtctgcatttgaaagcccaagttgatcagcttaccggaagaaatgaagaattaagacaggagctcagggaatctcggaaagaggctataaattattcacagcagttggcaaaagctaatttaaagatagaccatcttgaaaaagaaactagtcttttacgacaatcagaaggatcaaatgttgtttttaaaggaattgacttacctgatgggatagcaccatctagtgccagtatcattaattctcagaatgaatatttaatacatttgttacaggaactagaaaataaagaaaaaaagttaaagaatttagaagattctcttgaagattacaacagaaaatttgctgtaattcgtcatcaacaaagtttgttgtataaagaatacctaagtgaaaaggagacctggaaaacagaatctaaaacaataaaagaggaaaagagaaaacttgaggatcaagtccaacaagatgctataaaagtaaaagaatataataatttgctcaatgctcttcagatggattcggatgaaatgaaaaaaatacttgcagaaaatagtaggaaaattactgttttgcaagtgaatgaaaaatcacttataaggcaatatacaaccttagtagaattggagcgacaacttagaaaagaaaatgagaagcaaaagaatgaattgttgtcaatggaggctgaagtttgtgaaaaaattgggtgtttgcaaagatttaaggaaatggccattttcaagattgcagctctccaaaaagttgtagataatagtgtttctttgtctgaactagaactggctaataaacagtacaatgaactgactgctaagtacagggacatcttgcaaaaagataatatgcttgttcaaagaacaagtaacttggaacacctggagtgtgaaaacatctccttaaaagaacaagtggagtctataaataaagaactggagattaccaaggaaaaacttcacactattgaacaagcctgggaacaggaaactaaattaggtaatgaatctagcatggataaggcaaagaaatcaataaccaacagtgacattgtttccatttcaaaaaaaataactatgctggaaatgaaggaattaaatgaaaggcagcgggctgaacattgtcaaaaaatgtatgaacacttacggacttcgttaaagcaaatggaggaacgtaattttgaattggaaaccaaatttgctgagcttaccaaaatcaatttggatgcacagaaggtggaacagatgttaagagatgaattagctgatagtgtgagcaaggcagtaagtgatgctgataggcaacggattctagaattagagaagaatgaaatggaactaaaagttgaagtgtcaaaactgagagagatttctgatattgccagaagacaagttgaaattttgaatgcacaacaacaatctagggacaaggaagtagagtccctcagaatgcaactgctagactatcaggcacagtctgatgaaaagtcgctcattgccaagttgcaccaacataatgtctctcttcaactgagtgaggctactgctcttggtaagttggagtcaattacatctaaactgcagaagatggaggcctacaacttgcgcttagagcagaaacttgatgaaaaagaacaggctctctattatgctcgtttggagggaagaaacagagcaaaacatctgcgccaaacaattcagtctctacgacgacagtttagtggagctttacccttggcacaacaggaaaagttctccaaaacaatgattcaactacaaaatgacaaacttaagataatgcaagaaatgaaaaattctcaacaagaacatagaaatatggagaacaaaacattggagatggaattaaaattaaagggcctggaagagttaataagcactttaaaggataccaaaggagcccaaaaggtaatcaactggcatatgaaaatagaagaacttcgtcttcaagaacttaaactaaatcgggaattagtcaaggataaagaagaaataaaatatttgaataacataatttctgaatatgaacgtacaatcagcagtcttgaagaagaaattgtgcaacagaacaagtttcatgaagaaagacaaatggcctgggatcaaagagaagttgacctggaacgccaactagacatttttgaccgtcagcaaaatgaaatactaaatgcggcacaaaagtttgaagaagctacaggatcaatccctgaccctagtttgccccttccaaatcaacttgagatcgctctaaggaaaattaaggagaacattcgaataattctagaaacacgggcaacttgcaaatcactagaagagaaactaaaagagaaagaatctgctttaaggttagcagaacaaaatatactgtcaagagacaaagtaatcaatgaactgaggcttcgattgcctgccactgcagaaagagaaaagctcatagctgagctaggcagaaaagagatggaaccaaaatctcaccacacattgaaaattgctcatcaaaccattgcaaacatgcaagcaaggttaaatcaaaaagaagaagtattaaagaagtatcaacgtcttctagaaaaagccagagaggagcaaagagaaattgtgaagaaacatgaggaagaccttcatattcttcatcacagattagaactacaggctgatagttcactaaataaattcaaacaaacggcttgggatttaatgaaacagtctcccactccagttcctaccaacaagcattttattcgtctggctgagatggaacagacagtagcagaacaagatgactctctttcctcactcttggtcaaactaaagaaagtatcacaagatttggagagacaaagagaaatcactgaattaaaagtaaaagaatttgaaaatatcaaattacagcttcaagaaaaccatgaagatgaagtgaaaaaagtaaaagcggaagtagaggatttaaagtatcttctggaccagtcacaaaaggagtcacagtgtttaaaatctgaacttcaggctcaaaaagaagcaaattcaagagctccaacaactacaatgagaaatctagtagaacggctaaagagccaattagccttgaaggagaaacaacagaaagcacttagtcgggcacttttagaactccgggcagaaatgacagcagctgctgaagaacgtattatttctgcaacttctcaaaaagaggcccatctcaatgttcaacaaatcgttgatcgacatactagagagctaaagacacaagttgaagatttaaatgaaaatcttttaaaattgaaagaagcacttaaaacaagtaaaaacagagaaaactcactaactgataatttgaatgacttaaataatgaactgcaaaagaaacaaaaagcctataataaaatacttagagagaaagaggaaattgatcaagagaatgatgaactgaaaaggcaaattaaaagactaaccagtggattacagggcaaacccctgacagataataaacaaagtctaattgaagaactccaaaggaaagttaaaaaactagagaaccaattagagggaaaggtggaggaagtagacctaaaacctatgaaagaaaagaatgctaaagaagaattaattaggtgggaagaaggtaaaaagtggcaagccaaaatagaaggaattcgaaacaagttaaaagagaaagagggggaagtctttactttaacaaagcagttgaatactttgaaggatctttttgccaaagccgataaagagaaacttactttgcagaggaaactaaaaacaactggcatgactgttgatcaggttttgggaatacgagctttggagtcagaaaaagaattggaagaattaaaaaagagaaatcttgacttagaaaatgatatattgtatatgagggcccaccaagctcttcctcgagattctgttgtagaagatttacatttacaaaatagatacctccaagaaaaacttcatgctttagaaaaacagttttcaaaggatacatattctaagccttcaatttcaggaatagagtcagatgatcattgtcagagagaacaggagcttcagaaggaaaacttgaagttgtcatctgaaaatattgaactgaaatttcagcttgaacaagcaaataaagatttgccaagattaaagaatcaagtcagagatttgaaggaaatgtgtgaatttcttaagaaagaaaaagcagaagttcagcggaaacttggccatgttagagggtctggtagaagtggaaagacaatcccagaactggaaaaaaccattggtttaatgaaaaaagtagttgaaaaagtccagagagaaaatgaacagttgaaaaaagcatcaggaatattgactagtgaaaaaatggctaatattgagcaggaaaatgaaaaattgaaggctgaattagaaaaacttaaagctcatcttgggcatcagttgagcatgcactatgaatccaagaccaaaggcacagaaaaaattattgctgaaaatgaaaggcttcgtaaagaacttaaaaaagaaactgatgctgcagagaaattacggatagcaaagaataatttagagatattaaatgagaagatgacagttcaactagaagagactggtaagagattgcagtttgcagaaagcagaggtccacagcttgaaggtgctgacagtaagagctggaaatccattgtggttacaagaatgtatgaaaccaagttaaaagaattggaaactgatattgccaaaaaaaatcaaagcattactgaccttaaacagcttgtaaaagaagcaacagagagagaacaaaaagttaacaaatacaatgaagaccttgaacaacagattaagattcttaaacatgttcctgaaggtgctgagacagagcaaggccttaaacgggagcttcaagttcttagattagctaatcatcagctggataaagagaaagcagaattaatccatcagatagaagctaacaaggaccaaagtggagctgaaagcaccatacctgatgctgatcaactaaaggaaaaaataaaagatctagagacacagctcaaaatgtcagatctagaaaagcagcatttgaaggaggaaataaagaagctgaaaaaagaactggaaaattttgatccttcattttttgaagaaattgaagatcttaagtataattacaaggaagaagtgaagaagaatattctcttagaagagaaggtaaaaaaactttcagaacaattgggagttgaattaactagccctgttgctgcttctgaagagtttgaagatgaagaagaaagtcctgttaatttccccatttactaa

SEQ ID NO: 40 - DNA, 699 bp, unidentified
  source 1..699: mol_type = unassigned DNA
atgccaagccaacagaagaaaatcattttttgcatggccggagtgttcagttttgcatgtgccctcggagttgtgacagccttggggacaccgttgtggatcaaagccactgtcctctgcaaaacgggagctctgctcgtcaatgcctcagggcaggagctggacaagtttatgggtgaaatgcagtacgggcttttccacggagagggtgtgaggcagtgtgggttgggagcaaggccctttcggttctcattttttccagatttgctcaaagcaatcccagtgagcatccacgtcaatgtcattctcttctctgccatccttattgtgttaaccatggtggggacagccttcttcatgtacaatgcttttggaaaaccttttgaaactctgcatggtcccctagggctgtaccttttgagcttcatttcaggctcctgtggctgtcttgtcatgatattgtttgcctctgaagtgaaaatccatcacctctcagaaaaaattgcaaattataaagaagggacttatgtctacaaaacgcaaagtgaaaaatataccacctcattctgggtcattttcttttgcttttttgttcattttctgaatgggctcctaatacgacttgctggatttcagttcccttttgcaaaatctaaagacgcagaaacaactaatgtagctgcagatctaatgtactga

SEQ ID NO: 41 - DNA, 4389 bp, unidentified
  source 1..4389: mol_type = unassigned DNA
atggccctctattatgaccaccagatagaagccccggatgcagcagggtcaccctcatttatcagctggcaccctgtccatccattcttggcagttgcttacatcagcacaacctcaacaggcagcgtggatatttacctggagcaaggggagtgcgtgccagatacacacgtcgagaggccgttccgggttgcttccctgtgctggcacccgacgcggctggtgctggctgtgggctgggagactggagaagtgacggtgtttaacaagcaggacaaggagcagcacacgatgcccctgacacacacagccgacatcaccgtgctccgttggagccccagtggaaactgcctgctgtctggggacaggcttggtgtcttgctcttgtggaggttggaccaaaggggccgagtgcaagggacgcctctgctgaaacacgagtatgggaaacacctcacgcactgcatcttccggctcccccctcctggcgaggacctggttcagttggcaaaggcagctgtgagcggtgatgagaaagccctggacatgtttaactggaagaagagcagttctggaagtttgctgaagatggggtctcacgaggggctgttgttctttgtcagtctgatggacgggacagtgcactatgtggatgagaagggcaagaccactcaggtggtgtccgcagacagcacgattcagatgctgttctacatggagaagagggaggcactggtggtggtcacagagaacctccggctgtccctgtacacggtgcctcctgagggcaaagcagaagaagtgatgaaggtcaagctgagcgggaaaaccggccgccgggcagacatcgctttgattgaaggcagccttctcgtgatggccgtcggggaggctgccctcagattctgggacatagaacgaggagagaattatatactgagtccagatgagaagtttggctttgagaaaggagagaatatgaactgtgtgtgttactgtaaagtcaaaggtcttctggccgctggtaccgacagagggcgagtagccatgtggaggaaagtaccagacttcctgggcagccccggggcagagggcaaggacaggtgggcccttcagacccctaccgagctccaaggaaacatcacgcaaatccagtggggttccaggaagaacctgctggcagtgaacagcgtcatctccgtggccatcctcagcgagcgggccatgtcgtcacacttccaccagcaagtggccgccatgcaggtctccccgagtctgctgaatgtgtgcttcctgtccacgggggtcgcacacagcctgcgcaccgacatgcacatcagtggagtgtttgccaccaaggatgctgtcgcagtctggaacggaaggcaggtggcgatcttcgagctttctggagccgcgatacggagtgcagggaccttcttgtgtgagacgcctgtgttagcaatgcatgaagaaaacgtttacacggtggagtcaaaccgagttcaagttcgaacctggcaggggactgtcaaacaactcctccttttctcggagactgaggggaatccctgcttcttggacatctgtgggaatttcctggttgtagggacagacttggctcactttaaaagctttgatctttcccgaagagaggccaaagcacactgtagctgcaggagcctggcggagctggtccctggggtggggggcatcgcttctctgcggtgcagcagcagcgggagcaccatcagcatcctccccagcaaggctgacaacagccctgattccaaaatctgcttctacgatgttgaaatggacacagtgaccgtctttgacttcaagactggacaaattgatcggagagagacgctgtcctttaatgagcaagagactaataagagccacctctttgtggatgagggactgaaaaattatgttcccgtgaaccacttctgggaccagagtgagccccggctgtttgtatgcgaagccgtgcaggagacgccgcgctcccagcctcagtctgcaaacgggcagccccaagatgggcgcgctggccctgcggcagatgttttgatcctgtccttcttcatttccgaagagcacggcttcctgcttcatgagagcttcccccggcctgccacctcccacagtctcctggggatggaagtgccttattactacttcacaagaaagcccgaagaagcagacagagaagacgaggtggagcctgggtgccaccacatccctcagatggtgtccaggagacccctgcgagactttgtggggctggaggactgcgacaaggccacccgggacgccatgctccacttcagcttctttgtcaccataggagacatggacgaagccttcaaatccatcaagctcatcaaaagtgaggccgtctgggagaacatggcgcgcatgtgcgtgaagacccagcggctggacgtggccaaggtgtgcctggggaacatgggccatgcccgcggggcccgagcgctgcgtgaggcggagcaggagccggagctagaggcccgcgtggccgtgctggccacgcagctgggcatgctggaggacgccgagcagctgtacaggaagtgcaagcgccacgacctcctgaacaagttctaccaggctgcgggccggtggcaggaggccctccaggtagccgagcaccacgatcgcgtgcacctgcgcagcacctaccaccgctatgccgggcacctggaggccagcgccgactgcagccgggccctcagttactacgagaagtcggacacgcaccgcttcgaggtgcccaggatgctgtcggaggacctgccgtccctggagctctacgtgaataaaatgaaggataagaccctgtggcggtggtgggcgcagtacctggagagccagggcgagatggacgccgcgctgcactactacgagctggcccgggaccacttctccctggtccgcatccactgcttccagggcaatgtccagaaggctgcgcaaatagccaacgagacaggaaacctggcggcctcctaccacctcgcccgccagtacgagagccaggaggaggtcgggcaggcggtgcacttctacacccgggcacaggccttcaagaatgccatccgcctgtgcaaggagaacggcctggacgaccagctcatgaacttggccctgctgagctcccccgaggacatgatcgaggcggcccgatactacgaggagaagggcgtgcagatggacagggcggtcatgctgtaccacaaggctggccacttctccaaggccctggagctggcctttgccacccagcagtttgtggccctacagctcatagcagaggacctggatgagacgtcagaccctgcgctcctggcccgctgctccgacttcttcatcgagcacagtcagtacgagagggcggtagagctgctgctggctgccaggaagtatcaggaagccctgcagctgtgcctggggcagaacatgagcatcaccgaggagatggcggaaaagatgaccgtggccaaggactcctcggacctgcctgaggagtcgcggcgggagctgctggagcagatagcagactgctgcatgcgccagggcagctaccacctggccaccaagaagtacacgcaggccggcaacaagctgaaggccatgagggcgctgctcaaatccggagacacggagaaaatcacgttcttcgcgagcgtgtccaggcagaaggaaatctacatcatggctgctaactacctgcagtccctggactggcggaaggagccggagatcatgaagaacatcatcggcttctacaccaaggggcgggccctggacctcctggctggcttttatgacgcttgtgcccaggtggagattgatgaataccagaactacgacaaagcccacggggcgctgaccgaggcctacaagtgcctggccaaggccaaggccaagagccccctggaccaggagaccaggctggcgcagctgcagagcaggatggcactggtgaagaggttcatccaggcccgcaggacgtacacagaggaccccaaggagtccatcaagcagtgtgagctgctcctggaggaaccagacctggacagcaccatccgcatcggggacgtctatggcttcctggtggagcactacgtgcggaaggaggaataccagacggcctacagattcctggaggagatgcggcggcggcttcccttggccaacatgtcctactacgtgagcccgcaggccgtggacgccgtgcaccgggggctgggtctcccactgccacgcaccgtccccgagcaggtccgccacaacagcatggaggacgccagggagctggacgaggaggtggtggaagaggcagatgacgacccctga

SEQ ID NO: 42 - DNA, 5250 bp, unidentified
  source 1..5250: mol_type = unassigned DNA
atgcacttgaagcacctgaggaccctgctgagccctcaggatggagctgcaaaggtgacctgcatggcttggtcccagaacaatgccaaatttgctgtctgcacagtggaccgagtggtcttgctgtatgatgaacatggagaacggagagataaattctccaccaaaccagctgacatgaagtatggcaggaagagctatatggtgaagggcatggctttttctcctgattccactaaaattgccataggacagactgacaacatcatctatgtctacaagattggagaagattggggtgacaagaaagtcatctgcaacaagttcatccagacgagtgctgtcacttgtctgcaatggccggcagaatacatcattgtctttggactggctgaagggaaggttcgtttagcaaacaccaaaactaataaatcatctaccatctatgggacagagtcttacgtggtgtccctgacaacaaattgctctgggaaaggaattctctctggtcatgcagatggtaccatcgttaggtatttctttgatgatgaaggctctggagagtcacaggggaagttggttaaccacccgtgtccaccctatgccttggcatgggcaaccaatagcatcgtggctgcaggctgtgatcggaaaattgtagcctatggaaaagaaggtcacatgctacaaacttttgattatagccgtgaccctcaggagcgggagttcaccacagctgtatcaagtcctgggggccagtctgttgtgctaggaagttatgacaggcttcgggtgttcaactggatccctcgaagaagcatctgggaagaggcaaagcccaaggagattaccaatttatacaccatcactgccttggcctggaagcgggatggctcacggctctgtgtgggcacactatgtggtggggtggaacagtttgactgctgcctccgaaggagtatttacaagaacaagtttgagttgacgtatgtgggacctagccaggtgattgtgaagaacctgtcatcaggaacccgagtggtgctcaagtcacactatggctatgaggtggaagaggtgaaaatcctaggaaaggaacgttacttggtggctcacacatcagaaacactgctgctgggggacctgaacactaatcggcttagtgagatagcctggcaaggatctggtggcaatgagaagtatttctttgaaaatgagaatgtatgcatgatcttcaatgccggagagctaaccctggtggaatatgggaataatgacaccctgggttctgtacgcactgaattcatgaacccccacctcatcagtgttcgtattaatgagaggtgtcagcgaggaacagaagataataagaaattggcttatcttattgatattaagactattgctatagtggatctgattggtggctacaacattggcaccgtcagccatgagagccgtgtggattggctggaacttaatgagactggacacaagctcctcttcagggaccggaaacttcgtttgcatctgtatgatattgaaagctgctctaagacaatgatcctcaacttctgctcctatatgcagtgggtcccaggaagtgacgtgctggtagctcagaaccgaaacagtctgtgtgtatggtacaacattgaggcacctgagagagtcaccatgttcactattaggggtgatgttataggtctggagcggggcgggggaaagaccgaggtgatggtgatggaaggtgtgactactgttgcctacacattggatgagggcctcatcgagtttggaacagccattgatgatggcaactacatccgggcaacagccttcttagagactctggaaatgaccccagaaacagaggcaatgtggaaaaccttgagtaaactggcactagaggcaaggcaactacacattgcggagaggtgcttttctgctttgggccaagtagcaaaagctcgattcctgcatgagaccaatgagattgcagatcaagtatcccgggaatatggcggagaaggaacagacttttatcaggtccgagcacgtctagccatgctggaaaagaactacaaactggctgaaatgatctttttggaacagaatgctgtggaggaggccatgggcatgtaccaggagctacaccgttgggatgagtgtatcgctgtggctgaagccaaggggcacccagccctggagaagctacgtcgtagttactaccagtggctgatggacacacagcaagaggagcgagcaggtgaactacaggagagccaaggggatgggctagcagccatcagcctctacctcaaagctgggctccctgccaaagctgctcggctggtgctgacccgagaggaactgctagccaacacagagctggtagaacacatcactgcagcccttatcaagggggaactctacgaaagggcaggtgatctctttgagaagattcacaatccacagaaggccctggagtgctaccgtaaaggcaacgcattcatgaaagcggtagagctggctcgattggccttcccagtggaggtggtgaaactagaggaggcatggggggaccacctggtgcagcagaagcagcttgatgcagccattaatcactacatcgaagccaggtgctccattaaggcaattgaggccgccctgggtgcccgccagtggaagaaggcaatttatatattagatctacaggaccggaacactgcatccaaatactatcctctcgtggcccaacactatgcatccctgcaggagtatgagattgctgaggagctctatactaagggagatcggacaaaagatgccatagacatgtacacccaggctggtcgttgggaacaagcccacaagctggcgatgaaatgcatgagaccagaagatgtgtcagtgctatacatcactcaggcccaggaaatggagaagcagggcaagtaccgtgaggctgaaaggctatatgtgacagtacaagagcctgatcttgccatcaccatgtacaaaaagcacaagttgtatgatgacatgatccgcctggtagggaagcaccatccagatctcctcagtgatacacacctacatctgggcaaggagctggaggctgaaggccgactacaggaggctgagtaccactacctcgaggcccaggaatggaaggcaacagtgaacatgtaccgggccagtgggctttgggaagaggcctacagggtggccagaactcaaggaggggctaatgcccacaaacacgtggcctatctgtgggcaaagagcctgggaggagaggctgcagttagactgcttaataagctgggactcctggaagctgctgttgaccacgctgcagacaattgctcctttgaatttgcgtttgaactctctcggctggccctcaagcacaaaacccccgaggttcatctcaaatatgctatgttcctggaggatgagggtaaattcgaagaggctgaagctgaattcatcagagctggtaaacccaaggaggcagtcctcatgtttgtccataaccaggattgggaggcagctcagcgtgtggctgaggctcacgaccctgacagtgtcgccgaggtgcttgtgggacaggcccggggggccttggaggagaaggactttcagaaagcagaagggctgctgctccgggcccagagaccaggcctggccctcaattattataaggaggctggattatggagtgacgctctgcgcatctgcaaggactatgtgcccagccagctggaggctctgcaggaagaatatgagcgggaagctactaagaagggggccaggggtgtggagggatttgtggaacaagctcgacactgggagcaggctggagagtacagccgtgccgtggactgctacctcaaagtgcgagactctggaaacagcggcctggcggagaagtgctggatgaaggcagctgaactctccatcaagtttctgcctccccaacgtaatatggaagtcgttctggctgtaggaccccagctgattggaattggaaagcacagtgcagctgcagagctctatctgaatctggaccttgtcaaggaagcaatcgatgctttcatcgagggtgaggagtggaacaaggcgaagcgtgtagctaaggagttagatcccaggtatgaagactatgtggaccagcattataaagagttcctcaagaatcagggcaaagtggactcgctggtgggtgtggatgtgatagctgctttggacctgtatgtggagcagggccagtgggacaagtgcattgaaacagctaccaagcagaactacaagattctgcacaagtatgtggctttgtatgcaactcacttgatccgggagggtagctctgcccaggcattggccctgtatgtacagcacggagcccctgctaacccacagaacttcaatatctacaaaaggatcttcactgacatggtgagctctcctggaaccaactgtgccgaggcctatcatagctgggctgatcttcgagatgtcctcttcaacctgtgtgaaaacctggtgaagtccagtgaggcaaactctccagcccatgaggagttcaagacgatgctgctgatcgctcattactatgccacgcgctctgcagcccagagtgtcaaacagctggaaaccgtggctgccaggctttctgtttcactcttgcgtcacacccagctactacctgtagacaaagccttctatgaagcaggcattgctgccaaggcagttggctgggataacatggcattcatcttcctcaatcgctttttggacctgaccgatgcaatcgaggaagggactctagatggccttgaccactctgattttcaggatacagacattccctttgaggtgccactcccagctaagcagcatgtaccggaggctgagagagaagaggttcgagactgggtgcttacagtctccatggaccagcggctggagcaggttctgcctcgggatgagcgtggcgcctacgaggcctccctagtggcagcgagcactggtgttcgagccctgccctgccttattacaggataccccattctgaggaacaaaattgaatttaagcggccagggaaggctgctaacaaggacaactggaataaattccttatggccatcaagacctcccacagcccagtgtgccaggacgtgctgaaattcatcagtcagtggtgtggagggctccccagcaccagcttttcctttcagtag

SEQ ID NO: 43 - DNA, 1797 bp, unidentified
  source 1..1797: mol_type = unassigned DNA
atgaagccaacaggtacagacccaaggatcttatctatagctgctgaagttgcaaaaagccctgagcagaatgtccctgttatactgttgaagttaaaagaaataataaacatcacacctttaggaagctcagagttgaagaaaatcaaacaagatatatattgttatgatctcattcaatattgcctcttggtcctcagtcaagattattctcgaatccagggtggttggactacaatttcccagcttacacagatattaagccattgctgtgtgggcttggagccaggagaagatgcagaggaattttacaatgaattacttccatcagctgcagaaaattttctagttttggggagacaattacaaacatgttttatcaatgcagctaaggctgaagaaaaagatgaattactacactttttccaaattgtgactgattctctcttctggcttttgggaggccatgttgaacttattcagaatgtactacaaagtgatcatttcttacatttactgcaagctgacaatgtccaaataggatctgcagtcatgatgatgctacagaatatactacagatcaacagtggtgatttactcagaataggaagaaaagccctgtattcaattttagatgaagttattttcaagcttttttcaactcctagtccagttataagaagtactgctacaaaactcctactgttgatggctgaatcccatcaggaaattttgattttactgagacaaagtacctgctacaaaggactcagacgtctactaagtaaacaggaaactgggactgaattcagtcaagaacttagacagcttgttggccttttaagcccaatggtctatcaggaagtagaagagcagaaactacatcaagcagcatgcttgattcaagcctattggaagggttttcagacaagaaagagattaaagaagcttccatctgctgtgattgctttgcagaggagtttcagatccaaacgatcaaagatgttgctggagataaataggcagaaggaagaagaggacctcaaattacaattgcaacttcaaagacagagagccatgagactttcccgagaattgcagctgagtatgctcgaaatagttcatccaggtcaggtggagaaacactatcgggaaatggaagagaaatcagcactgattatccagaaacattggagagggtacagggaaaggaaaaattttcaccaacagaggcagtctctcatagagtataaagcagctgtcacacttcaaagagcagcgcttaaattcctagcgaagtgccgtaagaaaaagaaactatttgctccttggcgaggactccaagaactcactgatgcacgccgagttgaactgaagaaacgagtggatgactatgtcagaagacatttgggctctccaatgtcagatgtggtcagtagggagctccatgcccaagctcaagaacgactgcaacactactttatgggcagggccctagaagagcgagcccagcagcacagagaagctctgatagcacagatcagcaccaacgttgaacagctaatgaaggcaccaagtctgaaggaggcagaagggaaagaacctgagctcttcctaagtagatccaggcctgtggcagccaaggccaagcaggcccatctcacaaccctgaagcacatacaagcaccctggtggaagaagcttggagaagaatctggagatgagattgatgttccaaaggatgagcttagtatagaattagaaaatttattcattggtggaaccaaaccaccttag

SEQ ID NO: 44 - DNA, 2022 bp, unidentified
  source 1..2022: mol_type = unassigned DNA
atgagccggaccctcgcatcggccgtgcccctgtcgagtcccgactactacgagaggctgggccaactccagcacgggctgcgggacagtgaaaagaagagattggacctggaaaagaaactttatgaatataatcagtctgatacatgcagagttaagctgaaatatgtaaaactaaagaattatctgaaggaaatatgtgaatctgaaaagaaggctcatactcgaaaccaagaatatttaaagcgatttgagcgtgtccaagctcatgttgtacacttcaccacaaatacagagaagcttcaaaaactgaagctcgaatatgagactcaaattaagaagatgctatgctcaaaagatagcctgggactaaaagaggaactgacagatgaagacagagaaaaggttgcagtgcacgaggggattaactcaggaacagccatgtcaagaggattgtatcaaccagcaacaatctttatgggccgccaaatgtcagccatcttaagcatgagagatttcagtacagagcacaaatctccccagcccacaaagaacttttcaattcctgacccacattcacaccgacagacagcccagagcagtaatgtgacagacagctgtgtagtacaaactagtaatgacacacagtgcttaaataagtctgacaacatagatggaaaggcatctcttcagattggtgagaaaatgccagtcacagccagtgtattgtctgaggaggaacaaactcattgcttggagataggaagtaacacacgtcatggcaagagtaatttatctgaaggcaaaaagtctgctgaactcaattccccgttacgggaaagattaagtccagagaacagaaccactgatttaaagtgtgacagttccagcggatcagagggagaaatactgacacgggaacatattgaagttgaggaaaaaagagccagcccgccagtctctccgataccagtttcagaatactgtgaatctgaaaataagtggtctcaagagaagcattctccttgggaaggtgtttcagatcatcttgctcacagggaaccaaagtcacaaaagcccttcagaaaaatgcaggaagaggaggaggaaagttggagcaccagcagtgaccttaccatttcaataagtgaagatgatctgattttagagagcccagaaccacagccaaatccaggtggcaagatggagggagaagatggaatagaggccttaaaattaatccatgctgagcaagaaagagttgccctatccactgaaaaaaattgtattttgcaaaccctaagctctcctgattcagaaaaggaatcctccactaacgcaccaacaagagaacctggacaaacaccagactcagacgtaccgagggcacaggtgggtcagcatgttgccaccttgaaagaacatgataattctgtcaaagaagaggcaacagcattattgagaaaagcccttacagaagagtgtggccgtaggtcagctattcacagtagtgaatcatcttgcagcttgccatctattctgaatgacaatagtggaataaaggaagccaaacctgctgtatggctcaacagtgttcctacaagggaacaagaagtttcaagtggctgtggagacaagagcaagaaagaaaatgtggctgcagatatcccaatcacagaaacagaagcctatcagttgctgaagaaggccacccttcaggataatacaaatcaaactgaaaacaggtttcaaaagacagatgcttctgtgtcacacttgtcaggtttgaatattggcagcggtgcattcgagacaaagacagctaacaaaattgcttcggaagctagtttttcatctagtgaaggaagtcctttgtcaaggcatgaaaacaaaaagaaacccgtgatcaatttaaaatctaatgccctctgggatgagtctgatgacagtaactcagaaattgaggctgctttacgccccagaaaccataacaccgatgattctgatgatttttatgactaa

SEQ ID NO: 45 - DNA, 2094 bp, unidentified
  source 1..2094: mol_type = unassigned DNA
atgggggaaagagcaggaagtccaggtactgatcaagaaagaaaggcaggcaaacaccattattcttacttatctgattttgaaacgccacagtcttctggccgatcatcgctggtcagttcttcacctgcaagtgttaggagaaaaaatcctaaaagacaaacttcagatggccaagtacatcaccaagcccctcggaaaccaagccctaagggtctaccaaacagaaagggagtccgagtgggatttcgctcccagagcctcaatagagagccacttcggaaagatactgatcttgttacaaaacggattctgtctgcaagactgctaaaaatcaatgagttgcagaatgaagtatctgaactccaggtcaagttagctgagctgctaaaagaaaataaatctttgaaaaggcttcagtacagacaggagaaagccctgaataagtttgaagatgccgaaaatgaaatctcacaacttatatttcgtcataacaatgagattacagcactcaaagaacgcttaagaaaatctcaagagaaagaacgggcaactgagaaaagggtaaaagatacagaaagtgaactatttaggacaaaattttccttacagaaactgaaagagatctctgaagctagacacctacctgaacgagatgatttggcaaagaaactagtttcagcagagttaaagttagatgacaccgagagaagaattaaggagctatcgaaaaaccttgaactgagtactaacagtttccaacgacagttgcttgctgaaaggaaaagggcatatgaggctcatgatgaaaataaagttcttcaaaaggaggtacagcgactatatcacaaattaaaggaaaaggagagagaactggatataaaaaatatatattctaatcgtctgccaaagtcctctccaaataaagagaaagaacttgcattaagaaaaaatgctgcatgccagagtgattttgcagacctgtgtacaaaaggagtacaaaccatggaagacttcaagccagaagaatatcctttaactccagaaacaattatgtgttacgaaaacaaatgggaagaaccaggacatcttactttggacttgcaatctcaaaagcaagacaggcatggagaagcagggattctaaacccaattatggaaagagaagaaaaatttgttacagatgaagaactccatgtcgtaaaacaggaggttgaaaagctggaggatgaatgggaaagagaagaacttgataaaaagcaaaaagaaaaggcatctttactggaaagagaagaaaagccagagtgggaaactggaaggtaccaactaggaatgtatccaattcagaatatggataaattgcaaggagaggaagaagaaagactgaagagagaaatgctacttgctaaactgaatgaaattgacagagaactccaagattctcgaaatctaaaataccctgttttgccattgttacctgattttgaatcaaaactacactccccagagagaagccccaaaacatacaggttctctgaatcctcagagagattatttaatgggcatcatttgcaagacatcagtttctcaactccaaaaggagaaggtcagaattcaggaaatgttagaagtccagcctcccctaatgagttcgcatttggtagctacgtgccttcgtttgcaaaaacatcagagaggtcaaatccatttagtcaaaaaagtagttttttggatttccaaagaaacagtatggaaaaacttagtaaagatggtgtagatttaattacaagaaaagagaaaaaagctaatttgatggaacagttatttggtgccagtggtagcagcaccatttcctccaaaagcagtgacccaaattctgtggcttccagtaaaggagacattgaccctctaaattttctccctgggaataaaggcagcagagatcaagaacatgatgaagatgaaggctttttcctcagtgaaggaagaagttttaatccaaataggcaccgattaaaacatgcagacgataaaccagcagtaaaagcagctgattctgtagaagatgaaattgaagaagtagcactgagatga

SEQ ID NO: 46 - DNA, 1947 bp, unidentified
  source 1..1947: mol_type = unassigned DNA
atgaaccgatacacaaccatgagacagttgggggacggcacgtatgggagtgtgcttatgggcaagagtaatgaatccggggagctggtggccatcaaaaggatgaagagaaagttctattcttgggatgaatgcatgaacttgagagaagttaagtctctgaagaaacttaatcatgccaatgttattaaattgaaagaagttatcagagaaaatgaccatctttattttatatttgaatatatgaaagaaaacctctatcaattaatgaaagacagaaacaagttgttccctgaatcagtcatcagaaatattatgtatcaaatattgcaagggctggcttttatccataaacatggcttttttcatagggacatgaaaccagaaaacttgctttgtatgggtccagagcttgtgaaaattgctgattttggacttgcaagagaattaaggtcacagccaccatacactgattatgtatctaccagatggtatcgtgcccctgaagttttactgagatcttcagtttatagttctcccattgatgtgtgggctgttggaagtatcatggctgaactctatatgttaaggccacttttcccagggacaagtgaggtcgatgaaatctttaaaatttgccaagttttagggactcccaaaaaaagtgactggccagaaggataccagctggcatcctctatgaacttccgttttccccagtgtgttcctataaacttaaaaactcttattcccaatgccagtaatgaagctattcagctcatgaccgaaatgttgaattgggatccaaagaaacgaccgacagcaagccaggcattgaaacacccatattttcaagttggtcaggtattaggcccttcgtcaaatcatctggaatcaaaacagtctttaaataagcagctgcaaccattagaatcaaagccatctttagttgaggtagagcctaagcctctgccggatataatcgatcaggttgttggacaaccccagccaaaaactagccagcagccactgcagcccattcagccgccacagaacctgagcgtccagcaacctccaaagcaacagagtcaggagaaaccgccacaaacgctattcccgagcatcgtcaaaaacatgccaactaagccaaatggcacactgagtcataaaagtggtaggaggcgttggggtcagactatcttcaagtctggagatagctgggaagagttggaggactatgatttcggagcctcccattccaagaagccaagcatgggtgtttttaaagaaaaaaggaaaaaagattctccatttcggcttccagagccagtaccctcaggctccaaccactcgacaggggaaaacaagagcttacctgctgttacttccctaaaatctgattccgaattgtcaactgctccaacctctaaacagtactacttgaaacaatcaagatatcttccaggtgtgaatcccaagaaggtgtccttgatagccagtggaaaggaaataaacccccacacttggagcaaccagttattccccaagtcactgggacccgttggggcagaacttgctttcaaaaggagcaatgcagaagaaagcataattaaaccaatcgaaaaactatcatgcaatgaaacttttcctgaaaaattagaggacccacaaggaaatcttggaagttatgctacttacaatcagtcaggatatattccttcctttctcaaaaaagaagtgcagtcagctggccagaggatccacttagcacctctcaatgcaacggcttcagaatatacctggaacacaaaaactggtcgggggcagttttcaggacgtacttataatcctacagcaaaaaacctaaatattgtgaaccgtgcacagcccattccctcagtgcatgggaggacagactgggtggccaagtatggaggccaccggtag

SEQ ID NO: 47 - DNA, 1338 bp, unidentified
  source 1..1338: mol_type = unassigned DNA
atgccttcccgggctgaggactatgaagtgttgtacaccattggcacaggctcctacggccgctgccagaagatccggaggaagagtgatggcaagatattagtttggaaagaacttgactatggctccatgacagaagctgagaaacagatgcttgtttctgaagtgaatttgcttcgtgaactgaaacatccaaacatcgttcgttactatgatcggattattgaccggaccaatacaacactgtacattgtaatggaatattgtgaaggaggggatctggctagtgtaattacaaagggaaccaaggaaaggcaatacttagatgaagagtttgttcttcgagtgatgactcagttgactctggccctgaaggaatgccacagacgaagtgatggtggtcataccgtattgcatcgggatctgaaaccagccaatgttttcctggatggcaagcaaaacgtcaagcttggagactttgggctagctagaatattaaaccatgacacgagttttgcaaaaacatttgttggcacaccttattacatgtctcctgaacaaatgaatcgcatgtcctacaatgagaaatcagatatctggtcattgggctgcttgctgtatgagttatgtgcattaatgcctccatttacagcttttagccagaaagaactcgctgggaaaatcagagaaggcaaattcaggcgaattccataccgttactctgatgaattgaatgaaattattacgaggatgttaaacttaaaggattaccatcgaccttctgttgaagaaattcttgagaaccctttaatagcagatttggttgcagacgagcaaagaagaaatcttgagagaagagggcgacaattaggagagccagaaaaatcgcaggattccagccctgtattgagtgagctgaaactgaaggaaattcagttacaggagcgagagcgagctctcaaagcaagagaagaaagattggagcagaaagaacaggagctttgtgttcgtgagagactagcagaggacaaactggctagagcagaaaatctgttgaagaactacagcttgctaaaggaacggaagttcctgtctctggcaagtaatccagaacttcttaatcttccatcctcagtaattaagaagaaagttcatttcagtggggaaagtaaagagaacatcatgaggagtgagaattctgagagtcagctcacatctaagtccaagtgcaaggacctgaagaaaaggcttcacgctgcccagctgcgggctcaagccctgtcagatattgagaaaaattaccaactgaaaagcagacagatcctgggcatgcgctag

SEQ ID NO: 48 - DNA, 3039 bp, unidentified
  source 1..3039: mol_type = unassigned DNA
atgatggcgcagtccaacatgtttaccgtggctgatgtgttgagtcaagatgaactgcgcaaaaagctataccagacgtttaaggatcggggtatactggatacactcaagacacaacttcgaaaccagctaattcatgagttgatgcaccctgtattgagtggagaactgcagcctcggtccatttcagtagaagggagctccctcttaataggcgcctctaactctttagtggcagatcacttacaaagatgtggctatgaatattcactttctgttttctttccagaaagtggtttggcaaaagaaaaggtatttactatgcaggatctattacaactcattaaaatcaaccctacttccagtctctacaaatcactggtttcaggatctgataaagaaaatcaaaaaggttttcttatgcattttttaaaagaattggcagaatatcatcaagctaaagagagttgtaatatggaaactcagacaagttcgacatttaacagagattctctggctgagaagcttcagcttattgatgatcagtttgcagatgcttaccctcagcgtatcaagttcgaatctttagaaataaagctaaatgagtataagagagaaatagaagagcaacttcgggcagaaatgtgtcaaaagttgaagttttttaaagataccgagatagcaaaaattaaaatggaagcaaaaaaaaagtatgaaaaggagttaaccatgttccagaatgattttgaaaaagcttgtcaagcaaaatctgaagctctcgttcttcgggaaaagagtacccttgaaagaattcacaagcaccaagagattgaaacaaaagaaatttatgctcaaaggcaacttttactaaaagatatggatttgctaagaggaagagaagcagagctgaagcaaagagttgaagcttttgaattgaaccagaagctccaggaagaaaaacataaaagcataactgaggcacttaggagacaggagcagaatataaagagttttgaggagacctatgaccgaaagctcaagaatgaacttctaaagtatcaacttgaactgaaggatgactacatcattagaactaatcgactgattgaagatgaaaggaagaataaagaaaaagctgttcatttgcaagaggagctcatagctattaattcaaaaaaggaggaactcaatcaatctgtaaatcgtgtgaaagaacttgagcttgaattagagtctgtcaaagcccagtctttggcaataacaaaacaaaaccatatgctgaatgaaaaggttaaagagatgagtgattattcactactaaaagaagagaaactggagcttctggcacaaaataaattacttaaacaacaactggaagagagtagaaatgaaaacctgcgtctcctaaaccgcctagctcagccggctcctgaacttgcagtctttcagaaagaactacggaaagccgaaaaggctatagtggttgagcatgaggagttcgaaagctgcaggcaagctctgcacaaacaactgcaagacgaaattgagcattctgcacagctgaaggcccagattctaggttacaaagcttctgtaaagagtttaactactcaggttgccgatttaaaattgcaactgaagcaaactcagacagccctagagaatgaagtgtactgcaatccaaagcagtctgtgatcgatcgttctgtcaatggattaataaatggcaatgtggtgccttgcaatggtgagataagtggggatttcttgaacaatccttttaaacaggaaaacgttctagcacgtatggttgcatcaaggatcacaaattatccaactgcatgggtggagggtagttcccctgattctgaccttgagtttgtagccaatactaaggcaagggtcaaagagcttcagcaagaggccgaacgcttggaaaaggctttcagaagttaccatcggagagtcattaaaaactctgccaaaagcccactagcagcaaagagcccaccatctctgcacttgctggaagccttcaaaaacattacttccagttccccggaaagacatatttttggagaggacagagttgtctctgagcagcctcaagtgggcacacttgaagaaaggaatgacgtcgtggaagcactgacaggcagtgcagcctcgaggctccgcgggggcacttcctccagacgcctctcttccacaccccttccaaaagcaaaaagaagcctcgaaagtgaaatgtatctggaaggtctgggcagatcacacattgcttcccccagtccttgtcctgacagaatgcccctaccatcacccactgagtctaggcacagcctctccatccctcctgtctccagccctccggagcagaaagtgggtctttatcgaagacaaactgaacttcaagacaaaagtgaattttcagatgtggacaagctagcttttaaggataatgaggagtttgaatcatcttttgaatctgcagggaacatgccaaggcagttggaaatgggcgggctttctcctgccggggatatgtctcatgtggacgctgctgcagctgctgtgcccctctcatatcagcacccaagtgtagatcagaaacaaattgaagaacaaaaggaagaagaaaaaatacgggaacagcaagtgaaagaacgaaggcagagagaagaaagaaggcagagtaacctacaagaagttttagaaagggaacgaagagaactagaaaaactgtatcaggaaaggaagatgattgaagaatcactgaagattaaaataaaaaaggaattagaaatggaaaatgaattagaaatgagtaatcaagaaataaaagacaaatctgctcacagtgaaaatcctttagagaaatacatgaaaatcatccagcaggagcaagaccaggagtcggcagataagagctcaaaaaagatggtccaagaaggctccctagtggacacgctgcaatctagtgacaaagtcgaaagtttaacaggcttttctcatgaagaactagacgactcttggtaa

SEQ ID NO: 49 - DNA, 666 bp, unidentified
  source 1..666: mol_type = unassigned DNA
atgtcggactctgaggaggagagccaggaccggcaactgaaaatcgtcgtgctgggggacggcgcctccgggaagacctccttaactacgtgttttgctcaagaaacttttgggaaacagtacaaacaaactataggactggatttctttttgagaaggataacattgccaggaaacttgaatgttacccttcaaatttgggatataggagggcagacaataggaggcaaaatgttggataaatatatctatggagcacagggagtcctcttggtatatgatattacaaattatcaaagctttgagaatttagaagattggtatactgtggtgaagaaagtgagcgaggagtcagaaactcagccactggttgccttggtaggcaataaaattgatttggagcatatgcgaacaataaaacctgaaaaacacttacggttttgccaggaaaatggttttagtagccactttgtctcagccaagacaggagactctgtcttcctgtgctttcagaaagttgctgctgaaatccttgggatcaaattaaacaaagcagaaatagaacagtcacagagggtggtgaaggcagatattgtaaactacaaccaggaacctatgtcaaggactgttaaccctcctagaagctctatgtgtgcagttcagtga

SEQ ID NO: 50 - DNA, 6470 bp, unidentified
  source 1..6470: mol_type = unassigned DNA
tgagtgataccccttctactggtttttccatcattcatcctacgtcttctgaaggtcaagttccaccccctcgccatttgagcctcactcatcctgttgtggccaagcgaatcagtttctacaagagcggagacccccaattcggcggggtcagggtggtggtcaaccctcgctcctttaagtcctttgatgctctgctggataacttgtccaggaaggtgcccctcccttttggagtgaggaacatcagcacccctcggggcaggcacagcatcacgcgcctggaggagctggaggacggcgagtcctacctatgttcccacggcaggaaggtgcagcctgtagacctggacaaagcccgtcggcgcccgcggccctggctcagcagccgggccattagcgcgcactcaccgccccaccccgtagccgtcgctgctcccggcatgccccgccccccacggagcctagtggtcttcaggaatggcgacccgaagacgaggcgtgcggttcttctgagcaggagggtcacccagagcttcgaggcatttctacagcacctgacagaggtcatgcagcgccctgtggtcaagctgtacgctacggacggaaggagggttcccagcctccaggcagtgatcctgagctctggagctgtggtggcggcaggaagggagccatttaaaccaggaaattatgacatccaaaaatacttgcttcctgctagattaccagggatctctcagcgtgtgtaccccaagggaaatgcaaagtcagaaagcagaaagataagcacacatatgtcttcaagctcaaggtcccagatttattctgtttcttctgagaaaacacataataatgattgctacttagactattcttttgttcctgaaaagtacttggccttagaaaagaatgattctcagaatttaccaatatatccttctgaagatgatattgagaaatcaattatttttaatcaagacggcactatgacagttgagatgaaagttcgattcagaataaaagaggaagaaaccataaaatggacaactactgtcagtaaaactggtccttctaataatgatgaaaagagtgagatgagttttccaggaagaacagaaagtcgatcatctggtttaaagcttgcagcatgttcattctctgcagatgtgtcacctatggagcgaagcagtaatcaagagggcagtttggcagaggagataaacattcaaatgacagatcaagtggctgaaacttgcagttctgctagttgggagaatgctactgtggacacagatatcatccagggaactcaagaccaagcaaagcatcgtttttataggccccctacacctggactaagaagagtgagacaaaagaaatctgtgattggcagtgtgaccttagtatctgaaactgaggttcaagagaaaatgattggacagttttcatatagtgaagaaagggaaagtggggaaaacaagtctgagtatcacatgtttacacattcttgcagtaaaatgtcatcagtatctaacaaaccagtacttgttcagatcaataacaatgatcaaatggaggagtcatcattagaaagaaaaaaggaaaacagtctgcttaagtcaagtgcaataagtgctggtgttatagaaattacaagtcagaagatgttagagatgtcacataataatggtttgccatcaactatatcaaataactcaattgtggaggaagatgtagttgattgtgtggtattggacaacaaaactggtatcaagaacttcaaaacttatggtaacaccaatgataggttcagtcctatttcagcagatgcaacccatttttcaagtaataactctggaactgacaaaaatatttctgaggctccagcttcagaagcatcctctactgtcactgcaagaattgacagactaattaatgaatttgctcagtgtggtttaacaaaacttccaaaaaatgaaaagaagattttgtcatctgttgccagcaaaaagaagaaaaaatctcgacagcaagcaataaattccaggtatcaagatggacagcttgcaaccaaaggaattcttaataagaatgagagaataaacacaaaaggtagaattacaaaggaaatgatagtgcaagattcagatagtccccttaaaggagggatactttgtgaggaagacctccagaaaagtgatactgtaattgaatcaaatactttttgttccaaaagtaatctcaattccacgatttccaagaatttccatagaaataaattaaatactactcaaaattccaaggttcaaggacttttaaccaaaagaaaatctagatcactaaataaaataagcttaggagcacctaaaaaaagagaaatcggtcaaagagataaagtgtttcctcacaatgaatctaaatattgcaaaagtacttttgaaaacaaaagtttatttcatgtatttaacatccttgagcaaaaacccaaagatttttatgcaccgcaatctcaagcagaagtggcatctgggtatttgagaggaatggcaaagaagagtttagtttcaaaagttactgattcacacataactttaaaaagccagaaaaaacgtaaaggggataaagtgaaagcaagtgctattttaagtaaacaacatgctacaaccagggcaaattctttagcttctttgaaaaaacctgattttcctgaggctattgctcatcattcaattcaaaattatatacagagttggttgcagaacataaatccatatccaactttaaagcctataaaatcagctccagtatgtagaaatgaaacgagtgtggtaaattgtagcaataatagtttttcagggaatgatccccatacaaattctggaaaaataagtaattttgttatggaaagtaataagcacataactaaaattgccggtttgacaggagataatctatgtaaagagggagataagtcttttattgccaatgacactggtgaagaagatctccatgagacacaggttggatctctgaatgatgcttatttggttcccctgcatgaacactgtactttgtcacagtcagctattaatgatcataatactaaaagtcatatagctgctgaaaaatcaggaccagagaaaaaacttgtttaccaggaaataaacctagctagaaaaaggcaaagtgtagaggctgccattcaagtagatcctatagaagaggaaactccaaaagacctcttaccagtcctgatgcttcaccaattgcaagcttcagttcctggtattcacaagactcagaatggagttgttcaaatgccaggttcacttgcaggtgttccctttcattctgcaatatgtaattcatccactaatctccttctagcttggctcttggtgctaaacctaaagggaagtatgaatagcttctgtcaagttgatgctcacaaggctaccaacaaatcttcagaaacacttgcattgttggagattctaaagcacatagctatcacagaggaagctgatgacttgaaagctgctgttgccaatttagtggagtcaactacaagccactttggactcagtgagaaagaacaagacatggttccaatagatctttctgcaaattgttccacggtcaacattcagagtgttcctaagtgcagtgaaaatgaaagaacacaaggaatctcctctttggatggaggttgctctgccagtgaggcatgtgcccctgaagtctgtgttttggaagtgacttgctctccatgtgagatgtgcactgtaaataaggcttattctccaaaagagacatgtaaccccagtgacactttttttcctagtgatggttatggtgtggatcagacttctatgaataaggcttgtttcctaggagaggtctgttcacttactgatactgtgttttctgataaggcttgtgctcaaaaggagaaccatacctatgagggagcttgcccaattgatgagacctacgttcctgtcaatgtctgcaataccattgactttttaaactccaaagaaaacacatatactgataacttggattcaactgaagagttagaaagaggtgatgacattcagaaagatctaaatattttgacagaccctgaatataaaaatggatttaatacattggtgtcacatcaaaatgtcagtaatttaagctcctgtggcctttgcctaagtgaaaaagaagcagaacttgataagaaacatagttctctagatgattttgaaaattgttcactaaggaagtttcaggatgaaaatgcatatacttcctttgatatggaagaaccacggacttctgaagaaccaggctcaataaccaacagcatgacatcaagtgaaagaaacatttcagaattggaatcttttgaagaattagaaaaccatgacactgatatctttaatacagtggtaaatggaggagagcaagccactgaagaattaatccaagaagaggtagaggctagtaaaactttagaattgatagacatctctagtaagaatattatggaagaaaaaagaatgaacggtataatttatgaaataatcagtaagaggctggcaacaccaccatctttagatttttgctatgattctaagcaaaatagtgaaaaggagaccaatgaaggagaaactaagatggtaaaaatgatggtgaaaactatggaaactggaagttattcagagtcctctcctgatttaaaaaaatgcatcaaaagtccagtgacttctgattggtcagactatcggcctgacagtgacagtgagcagccatataaaacatccagtgatgatcccaatgacagtggcgaacttacccaagagaaagaatataacataggatttgttaaaagggcaatagaaaaactgtacggtaaagcagatattatcaaaccatctttttttcctgggtctacccgcaaatctcaggtttgtccttataattctgtggaatttcagtgttccaggaaagcaagtctttatgattctgaagggcagtcatttggctcttctgaacaggtatctagtagttcatctatgttgcaggaattccaggaggaaagacaagataagtgtgatgttagtgctgtgagggacaattattgtaggggtgacattgtagaacctggtacaaaacaaaatgatgatagcagaatcctcacagacatagaggaaggagtactgattgacaaaggcaaatggcttctgaaagaaaatcatttgctaaggatgtcatctgaaaatcctggcatgtgtggcaatgcagacaccacatcagtggacaccctacttgataataacagcagtgaggtaccatattcacattttggtaatttggccccaggcccaacgatggatgaactctcctcttcagaactcgaggaactgactcaaccccttgaactaaaatgcaattactttaacatgcctcatggtagtgactcagaaccttttcatgaggacttgctggatgttcgcaatgaaacctgtgccaaggaaagaatagcaaatcatcatacagaggagaagggtagtcatcagtcagaaagagtatgcacatctgtcactcattcctttatttctgctggtaacaaagtctaccctgtctctgatgatgctattaaaaaccaaccattgcctggcagtaatatgattcatggtacacttcaggaagctgactctttggataaactgtatgctctttgtggtcaacattgcccaatactaactgttattatccaacccatgaatgaggaagaccgaggatttgcatatcgcaaagaatctgatattgaaaatttcttgggtttttatttatggatgaaaatacacccatatttacttcagacagacaaaaatgtgttcagggaagagaacaataaagcaagtatgagacaaaatcttattgataatgccattggtgatatatttgatcagttttatttcagtaacacatttgacttgatgggtaaaagaagaaaacaaaaaagaattaacttcttggggttagaggaagaaggtaatttaaagaaatttcaaccagatttgaaggaaaggttttgtatgaatttcttgcacacatcattgttagttgtgggtaatgtggattcaaatacacaagacctcagcggtcagacaaatgaaatctttaaagcagtcgatgagaataacaacttattaaataacagattccagggctcaagaacaaatctcaaccaagtagtaagagaaaatatcaactgtcattacttctttgaaatgcttggtcaagcttgcctcttagatatttgccaagttgagacctccttaaatattagcaacagaaatattttagaactttgtatgtttgagggtgaaaatcttttcatttgggaagaggaagacatattaaatttaactgatcttgaaagcagtagagaacaagaagatttataa

SEQ ID NO: 51 - DNA, 7203 bp, unidentified
  source 1..7203: mol_type = unassigned DNA
atgaacagcacccccaggaatgcccaggccccgagccaccgtgagtgcttcctgccctctgtggctcgcaccccctcggtcaccaaggtcacgccagccaagaagatcaccttcctcaagcgaggggatccacggtttgctggggtccgcctggccgttcaccagcgcgcctttaagaccttcagcgccctcatggacgagctctcccagcgcgtgcctctctcctttggggtgcgctctgtcaccacaccccggggcctgcatagcctcagcgccctggagcagctggaagatggaggctgctacctctgctctgataagaagccccccaagacccccagtggaccaggccggccacaggagagaaaccccactgctcagcagttgcgggatgtcgaaggccagcgtgaagccccaggcacctcctcctcccggaagagtcttaaaaccccccggaggatactgctgattaagaacatggaccctcgcctccagcagacagtggttctcagtcacaggaatactaggaacctggccgcctttctcggcaaagcctcagatctcctgcgctttcctgtgaagcagttgtacacgaccagcgggaaaaaggtggactcgctgcaggccctgctgcacagcccctctgtgctggtgtgtgccgggcatgaggccttcagaaccccagccatgaaaaatgccaggagaagcgaggctgaaactttatctgggctgacttcaagaaacaaaaacgggagctgggggccaaagaccaagccgagtgtgatccattcgcggtctccgccaggcagcacgccacggctgccagaaaggcctggtcctagcaaccccccggtgggccctgctcctggcaggcaccctcaggacacgccagctcagtcgggcccgctggtggctggcgatgacatgaagaagaaggtccgcatgaatgaggacggcagcctgtccgtggagatgaaagtccgcttccacctggtcggcgaggacacgctcctatggtcccggaggatgggcagggccagcgccctcacggcagccagtggggaagaccccgttctgggggaggtagaccccctctgctgtgtgtgggagggctacccttggggcttctcagagcctggggtgtggggaccccggccctgcagggtgggatgcagggaagtctttggccgaggcgggcagccagggcccaagtatgaaatctggacgaatcccctgcatgcctcccagggagagagagtggcagctcggaagaggtggggactggcccagcacgtccgctgcagtggcctgtggggccacgggactgccgggagggagagatgcagccaggacagtgccagcccagcctccagcaccggcctccccgagggctcggagccagagtcctcctgctgccccaggaccccggaggacggggtggacagtgccagcccctctgcccagataggggctgagcggaaagctggagggagcctgggtgaggaccccggcctatgcatagatggagcagggctgggcggcccagagcaaggcggccgcctgacaccgagggcccggagtgaggagggggcttcttcggactcgtcagccagcaccggctctcatgagggatccagcgaatggggtgggcggccccagggctgtccaggcaaggcaagggccgagacctctcagcaggaggccagcgagggaggcgaccccgcttctccagccctgagtctttcatctttaaggagtgacgacctgcaggcagagacgcaaggacagggcaccgagcaggccacgggagcggctgtgacaagggagcctctggttctgggcctttcctgctcctgggactcggaaggagcctcttccaccccttccacctgcacttcatcccagcaggggcagagaaggcacagaagccgggccagtgcaatgtcctcacccagcagccctggccttggccgagtggccccgagaggccatcccaggcattctcactaccgcaaggacacccacagcccactggactcctctgtaaccaagcaagtgccgaggcctcctgagcggcgaagggcctgccaggatggctcagtgccacgatattctggaagctcatcgagcaccaggacacaggcctctgggaacctgagacctccctcctcgggctctcttccttcccaggaccttctgggaaccagcagtgccactgtcacccctgcagtccactcggattttgtttctggagtctccccgcacaacgctccctctgccgggtgggcaggggacgcggggtccaggacatgctcgccggcccccatacctccccacacatccgactcctgctcaaaatctggggctgccagcctgggggaagaggccagggacacgcctcagccctcctcacccttggttctgcaggttggacggcctgagcaaggggcggtgggcccccaccgaagccactgctgctcacagcctgggacgcagccggcccaagaggcccagcggggaccctcccctgaggctagctggctgtgtggcaggtactgtcccaccccgcccagggggcggccctgcccccagaggcgctcttccagctgtgggagcaccggcagcagccaccaaagcactgcccgggggccaggtgggagcccgcaggaggggacacgccagccaggccccacgccgtccccaggccccaattcaggggcatcaaggagaagcagtgccagccagggtgcggggtctcgggggctgtccgaggagaagaccttgaggagtgggggaggcccccaggggcaggaggaggccagtggtgtgtcacccagctctctgccccgctcgtctccagaggctgtggtccgcgaatggctggacaacattccagaagagcccatactcatgacatatgagttggcggacgagaccacaggtgcagctgggggtggcctgagaggccccgaggtggaccctggggatgaccattctctggaaggcctgggggagccagctcaggcgggacagcagtccctggaaggggaccccggccaggacccagagccagagggagccctcctggggagtagtgacactggtccccaatcaggagagggtgtcccccaaggggcagctccagagggagtttccgaggcccctgcagaggccggagcagacagagaggccccagcaggctgcagggtgagcctgcgggcacttcctggccgggtgtctgcctccacgcagatcatgagggcgctgatgggctccaagcagggccggcccagcagcgtgcccgaagtgtctaggcccatggccaggaggctcagctgctcagccggggccctcattacttgtctggccagtctgcagttatttgaggaagaccttgggtctcctgccagcaaagtgaggttcaaagactcccctcggtaccaggagctgctcagcatctcgaaggacctgtggccaggatgtgacgttggggaagaccagctggactcaggcctctgggagctcacatggagccaggctctgccagaccttgggtcccatgccatgacggagaacttcacgcccacatcctcctctggtgtggacatcagcagcggctctggaggctcaggggagagtagcgtaccctgtgccatggacggcaccctggtgacacaggggacagagctgcccctgaaaacctccaaccagaggcctgattcaagaacttatgagagcccaggggatctggaaaaccaacagcagtgttgtttcccaaccttcttgaacgcccgagcctgcgcttgtgccaccaatgaggatgaagcagaaagagacagtgaggagcagagggcgagctcgaacctggagcagttagctgaaaacacagtgcaagaagaggtgcaattagaggaaactaaagaaggaacagaaggagaagggctgcaagaagaggcggtgcagttagaggaaactaaaacagaagaagggctgcaagaagagggggtgcagttagaggaaactaaagaaacagaaggagaaggacagcaagaagaagaggcgcagttagaggaaattgaagaaacaggaggagaagggctgcaagaagagggggtgcagttagaggaagttaaagaagggccagaaggaggactgcaaggagaggctcttgaagagggtctcaaagaggaaggacttccagaagaaggaagcgtccacgggcaggaattgtcagaggccagctctccggatgggaaaggctcccaggaagatgacccagtccaggaggaggaagcaggaagagcctctgcctctgcagagccgtgccccgcagagggcacagaggaacccacagagccccctagtcatctcagcgagacggacccaagtgccagcgagaggcagagtggctcccagcttgagcctggtttggaaaagccgcccggagccaccatgatgggccaagagcacacgcaggcccaacccacccagggggctgcagagaggagctcttcggtggcctgcagcgcggctctggactgcgaccccatctgggtgtccgtgttactgaagaagacggagaaggccttcctggcccaccttgccagtgcggtggctgagctccgagcacgctggggcctgcaggacaatgatctgctggaccagatggcggccgagctgcagcaggacgtggcccaacgcctccaggacagcaccaagagagagctccagaagctccagggccgggcgggtaggatggtgctggagcctccaagggaggccctcaccggggagctgctcctgcagacccagcagcgcagacaccgtctccggggcctgcgaaacctctcggccttctctgagcggaccctgggcctggggcccctctccttcaccctggaggacgagccagccctcagcacagccctggggagccagctgggcgaggaggcggagggggaggagttctgtccctgcgaggcctgcgtgaggaagaaagtgagccctatgtcccccaaggccacaatgggggcaaccagaggtcccatcaaagaggcctttgacctgcagcagattctgcagaggaagaggggagaacacactgatggggaggcagcagaggtggcccctggcaagacccacacggaccccacgagcactaggactgtccagggagctgagggagggctggggccggggctgagccaggggcctggagtggacgagggtgaggatggcgaggggagccagagactcaacagagacaaagatcccaaactcggggaggcagagggagatgcaatggctcaggagagagaagggaaaacccacaacagtgaaaccagtgcgggcagtgagttgggggaagctgagcaggagggagagggcataagtgaaaggggagaaactgggggtcaaggctctgggcatgaggacaacttgcagggtgaagctgcggcaggaggtgaccaagatccaggacagagtgatggggccgaaggcatagaggccccggaggctgaaggggaggcccagccagagtcagaaggtgtagaggccccagaggcagaaggggatgcccaggaggctgaaggggaggcccagccagagtcagaagatgtagaggccccagaggcagaaggagaggcccagccagagtcagaagatgtagagaccccagaggcagaatgggaggtccagccagagtcagaaggtgcagaagccccggaggcagaaaaggaggcccagccagagacagaaagtgtagaggccctggagactgaaggggaggacgagccagagtcagaaggtgcagaggcccaagaggcagaagaggcggcccaggaggcagaagggcagacccagccagagtcagaagttatagagtcccaggaggcagaagaggaagcccagccagagtcagaagatgtagaggccctggaggttgaagtggagacccaggaggcagaaggggaggcccagccagagtcagaagatgtagaggccccagaggctgaaggggagatgcaagaggcagaagaggaggcccagccagagtcagacggtgtagaggcccagccaaagtcagaaggtgaagaggcccaggaggttgaaggggagacccagaagacagaaggggatgcccagccagagtcagacggtgtagaggccccggaggcagaagaggaggcacaggaggctgaaggggaggtccaggaggcagaaggggaggcccacccagagtcagaagatgtagatgcccaggaggcagaaggggaggcccagccagaatcagaaggtgtagaggccccagaggcagaaggggaggcccagaaggcagaaggtatagaggccccagagactgaaggggaggcccagccagagtcagaaggtatagaggccccagaggctgaaggggaggcccagcctgagtcagaaggtgtagaggcccaggatgcagaaggggaggcccagccagagtcagaaggtatagaggcccaggaggctgaagaggaggcccaaccagagttagaaggtgtagaggccccagaggcagaaggggaggcccagccagagtcagaaggtatagaggccccagaggcagaaggggaggcccaaccagagttagaaggtgtagaggccccggaggctgaagaggaggcccagccagagccagaaggcgtagagaccccggaggctgaaggggaggcccagccagagtcagaaggagaaactcaaggtgagaaaaaggggagccctcaggtcagtctaggagatggccaatctgaggaggcttctgaaagcagcagcccagtccctgaggacaggcccactccacccccttccccaggtggagacactccccaccaaaggccaggctcccaaacaggcccttcctcctccagagcatcctcttggggcaactgctggcagaaagactcagaaaatgaccatgtacttggagacacaaggagccctgatgccaagtccacggggacccctcatgcagagaggaaggccaccaggatgtacccagaaagttctacttctgagcaagaagaggcccctttgggctcaaggactccagagcagggggccagtgaaggttatgacctacaagaggaccaggcactcggaagtctcgcccccactgaggcagtgggcagggcagacggctttggccaagatgacttagatttctag

SEQ ID NO: 52 - DNA, 1053 bp, unidentified
  source 1..1053: mol_type = unassigned DNA
atgggctgcttcttctccaagagacggaaggctgacaaggagtcgcggcccgagaacgaggaggagcggccaaagcagtacagctgggatcagcgcgagaaggttgatccaaaagactacatgttcagtggactgaaggatgaaacagtaggtcgcttacctgggacggtagcaggacaacagtttctcattcaagactgtgagaactgtaacatctatatttttgatcactctgctacagttaccattgatgactgtactaactgcataatttttctgggacccgtgaaaggcagcgtgtttttccggaattgcagagattgcaagtgcacattagcctgccaacaatttcgtgtgcgagattgtagaaagctggaagtctttttgtgttgtgccactcaacccatcattgagtcttcctcaaatatcaaatttggatgttttcaatggtactatcctgaattagctttccagttcaaagatgcagggctaagtatcttcaacaatacatggagtaacattcatgactttacacctgtgtcaggagaactcaactggagccttcttccagaagatgctgtggttcaggactatgttcctatacctactaccgaagagctcaaagctgttcgtgtttccacagaagccaatagaagcattgttccaatatcccggggtcagagacagaagagcagcgatgaatcatgcttagtggtattatttgctggtgattacactattgcaaatgccagaaaactaattgatgagatggttggtaaaggctttttcctagttcagacaaaggaagtgtccatgaaagctgaggatgctcaaagggtttttcgggaaaaagcacctgacttccttcctcttctgaacaaaggtcctgttattgccttggagtttaatggggatggtgctgtagaagtatgtcaacttattgtaaacgagatattcaatgggaccaagatgtttgtatctgaaagcaaggagacggcatctggagatgtagacagcttctacaactttgctgatatacagatgggaatatga

SEQ ID NO: 53 - DNA, 3459 bp, unidentified
  source 1..3459: mol_type = unassigned DNA
atgagggagccggaagagctgatgcccgattcgggtgctgtgtttacatttgggaaaagtaaatttgctgaaaataatcccggtaaattctggtttaaaaatgatgtccctgtacatctttcatgtggagatgaacattctgctgttgttaccggaaataataaactttacatgtttggcagtaacaactggggtcagttaggattaggatcaaagtcagccatcagcaagccaacatgtgtcaaagctctaaaacctgaaaaagtgaaattagctgcctgtggaaggaaccacaccctggtgtcaacagaaggaggcaatgtatatgcaactggtggaaataatgaaggacagttggggcttggtgacaccgaagaaagaaacacttttcatgtaattagcttttttacatccgagcataagattaagcagctgtctgctggatctaatacttcagctgccctaactgaggatggaagactttttatgtggggtgacaattccgaagggcaaattggtttaaaaaatgtaagtaatgtctgtgtccctcagcaagtgaccattgggaaacctgtctcctggatctcttgtggatattaccattcagcttttgtaacaacagatggtgagctatatgtgtttggagaacctgagaatgggaagttaggtcttcccaatcagctcctgggcaatcacagaacaccccagctggtgtctgaaattccggagaaggtgatccaagtagcctgtggtggagagcatactgtggttctcacggagaatgctgtgtatacctttgggctgggacaatttggtcagctgggtcttggcacttttctttttgaaacttcagaacccaaagtcattgagaatattagggatcaaacaataagttatatttcttgtggagaaaatcacacagctttgataacagatatcggccttatgtatacttttggagatggtcgccacggaaaattaggacttggactggagaattttaccaatcacttcattcctactttgtgctctaattttttgaggtttatagttaaattggttgcttgtggtggatgtcacatggtagtttttgctgctcctcatcgtggtgtggcaaaagaaattgaattcgatgaaataaatgatacttgcttatctgtggcgacttttctgccgtatagcagtttaacctcaggaaatgtactgcagaggactctatcagcacgtatgcggcgaagagagagggagaggtctccagattctttttcaatgaggagaacactacctccaatagaagggactcttggcctttctgcttgttttctccccaattcagtctttccacgatgttctgagagaaacctccaagagagtgtcttatctgaacaggacctcatgcagccagaggaaccagattatttgctagatgaaatgaccaaagaagcagagatagataattcttcaactgtagaaagccttggagaaactactgatatcttaaacatgacacacatcatgagcctgaattccaatgaaaagtcattaaaattatcaccagttcagaaacaaaagaaacaacaaacaattggggaactgacgcaggatacagctcttactgaaaacgatgatagtgatgaatatgaagaaatgtcagaaatgaaagaagggaaagcatgtaaacaacatgtgtcacaagggattttcatgacgcagccagctacgactatcgaagcattttcagatgaggaagtagagatcccagaggagaaggaaggagcagaggattcaaaaggaaatggaatagaggagcaagaggtagaagcaaatgaggaaaatgtgaaggtgcatggaggaagaaaggagaaaacagagatcctatcagatgaccttacagacaaagcagaggtgagtgaaggcaaggcaaaatcagtgggagaagcagaggatgggcctgaaggtagaggggatggaacctgtgaggaaggtagttcaggagcagaacactggcaagatgaggagagggagaagggggagaaagacaagggtagaggagaaatggagaggccaggagagggagagaaggaactagcagagaaggaagaatggaagaagagggatggggaagagcaggagcaaaaggagagggagcagggccatcagaaggaaagaaaccaagagatggaggagggaggggaggaggagcatggagaaggagaagaagaggagggagacagagaagaggaagaagagaaggagggagaagggaaagaggaaggagaaggggaagaagtggagggagaacgtgaaaaggaggaaggagagaggaaaaaggaggaaagagcggggaaggaggagaaaggagaggaagaaggagaccaaggagagggggaagaggaggaaacagaggggagaggggaggaaaaagaggagggaggggaagtagagggaggggaagtagaggaggggaaaggagagagggaagaggaagaggaggagggtgagggggaagaggaggaaggggagggggaagaggaggaaggggagggggaagaggaggaaggagaagggaaaggggaggaagaaggggaagaaggagaaggggaggaagaaggggaggaaggagaaggggagggggaagaggaggaaggagaaggggagggagaagaggaaggagaaggggagggagaagaggaggaaggagaaggggagggagaagaggaaggagaaggggagggagaagaggaggaaggagaagggaaaggggaggaggaaggagaggaaggagaaggggagggggaagaggaggaaggagaaggggaaggggaggatggagaaggggagggggaagaggaggaaggagaatgggagggggaagaggaggaaggagaaggggagggggaagaggaaggagaaggggaaggggaggaaggagaaggggagggggaagaggaggaaggagaaggggagggggaagaggaggaaggggaagaagaaggggaggaagaaggagagggagaggaagaaggggagggagaaggggaggaagaagaggaaggggaagtggaaggggaggtggaaggggaggaaggagagggggaaggagaggaagaggaaggagaggaggaaggagaagaaagggaaaaggagggggaaggagaagaaaacaggaggaacagagaagaggaggaggaagaagaggggaagtatcaggagacaggcgaagaagagaatgaaaggcaggatggagaggagtacaaaaaagtgagcaaaataaaaggatctgtgaaatatggcaaacataaaacatatcaaaaaaagtcagttactaacacacagggaaatgggaaagagcagaggtccaaaatgccagtccagtcaaaacgacttttaaaaaacgggccatcaggttccaaaaagttctggaataatgtattaccacattacttggaattgaagtaa

SEQ ID NO: 54 - DNA, 1929 bp, unidentified
  source 1..1929: mol_type = unassigned DNA
atgagggagccggaagagctgatgcccgattcgggtgctgtgtttacatttgggaaaagtaaatttgctgaaaataatcccggtaaattctggtttaaaaatgatgtccctgtacatctttcatgtggagatgaacattctgctgttgttaccggaaataataaactttacatgtttggcagtaacaactggggtcagttaggattaggatcaaagtcagccatcagcaagccaacatgtgtcaaagctctaaaacctgaaaaagtgaaattagctgcctgtggaaggaaccacaccctggtgtcaacagaaggaggcaatgtatatgcaactggtggaaataatgaaggacagttggggcttggtgacaccgaagaaagaaacacttttcatgtaattagcttttttacatccgagcataagattaagcagctgtctgctggatctaatacttcagctgccctaactgaggatggaagactttttatgtggggtgacaattccgaagggcaaattggtttaaaaaatgtaagtaatgtctgtgtccctcagcaagtgaccattgggaaacctgtctcctggatctcttgtggatattaccattcagcttttgtaacaacagatggtgagctatatgtgtttggagaacctgagaatgggaagttaggtcttcccaatcagctcctgggcaatcacagaacaccccagctggtgtctgaaattccggagaaggtgatccaagtagcctgtggtggagagcatactgtggttctcacggagaatgctgtgtatacctttgggctgggacaatttggtcagctgggtcttggcacttttctttttgaaacttcagaacccaaagtcattgagaatattagggatcaaacaataagttatatttcttgtggagaaaatcacacagctttgataacagatatcggccttatgtatacttttggagatggtcgccacggaaaattaggacttggactggagaattttaccaatcacttcattcctactttgtgctctaattttttgaggtttatagttaaattggagaggtctccagattctttttcaatgaggagaacactacctccaatagaagggactcttggcctttctgcttgttttctccccaattcagtctttccacgatgttctgagagaaacctccaagagagtgtcttatctgaacaggacctcatgcagccagaggaaccagattatttgctagatgaaatgaccaaagaagcagagatagataattcttcaactgtagaaagccttggagaaactactgatatcttaaacatgacacacatcatgagcctgaattccaatgaaaagtcattaaaattatcaccagttcagaaacaaaaggatcatgaattttctaaaactgaggaactaaaactagaagatgtggatgaggaaattaatgctgaaaatgtggaaagcaagaagaaaactgtgggagatgatgaaagtgttcctacaggttatcacagtaaaacagaaggagcagaaagaaccaatgatgatagctcagctgaaactattgaaaagaaagaaaaagccaacctagaggaacgggccatttgtgagtacaatgaaaacccaaaaggatacatgcttgatgatgcagatagcagttcattagaaatcctagaaaacagtgaaacaacaccaagcaaagacatgaaaaaaacaaagaagatttttctgttcaaaagagtcccctcaataaatcaaaagattgtcaagaataacaatgagccgctcccagagataaaatccataggagaccagatcattttaaaaagtgataataaagatgccgaccagaaccacatgagtcagaatcatcagaatatcccaccaacaaatacagagagaagatcaaaatcctgtacaatactataa

SEQ ID NO: 55 - DNA, 3861 bp, unidentified
  source 1..3861: mol_type = unassigned DNA
atgtcacatctggtggaccctacatcaggagacttgccagttagagacatagatgctatacctctggtgctaccagcctcaaaaggtaagaatatgaaaactcaaccacccttgagcaggatgaaccgggaggaattggaggacagtttctttcgacttcgcgaagatcacatgttggtgaaggagctttcttggaagcaacaggatgagatcaaaaggctgaggaccaccttgctgcggttgaccgctgctggccgggacctgcgggtcgcggaggaggcggcgccgctctcggagaccgcaaggcgcgggcagaaggcgggatggcggcagcgcctctccatgcaccagcgcccccagatgcaccgactgcaagggcatttccactgcgtcggccctgccagcccccgccgcgcccagcctcgcgtccaagtgggacacagacagctccacacagccggtgcaccggtgccggagaaacccaagagggggccaagggacaggctgagctacacagcccctccatcgtttaaggagcatgcgacaaatgaaaacagaggtgaagtagccagtaaacccagtgaacttgtttctggttctaacagcataatttctttcagcagtgtcataagtatggctaaacccattggtctatgcatgcctaacagtgcccacatcatggccagcaataccatgcaagtggaagagccacccaagtctcctgagaaaatgtggcctaaagatgaaaattttgaacagagaagctcattggagtgtgctcagaaggctgcagagcttcgagcttccattaaagagaaggtagagctgattcgacttaagaagctcttacatgaaagaaatgcttcattggttatgacaaaagcacaattaacagaagttcaagaggcatacgaaaccttgctccagaagaatcagggaatcctgagtgcagcccatgaggccctcctcaagcaagtgaatgagctcagggcagagctgaaggaagaaagcaagaaggctgtgagcttgaagagccaactggaagatgtgtctatcttgcagatgactctgaaggagtttcaggagagagttgaagatttggaaaaagaacgaaaattgctgaatgacaattatgacaaactcttagaaagcatgctggacagcagtgacagctccagtcagccccactggagcaacgagctcatagcggaacagctacagcagcaagtctctcagctgcaggatcagctggatgctgagctggaggacaagagaaaagttttacttgagctgtccagggagaaagcccaaaatgaggatctgaagcttgaagtcaccaacatacttcagaagcataaacaggaagtagagctcctccaaaatgcagccacaatttcccaacctcctgacaggcaatctgaaccagccactcacccagctgtattgcaagagaacactcagatcgagccaagtgaacccaaaaaccaagaagaaaagaaactgtcccaggtgctaaatgagttgcaagtatcacacgcagagaccacattggaactagaaaagaccagggacatgcttattctgcagcgcaaaatcaacgtgtgttatcaggaggaactggaggcaatgatgacaaaagctgacaatgataatagagatcacaaagaaaagctggagaggttgactcgactactagacctcaagaataaccgtatcaagcagctggaaggtattttaagaagccatgaccttccaacatctgaacagctcaaagatgttgcttatggcacccgaccgttgtcgttatgtttggaaacactgccagcccatggagatgaggataaagtggatatttctctgctgcatcagggtgagaatctttttgaactgcacatccaccaggccttcctgacatctgccgccctagctcaggctggagatacccaacctaccactttctgcacctattccttctatgactttgaaacccactgtaccccattatctgtggggccacagcccctctatgacttcacctcccagtatgtgatggagacagattcgcttttcttacactaccttcaagaggcttcagcccggcttgacatacaccaggccatggccagtgaacacagcactcttgctgcaggatggatttgctttgacagggtgctagagactgtggagaaagtccatggcttggccacactgattggagctggtggagaagagttcggggttctagagtactggatgaggctgcgtttccccataaaacccagcctacaggcgtgcaataaacgaaagaaagcccaggtctacctgtcaaccgatgtgcttggaggccggaaggcccaggaagaggagttcagatcggagtcttgggaacctcagaacgagctgtggattgaaatcaccaagtgctgtggcctccggagtcgatggctgggaactcaacccagtccatatgctgtgtaccgcttcttcaccttttctgaccatgacactgccatcattccagccagtaacaacccctactttagagaccaggctcgattcccagtgcttgtgacctctgacctggaccattatctgagacgggaggccttgtctatacatgtttttgatgatgaagacttagagcctggctcgtatcttggccgagcccgagtgcctttactgcctcttgcaaaaaatgaatctatcaaaggtgattttaacctcactgaccctgcagagaaacccaacggatctattcaagtgcaactggattggaagtttccctacataccccctgagagcttcctgaaaccagaagctcagactaaggggaaggataccaaggacagttcaaagatctcatctgaagaggaaaaggcttcatttccttcccaggatcagatggcatctcctgaggttcccattgaagctggccagtatcgatctaagagaaaacctcctcatgggggagaaagaaaggagaaggagcaccaggttgtgagctactcaagaagaaaacatggcaaaagaataggtgttcaaggaaagaatagaatggagtatcttagccttaacatcttaaatggaaatacaccagagcaggtgaattacactgagtggaagttctcagagactaacagcttcataggtgatggctttaaaaatcagcacgaggaagaggaaatgacattatcccattcagcactgaaacagaaggaacctctacatcctgtaaatgacaaagaatcctctgaacaaggttctgaagtcagtgaagcacaaactaccgacagtgatgatgtcatagtgccacccatgtctcagaaatatcctaaggcagattcagagaagatgtgcattgaaattgtctccctggccttctacccagaggcagaagtgatgtctgatgagaacataaaacaggtgtatgtggagtacaaattctacgacctacccttgtcggagacagagactccagtgtccctaaggaagcctagggcaggagaagaaatccactttcactttagcaaggtaatagacctggacccacaggagcagcaaggccgaaggcggtttctgttcgacatgctgaatggacaagatcctgatcaaggacatttaaagtttacagtggtaagtgatcctctggatgaagaaaagaaagaatgtgaagaagtgggatatgcatatcttcaactgtggcagatcctggagtcaggaagagatattctagagcaagagctagacattgttagccctgaagatctggctaccccaataggaaggctgaaggtttcccttcaagcagctgctgtcctccatgctatttacaaggagatgactgaagatttgttttcatga

SEQ ID NO: 56 - DNA, 2787 bp, unidentified
  source 1..2787: mol_type = unassigned DNA
atgctgaaactggataataaagacgtcatatcacaccccttggggtatccatctgagagcttgctttccattgccagcatgctggacagcagtgacagctccagtcagccccactggagcaacgagctcatagcggaacagctacagcagcaagtctctcagctgcaggatcagctggatgctgagctggaggacaagagaaaagttttacttgagctgtccagggagaaagcccaaaatgaggatctgaagcttgaagtcaccaacatacttcagaagcataaacaggaagtagagctcctccaaaatgcagccacaatttcccaacctcctgacaggcaatctgaaccagccactcacccagctgtattgcaagagaacactcagatcgagccaagtgaacccaaaaaccaagaagaaaagaaactgtcccaggtgctaaatgagttgcaagtatcacacgcagagaccacattggaactagaaaagaccagggacatgcttattctgcagcgcaaaatcaacgtgtgttatcaggaggaactggaggcaatgatgacaaaagctgacaatgataatagagatcacaaagaaaagctggagaggttgactcgactactagacctcaagaataaccgtatcaagcagctggaaggtattttaagaagccatgaccttccaacatctgaacagctcaaagatgttgcttatggcacccgaccgttgtcgttatgtttggaaacactgccagcccatggagatgaggataaagtggatatttctctgctgcatcagggtgagaatctttttgaactgcacatccaccaggccttcctgacatctgccgccctagctcaggctggagatacccaacctaccactttctgcacctattccttctatgactttgaaacccactgtaccccattatctgtggggccacagcccctctatgacttcacctcccagtatgtgatggagacagattcgcttttcttacactaccttcaagaggcttcagcccggcttgacatacaccaggccatggccagtgaacacagcactcttgctgcaggatggatttgctttgacagggtgctagagactgtggagaaagtccatggcttggccacactgattggagctggtggagaagagttcggggttctagagtactggatgaggctgcgtttccccataaaacccagcctacaggcgtgcaataaacgaaagaaagcccaggtctacctgtcaaccgatgtgcttggaggccggaaggcccaggaagaggagttcagatcggagtcttgggaacctcagaacgagctgtggattgaaatcaccaagtgctgtggcctccggagtcgatggctgggaactcaacccagtccatatgctgtgtaccgcttcttcaccttttctgaccatgacactgccatcattccagccagtaacaacccctactttagagaccaggctcgattcccagtgcttgtgacctctgacctggaccattatctgagacgggaggccttgtctatacatgtttttgatgatgaagacttagagcctggctcgtatcttggccgagcccgagtgcctttactgcctcttgcaaaaaatgaatctatcaaaggtgattttaacctcactgaccctgcagagaaacccaacggatctattcaagtgcaactggattggaagtttccctacataccccctgagagcttcctgaaaccagaagctcagactaaggggaaggataccaaggacagttcaaagatctcatctgaagaggaaaaggcttcatttccttcccaggatcagatggcatctcctgaggttcccattgaagctggccagtatcgatctaagagaaaacctcctcatgggggagaaagaaaggagaaggagcaccaggttgtgagctactcaagaagaaaacatggcaaaagaataggtgttcaaggaaagaatagaatggagtatcttagccttaacatcttaaatggaaatacaccagagcaggtgaattacactgagtggaagttctcagagactaacagcttcataggtgatggctttaaaaatcagcacgaggaagaggaaatgacattatcccattcagcactgaaacagaaggaacctctacatcctgtaaatgacaaagaatcctctgaacaaggttctgaagtcagtgaagcacaaactaccgacagtgatgatgtcatagtgccacccatgtctcagaaatatcctaaggcagattcagagaagatgtgcattgaaattgtctccctggccttctacccagaggcagaagtgatgtctgatgagaacataaaacaggtgtatgtggagtacaaattctacgacctacccttgtcggagacagagactccagtgtccctaaggaagcctagggcaggagaagaaatccactttcactttagcaaggtaatagacctggacccacaggagcagcaaggccgaaggcggtttctgttcgacatgctgaatggacaagatcctgatcaaggacatttaaagtttacagtggtaagtgatcctctggatgaagaaaagaaagaatgtgaagaagtgggatatgcatatcttcaactgtggcagatcctggagtcaggaagagatattctagagcaagagctagacattgttagccctgaagatctggctaccccaataggaaggctgaaggtttcccttcaagcagctgctgtcctccatgctatttacaaggagatgactgaagatttgttttcatga

SEQ ID NO: 57 - DNA, 1800 bp, unidentified
  source 1..1800: mol_type = unassigned DNA
atggatggcagccggagagtcagagcaacctctgtccttcccagatatggtccaccgtgcctatttaaaggacacttgagcaccaaaagtaatgctttttgcactgactcctcttctctcagactaagcactctccagctggtcaagaatcacatggctgttcactataataaaatcctttcagccaaagctgcagtagactgctcggttccagtaagcgtgagtaccagcataaagtatgcagaccaacaacgaagagagaaactcaaaaaggaattagcacaatgtgaaaaagagttcaaattaactaaaactgcaatgcgagccaattataaaaataattccaagtcactttttaataccttacaaaagccctcaggcgaaccgcaaattgaggatgacatgttaaaagaagaaatgaatggattttcatcctttgcaaggtcactagtaccctcttcagagagactacacctaagtctacataaatccagtaaagtcatcacaaatggtcctgagaagaactccagttcctccccgtccagtgtggattatgcagcctccgggccccggaaactgagctctggagccctgtatggcagaaggcccagaagcacattcccaaattcccaccggtttcagttagtcatttcgaaagcacccagtggggatcttttggataaacattctgaactcttttctaacaaacaattgccattcactcctcgcactttaaaaacagaagcaaaatctttcctgtcacagtatcgctattatacacctgccaaaagaaaaaaggattttacagatcaacggatagaagctgaaacccagactgaattaagctttaaatctgagttggggacagctgagactaaaaacatgacagattcagaaatgaacataaagcaggcatctaattgtgtgacatatgatgccaaagaaaaaatagctcctttacctttagaagggcatgactcaacatgggatgagattaaggatgatgctcttcagcattcctcaccaagggcaatgtgtcagtattccctgaagcccccttcaactcgtaaaatctactctgatgaagaagaactgttgtatctgagtttcattgaagatgtaacagatgaaattttgaaacttggtttattttcaaacaggtttttagaacgactgttcgagcgacatataaaacaaaataaacatttggaggaggaaaaaatgcgccacctgctgcatgtcctgaaagtagacttaggctgcacatcggaggaaaactcggtaaagcaaaatgatgttgatatgttgaatgtatttgattttgaaaaggctgggaattcagaaccaaatgaattaaaaaatgaaagtgaagtaacaattcagcaggaacgtcaacaataccaaaaggctttggatatgttattgtcggcaccaaaggatgagaacgagatattcccttcaccaactgaatttttcatgcctatttataaatcaaagcattcagaaggggttataattcaacaggtgaatgatgaaacaaatcttgaaacttcaactttggatgaaaatcatccaagtatttcagacagtttaacagatcgggaaacttctgtgaatgtcattgaaggtgatagtgaccctgaaaaggttgagatttcaaatggattatgtggtcttaacacatcaccctcccaatctgttcagttctccagtgtcaaaggcgacaataatcatgacatggagttatcaactcttaaaatcatggaaatgagcattgaggactgccctttggatgtttaa

SEQ ID NO: 58 - DNA, 3138 bp, unidentified
  source 1..3138: mol_type = unassigned DNA
atggggtcgcagccgccgctggggtctccgctgtctcgcgaggagggtgaagcgcccccgcctgctcccgcttcggagggtaggcggagaagtcgccgggtacgccttcgcggatcctgccgacaccgacctagctttctgggctgccgggagctcgcggcgagcgccccagccaggcctgcgccggcatcctccgagataatggcatcagctgctaaggaatttaaaatggacaacttttcacctaaagctggcactagcaaattgcaacagacagtaccagctgatgcatctcctgattctaagtgtcctatatgcttggatagatttgataatgtgtcttacttagatcgctgcttacataagttctgttttcgctgtgtacaggagtggtcaaaaaacaaagctgaatgcccactatgtaaacagccctttgattctattttccattctgtgagggcagaagatgacttcaaggagtatgtcctaaggccttcgtataatggttcttttgtcacccctgatcgacgatttcgctaccgtacaactctgacaagggaacgaaatgcttctgtgtattcacctagtggtcctgtgaacagaagaacaacaactccaccggatagtggagtactgtttgaagggttaggcatttcaacaagacctagagatgttgaaattcctcagtttatgagacagattgcagtaaggaggccaactacggcagatgaaagatctttgcggaaaattcaagaacaagatattattaattttagacgaactctttatcgtgctggtgctcgagttagaaatattgaagatggtggccgctacagggatatttcagctgaatttttccgtagaaatccagcttgccttcacagattagtcccctggttaaaacgtgaacttacagttctttttggagctcatggatctttagtgaatattgtccagcatattatcatgagtaatgttactcgctatgacttggagagtcaggcatttgtgtctgatttaagaccatttttacttaatcgaactgagcattttatacatgaatttatcagttttgcccgatctccttttaacatggcagcctttgaccagcatgccaattatgattgccctgctccttcatacgaagaaggcagccattctgattcttcagtcataacaatatctccagatgaggctgagacccaagagctggatattaatgtagccactgttagtcaggcaccatgggatgatgaaactccaggaccatcttactcaagctcagagcaggtacacgttactatgtcttctcttttaaatacttctgacagttcagatgaagaacttgtcacaggaggagccacgtctcagatacaaggagtacaaaccaatgacgacctaaataatgacagtgatgattcttcagataattgtgtcattgttgggtttgttaaaccactagctgagaggaccccagaacttgttgaactgtcctctgattctgaggacttaggttcttatgagaaaatggagacagtgaagacacaagaacaggagcaatcttacagttctggtgatagcgatgttagtagatgctcatctccacactctgtccttggaaaggatgaacaaataaataaaggtcattgtgattctagtacaagaatcaaatcaaagaaggaagagaaacgatctacatcattgtcatctcccagaaacctgaactcatctgtaagaggagacagagtatattctccatataaccatagacacagaaagaggggaagatcaagaagttcagattcacgttctcagagtagaagtgggcatgatcagaagaatcatagaaagcatcatgggaagaaaagaatgaaaagtaaacgatccagaagcagggaaagtagcagacctagagggagaagagacaaaaagagatcaagaactagagatagcagttggtccagaagaagccaaactctgtctctaagtagtgaaagcacaagcagatcaaggtctcgtagcagtgatcatggtaaaagaagatcacggagcagaaatagagatcgttattatttaagaaataattatggaagcagatacaaatgggagtatacttattacagtagaaacaaggacagggatgggtacgaatcatcttacaggaggaggactctgtccagagctcattattctagacagtcttcaagtccagaatttagagttcagtccttttctgaaagaacaaatgctaggaaaaaaaataatcacagtgagaggaagtattactactatgaaaggcacagatcaaggagcctgtctagtaacagatcaaggactgcatctaccgggactgaccgggtgagaaatgaaaagcctggagggaaacgaaaatacaaaacacggcatttggagggtactaacgaagtggctcagccatctcgtgaatttgcttctaaagcaaaggacagtcattaccaaaaatcttcatcaaaattggatggaaactacaaaaatgagagtgataccttttcagacagccgatcatcagacagagagacaaaacacaaaaggagaaaaaggaagacccggagcctaagtgtagagatagtttatgaaggaaaagctactgatacaactaaacaccataaaaagaaaaagaagaaacataagaagaagcataagaaacaccatggagataatgcttcacgttccccagttgtaattaccattgacagtgacagtgataaggattctgaagtaaaggaggatacagaatgtgacaatagtggtcctcaagaccctctacaaaatgagtttttggctccttccttggaaccatttgaaactaaagatgtagttacaatagaagctgaatttggtgtgctggacaaggaatgtgatattgccacacttagtaacaacttgaataatgccaacaaaactgtagataatattccacctctggcagcttcagttgaacaaactctcgatgtaagagaagagagcacctttgtttctgatttggagaaccagcccagtaacattgtgtctcttcaaactgagccatcaaggcaattgccatcgccacggacatcattaatgtcagtatgtcttggtagagactgtgatatgtcttaa

SEQ ID NO: 59 - DNA, 1548 bp, unidentified
  source 1..1548: mol_type = unassigned DNA
atgagctcggagatggagccgctgctcctggcctggagctattttaggcgcaggaagttccagctctgcgccgatctatgcacgcagatgctggagaagtccccttatgaccaggaaccagatcctgaattgccagtgcatcaggcagcttggatcttaaaagcaagagcgctaacagaaatggtatacatagatgaaattgatgtagatcaggaaggaattgcagaaatgatgctggatgaaaatgctatagctcaagttccacgccctggaacgtctttgaaactccctggaactaatcagacaggagggcctagccaggccgttaggccaatcacacaagctggaagacccattacaggtttcctcaggcccagcacgcagagtggaaggccaggcactatggaacaggctatcagaacacccagaaccgcctacacagcccgccctatcaccagctcctccggaagatttgtcaggctgggaacggcttccatgcttacaagtcctgatggaccatttataaatttatctaggctgaatttaacaaagtattcccagaaacctaagttggcaaaggctttgtttgagtatatctttcatcatgaaaatgatgttaagactgctttggatctggctgccctctccacagaacattctcagtacaaggactggtggtggaaagtacagattggaaaatgttactacaggttgggaatgtatcgtgaagcagaaaaacagtttaaatcagccctgaagcagcaggaaatggtagatacatttctgtacttggcaaaagtttatgtctcattggatcaacctgtgactgctttaaatcttttcaaacaaggcttagataagtttccaggagaagtaaccctgctctgtggaattgcaagaatctatgaggaaatgaacaatatgtcatcagcagcagaatattacaaagaagttttgaaacaagacaatactcatgtggaagccatcgcatgcattggaagcaaccacttctattctgatcagccagaaatagctctccggttttacaggcggctgctgcagatgggcatttataacggccagctttttaacaatctggggctgtgttgcttctatgcccagcagtatgatatgactctgacctcatttgaacgtgccctttctttggctgaaaatgaagaagaggcagctgatgtctggtacaacttgggacatgtagctgtgggaataggagatacaaatttggcccatcagtgcttcaggctggctctggtcaacaacaacaaccacgccgaggcctacaacaacctggctgtgctggagatgcggaagggccacgttgaacaggcaagggcactattacaaactgcatcatcattagcaccccatatgtatgaaccgcattttaattttgcaacaatctctgataagattggagatctgcagagaagctatgttgctgcgcagaagtctgaagcagcatttccagaccatgtggacacacaacatttaattaaacaattaaggcagcattttgctatgctctga

SEQ ID NO: 60 - DNA, 15609 bp, unidentified
  source 1..15609: mol_type = unassigned DNA
atgaattgcccagttctttcattgggctctggcttcttgtttcaggtcattgaaatgttgatctttgcctattttgcttcaatatccttgactgagtcacgaggtcttttcccaaggctggagaacgtgggagctttcaagaaagtttccatcgtgccaacccaagcagtatgtggactcccagaccgaagcactttttgtcacagctctgctgctgctgaaagtattcagttctgtacccagcggttttgtattcaggattgcccatacagatcttcacaccctacctacactgcccttttctcagcaggcctcagtagctgcatcacaccagacaagaatgatctgcatcctaacgcccatagcaattctgcaagttttatttttggaaatcacaagagctgcttttcttctcctccttctccaaagctgatggcatcatttaccttagctgtatggctgaaacctgagcaacaaggtgtaatgtgtgttatagaaaagacagtagatgggcagattgtgttcaaacttacaatatctgagaaagagaccatgttttattatcgcacagtaaatggtttgcaacctccaataaaagtaatgacactggggagaattcttgtgaagaaatggattcatcttagtgtgcaggtgcatcagacaaaaatcagcttctttatcaatggcgtggagaaggatcatacacctttcaatgcaagaactctaagtggttcaattacagattttgcatctggtactgtgcaaataggacagagtttaaatggtttagagcagtttgtcggaagaatgcaagattttcgattataccaagtggcacttacaaacagagagattctggaagtcttctctggagatcttctcagattgcatgcccaatcacattgccgttgccctggcagccacccgcgggtccaccctttggcacagcggtactgcattcctaatgatgcaggagacacagctgataatagagtgtcacggttgaatcctgaagcccatcctctctcttttgtcaatgataatgatgttggtacttcatgggtttcaaatgtgtttacaaacattacacagcttaatcaaggagtgactatttcagttgatttggaaaatggacagtatcaggtgttttatattatcattcagttctttagtccacaaccaacggaaataaggattcaaaggaagaaggaaaatagtttagattgggaggactggcaatattttgccaggaattgtggtgcttttggaatgaaaaacaatggagatttggaaaaacctgattctgtcaactgtcttcagctttccaattttactccatattcccgtggcaatgtcacatttagcatcctgacacctggaccaaattatcgtcctggatacaataacttctataataccccatctcttcaagagttcgtaaaagccacgcaaataaggtttcattttcatgggcagtactatacaactgagactgctgttaacctcagacacagatattatgcagtggacgaaatcaccattagtgggagatgtcagtgccatggtcatgccgataactgcgacacaacaagccagccatatagatgcctctgctcccaggagagcttcactgaaggacttcattgtgatcgctgcttgcctctttataatgacaagcctttccgccaaggtgatcaagtttacgctttcaattgtaaaccttgtcaatgcaacagccattccaaaagctgccattacaacatctctgtagacccatttccttttgagcacttcagagggggaggaggagtttgtgatgattgtgagcataacactacaggaaggaactgtgagctgtgcaaggattactttttccgacaagttggtgcagatccttcggccatagatgtttgcaaaccctgtgactgtgatacagttggcactagaaatggtagcattctttgtgatcagattggaggacagtgtaattgtaagagacacgtgtctggcaggcagtgcaatcagtgccagaatggattctacaatctacaagagttggatcctgatggctgcagtccctgtaactgcaatacctctgggacagtggatggagatattacctgtcaccaaaattcaggccagtgcaagtgcaaagcaaacgttattgggcttaggtgtgatcattgcaattttggatttaaatttctccgaagctttaatgatgttggatgtgagccctgccagtgtaacctccatggctcagtgaacaaattctgcaatcctcactctgggcagtgtgagtgcaaaaaagaagccaaaggacttcagtgtgacacctgcagagaaaacttttatgggttagatgtcaccaattgtaaggcctgtgactgtgacacagctggatccctccctgggactgtctgtaatgctaagacagggcagtgcatctgcaagcccaatgttgaagggagacagtgcaataaatgtttggagggaaacttctacctacggcaaaataattctttcctctgtctgccttgcaactgtgataagactgggacaataaatggctctctgctgtgtaacaaatcaacaggacaatgtccttgcaaattaggggtaacaggtcttcgctgtaatcagtgtgagcctcacaggtacaatttgaccattgacaattttcaacactgccagatgtgtgagtgtgattccttggggacattacctgggaccatttgtgacccaatcagtggccagtgcctgtgtgtgcctaatcgtcaaggaagaaggtgtaatcagtgtcaaccaggtttttatatttctccaggcaatgccactggctgcctgccatgctcatgccatacaactggtgcagttaatcacatctgtaatagcctgactggtcagtgtgtttgccaagatgcttccattgctgggcaacgttgtgaccaatgcaaagaccattactttggatttgatcctcagactggaagatgtcagccttgtaattgtcatctctcaggagccttgaatgaaacctgtcacttggtcacaggccagtgtttctgtaaacaatttgtcactggctcaaagtgtgatgcttgtgttcccagtgcaagccacttggatgtcaacaatctattgggttgcagcaaaactccattccagcaacctccgcccagaggacaagttcaaagttcttctgctatcaatctctcctggagtccacctgattctccaaatgcccactggcttacttacagtttactcagggatggttttgaaatctacacaacagaggatcaatacccatacagtattcaatacttcttagacacagacctgttaccatataccaaatattcctattacattgagaccaccaatgtgcatggttcaacaaggagtgtagctgtcacttacaagacaaaaccaggggtcccagagggaaacttgactttaagttatatcattcctattggctcagactctgtgacacttacctggacaacactctcaaatcaatctggtcccatagagaaatatattttgtcctgtgcccctttggctggtggtcagccatgtgtttcctacgaaggtcatgaaacctcagctaccatctggaatctggttccatttgccaagtacgatttttctgtacaggcgtgtactagcgggggctgtttacacagcttgcccattacagtgaccacagcccaggcccctccccaaagactaagtccacctaagatgcagaaaatcagttctacagaacttcatgtagaatggtctccaccagcggaactaaatggaataattataagatatgaactatacatgagaagactgagatctactaaagaaaccacatctgaggaaagtcgagtttttcagagcagtggttggctcagtcctcattcatttgtagaatcggccaatgaaaatgcattaaaacctcctcaaacaatgacaaccatcactggcttggagccatacaccaagtatgagttcagagtcttagctgtgaatatggctggaagtgtgtcttctgcctgggtctcagaaagaacgggagaatcagcacctgtattcatgatccctccttcagtctttcccctctcttcgtactctctcaatatctcctgggagaagccagcagataatgttacaagaggaaaagttgtggggtatgacatcaatatgctttctgaacaatcacctcaacagtctattcccatggcgttttcacagctgttgcacactgctaaatcccaagaactatcttacactgtagaaggactgaaaccttataggatatatgagtttactattactctctgcaattcagttggttgtgtgaccagtgcttcgggagcaggacaaactttagcagcagcaccagcacaactgaggccacctctggttaaaggaatcaacagcacaacaatccatcttaggtggtttccacctgaagaactgaatggaccctctcctatatatcagctggaaaggagagagtcatctctaccagctctgatgaccacgatgatgaaaggaatccgtttcataggaaatgggtattgtaaatttcccagctccactcacccagtcaatacagacttcactggcattaaggccagctttcgaacaaaagtgcctgaaggtttgattgtctttgcagcatcacctggcaatcaggaagagtattttgcacttcagttgaagaagggacgtctttattttctttttgatcctcaggggtcaccagtggaagtaactacaactaatgatcatggcaaacaatatagtgatggaaaatggcatgaaataattgctattaggcatcaggcttttggccaaatcactctggatgggatatatacaggttcctctgccatcctgaatggtagtactgttattggagataacacaggagtctttctgggagggctcccgcgaagttataccatcctcaggaaggatcctgagataatccaaaaaggttttgtgggctgtctcaaggatgtacattttatgaagaattacaatccgtcagctatttgggaacctctggattggcagagttctgaagaacaaatcaacgtgtataacagctgggagggatgtcccgcttcattaaatgagggagctcagttcctaggagcagggttcctggaacttcatccatatatgtttcatggtggaatgaactttgagatttcctttaagttcagaactgaccaattaaatggattgcttcttttcgtttataacaaagatggacctgattttcttgctatggagctgaaaagtggaatattgaccttccggttaaataccagtcttgcctttacacaagtggatctattgctggggctatcctattgtaatggaaagtggaataaagtcattattaaaaaggaaggctctttcatatcagcaagtgtgaatggactgatgaagcatgcatcggagtccggagaccagccactggtggtgaattcaccagtttatgtgggaggaatcccacaggaactgctgaactcttatcaacatttgtgtttggaacaaggtttcggtggttgcatgaaggatgttaaatttacacggggtgctgtcgttaacttggcatctgtgtccagcggtgctgtcagagtcaatctggatggatgcctatcaactgacagtgctgttaactgcaggggaaatgactccatcctggtttaccagggaaaagagcagagtgtttacgagggtggtctccagccttttacagaatacctgtatcgagtgatagcctcgcatgaaggaggttcagtatatagtgattggagtcgaggacgtacaacaggagcagctccacaaagtgtgccaactccctcaagagtccgcagcttaaatggatacagcattgaggtgacctgggatgaacctgttgtcagaggtgtaattgagaagtacattctgaaagcctatagtgaggacagcacccgtccaccccgcatgccctctgccagtgctgaatttgtcaatacaagcaacctcacaggcatattgacaggcttgctacccttcaaaaactatgcagtaaccctaactgcttgcactttggctggctgtactgagagctcacatgcattgaacatctctactccacaagaagccccacaagaggttcagccaccagtagccaaatcccttcccagttctttgctgctctcctggaacccacccaaaaaggcaaatggtattataactcagtactgtttatacatggatgggaggctgatctattcaggcagtgaggagaactacatagtcacagatttagcagtatttacaccccaccagtttctactaagtgcatgcacacatgtgggctgtacaaacagttcctgggtcctactgtacacagcacagctgccaccagaacacgtggattccccagttctgactgtcctggattctagaactatacacatacagtggaaacaaccaagaaaaataagtgggattctggaacgctatgtattatatatgtcaaaccatacacatgattttacaatttggagtgtcatctataacagtacagaacttttccaggatcatatgctacaatacgttttacctggtaataaatatctcatcaagctgggagcttgcacaggtggtgggtgcacagtgagtgaggccagtgaggccctaactgacgaggacatacccgaaggcgtgccagcccccaaagcccactcatattcacctgactcctttaatgtctcctggactgagcctgaatatccgaatggtgttatcacgagttatggattatatctagatggtatattaatccacaattcctcagaactcagctatcgtgcttacggatttgctccttggagtttacattccttcagagtccaagcatgcacggccaaaggttgtgctctgggcccactggtggaaaatcgaactctagaagctcctcctgaaggaacagtaaatgtgtttgtcaaaacacagggatcccggaaagcccacgtgaggtgggaagcaccttttcgccctaatggactcttaacacactcagtccttttcactgggatattctatgtagacccagtaggtaataactacacccttctgaatgtcacaaaagtcatgtacagcggagaagagacaaacctttgggtgctcatcgatgggctggttccttttaccaactatactgtacaagtgaatatttcaaatagccaaggcagcttgataactgatcctataacaattgcaatgcctccaggagctccagatggcgtgctgcctcccaggctttcatctgccactccaaccagtcttcaggttgtctggtctacaccagctcgtaataacgctcctggctctcccagataccaactccagatgaggtctggcgactccacccatggatttctagagttattttccaatccttctgcatcgttaagctatgaagtgagtgatctccaaccgtacacagagtatatgtttcggttggttgcctccaatggatttggcagtgcacatagttcttggattccattcatgaccgcagaggacaaacctggacctgtagttcctccgattcttctggatgtgaagtcaagaatgatgttggtcacctggcagcatcctagaaaatccaatggggttattacccattataacatttatctacatggccgtctatacttgagaactcctggaaatgtcactaattgcacagtgatgcatttacacccatacactgcctataagtttcaggtagaagcctgcacttcaaaaggatgttccctttcaccagagtcccagactgtatggacactcccaggggcaccggaagggatcccaagtccagagctgttctctgatactccaacatctgtgattatatcttggcaaccccctacccaccccaatggcttggtggagaatttcacaattgagagaagagtcaaaggaaaggaagaagttactaccctggtgactctcccgaggagtcattccatgaggtttattgacaagacttctgctcttagcccatggacaaaatatgaatatcgggtactgatgagcactcttcatggaggcacaaacagcagtgcttgggtagaagttaccacaagaccctcacgacctgctggggtgcagccacctgtggtgacagtgctggaacccgatgcagtccaggtcacttggaaacccccactcatccagaacggagacatacttagctatgagattcacatgcctgaccctcacatcactttaaccaatgtgacttccgcagtgttaagtcaaaaagttactcatctgattcctttcactaattattctgtcaccattgttgcttgctcagggggtaatgggtaccttggagggtgcacagagagtttacctacctatgttaccactcaccccaccgtacctcagaatgttggcccattgtctgtgattccactaagtgaatcatatgttgtgatttcttggcaaccaccatccaagccaaatggacctaatttgagatatgagcttctgagacgtaaaatccagcagccacttgcatcaaatcccccagaagatttaaatcggtggcacaatatttattcaggaactcagtggctttatgaagataagggtcttagcaggtttacaacctatgaatatatgctcttcgtacacaacagtgtgggttttacaccgagccgagaagtgactgtgacaacgttagctggtcttccagagagaggagccaatctcactgcgagtgtccttaaccacacagccatcgacgtgaggtgggctaaaccaactgttcaagacctacaaggtgaagttgaatattacacacttttttggagttctgctacctcaaacgactctctaaaaatcttgccagatgtaaactctcatgtcattggccacctaaagccaaacacagagtattggatctttatctctgtcttcaatggagtccacagcatcaacagtgcaggacttcatgcaaccacttgcgatggggagcctcagggcatgcttcctccagaggttgtcatcatcaacagtacagctgtacgtgtcatctggacatctccttcaaacccaaatggtgttgtcactgagtattctatctatgtaaataataagctctacaagactggaatgaatgtgcctgggtcgtttattctgagagacctgtctcccttcactatctatgacattcaggttgaagtctgcacaatatatgcctgcgtgaaaagcaatggaacccaaattaccactgtggaagacactccaagtgatataccaacacccacaattcgtggcatcacttcaagatctcttcaaattgattgggtgtctccacggaagccaaatggcatcattcttggatatgatctcctatggaaaacatggtatccatgcgctaaaactcaaaagttagtgcaggatcagagtgatgagctctgcaaggcagtgaggtgtcaaaaacctgaatctatctgtggacacatttgctattcttctgaagctaaggtttgttgtaacggagtgctctataaccccaagcctggacatcgctgttgtgaagaaaagtatatcccgtttgttctgaattctactggagtttgttgtggtggccgaatacaggaggcacaaccaaatcatcagtgctgctctgggtattacgctagaattctaccaggtgaagtatgctgtccagatgaacagcacaatcgggtttctgttggcattggtgattcctgctgtggcagaatgccgtactccacctcaggaaaccagatttgctgtgctgggaggcttcatgatggccatggccagaagtgctgtggcagacagattgtgagcaacgatttagagtgttgtggtggagaagaaggagtggtgtacaatcgccttccaggtatgttctgttgtgggcaggattatgtgaatatgtcagataccatatgctgctcagcttccagtggagagtctaaagcacatattaaaaagaatgacccggtgccagtaaaatgctgtgagactgaacttattccaaagagccagaaatgctgtaatggagttggatataatcctttgaaatatgtttgctctgacaagatttcaactggaatgatgatgaaggaaaccaaagagtgcaggatcctctgcccagcatctatggaagccacagaacattgtggcaggtgtgacttcaactttaccagccacatttgcactgtgataagagggtctcacaattccacagggaaggcatcaattgaagaaatgtgttcatctgccgaagaaaccattcatacagggagtgtaaacacgtactcttacacagatgtgaacctcaagccctacatgacatatgagtacaggatttctgcctggaacagctatgggcgaggactcagcaaagctgtgagagccagaacaaaagaagatgtgcctcaaggagtgagtccccctacgtggaccaaaatagacaatcttgaagatacaattgtcttaaactggagaaaacctatacaatcaaatggtcctattatttactacatccttcttcgaaatggaattgaacgttttcggggaacatcactgagcttctctgataaagagggaattcaaccatttcaggaatattcatatcagctgaaagcttgcacggttgctggctgtgccaccagtagcaaggtagttgcagctactacccaaggagttccggagagcatcctgccaccaagcatcacagccctaagtgcagtggctctgcatctgagctggagtgtccctgagaaatcaaacggcgtcattaaagagtaccagatcaggcaggttgggaaaggtctcatccacactgacaccactgacaggagacagcatacggtcacaggtctccagccatacaccaactacagcttcactcttacagcttgtacatctgctgggtgcacttcaagcgagccttttctaggtcagacactgcaggcagctcctgaaggagtttgggtgacacctcgacacattatcatcaattctacaacagtggaattatattggagtctgccagaaaagcccaatggcctcgtttctcaatatcaattgagtcgtaatggaaacttgcttttcctgggtggcagtgaggagcagaatttcactgataaaaacctggagcccaatagcagatacacttacaagttagaagtcaaaactggaggtggcagcagtgctagtgatgattacattgttcaaacacctatgtcaacaccagaagaaatctatcctccatataatatcacagtaattgggccttattctatatttgtagcttggataccaccagggatcctcatccccgaaattcctgtggagtacaatgtcttactcaatgatggaagtgtaacacctctggccttctccgttggtcatcatcaatccacccttctggaaaatttgactccattcacacagtatgagataaggatacaagcatgtcaaaatggaagttgtggagttagcagtaggatgtttgtcaaaacacctgaagcagccccaatggatcttaattctcctgttcttaaggcactggggtcagcttgcatagagattaagtggatgccacctgaaaaaccaaatggaatcatcatcaactactttatttacagacgccctgctggcattgaagaggagtctgttttatttgtctggtcagaaggagcccttgaatttatggatgaaggagacaccctgaggcctttcacactctacgaatatcgggtcagagcctgtaactccaagggttcagtggagagtctgtggtcattaacacaaactctggaagctccacctcaagattttccagctccttgggctcaagccacgagtgctcattcagttctgttgaattggacaaagccagaatctcccaatggcattatctcccattaccgtgtggtctaccaggagagacccgacgatcctacatttaacagccctaccgtgcatgctttcacagtgaagggaacaagccatcaagcccacctgtacgggttagaaccattcacaacatatcgcattggtgttgtggctgcaaaccatgcaggagaaattttaagcccttggactctgattcaaaccttagaatcttccccaagtggactgagaaactttatagtagaacagaaagagaatggccgggcattgctactacagtggtcagaacctatgagaaccaatggtgtgattaagacatacaacatcttcagtgacgggttcctggagtactctggtttgaatcgtcagtttctcttccgccgcctggatcctttcactctctacacactgaccctggaggcctgcaccagagcaggttgtgcacactcggcgcctcagcctctgtggacagatgaagcccctccagactctcagctggctcctactgtccactctgtgaagtccaccagtgttgagctgagctggtctgagcctgttaacccaaatggaaaaataattcgctatgaagtgattcgcagatgcttcgagggaaaagcttggggaaatcagacaatccaggccgacgagaaaattgttttcacagaatataacactgaaaggaatacatttatgtataatgacacaggtttgcaaccatggacgcagtgtgaatataaaatctacacttggaattcagctgggcatacctgtagctcttggaatgtggtgaggacattgcaagcacctccagaaggtctctctccacctgtgatatcctatgtttctatgaatccccaaaaactgctgatttcctggatcccaccagaacagtctaatggtattatccagtcctataggcttcaaaggaatgaaatgctctatccttttagctttgatcctgtgactttcaattacactgatgaagagcttcttcctttttccacctatagctatgcactccaagcctgcacgagtggaggatgctccaccagcaaacccaccagcatcacaactctggaggctgctccatcagaagtcagccctccagatctttgggccgtcagtgccactcaaatgaatgtatgttggtcaccgcccacagtgcaaaatggaaagattactaaatatttagttagatatgataataaagagtcccttgctggccagggcctgtgcctgctggtttcccacctgcagccttactctcagtataacttctcccttgtagcctgcacgaatggaggttgcacagctagtgtgtcaaaatctgcctggacaatggaggccctgccagagaacatggactctccaacattgcaagtcacaggctcagaatcaatagaaatcacctggaaacctccaagaaacccaaatggccagatcagaagttatgaacttaggagggatggaaccattgtatatacaggcttggaaacacgctatcgtgattttactctcaccccaggtgtggagtatagctacacagtaactgccagcaacagccaagggggtattttgagtcctcttgtcaaagatcgaaccagcccctcagcaccctcagggatggaacctccaaaattgcaggccaggggtcctcaggagatcttagtgaactgggaccctccagtgagaacaaatggtgatatcatcaattataccctcttcatccgtgaactatttgaaagagaaactaaaatcatacacataaacacaactcataattcttttggtatgcagtcatatatagtaaaccagctgaagccatttcacaggtatgaaatacgaattcaagcgtgcaccaccctgggatgtgcatcaagtgactggacattcatacagacccctgagattgcacctttgatgcaaccccctccacatctggaggtacaaatggctccaggaggattccagccaactgtttctcttttgtggacaggaccgctgcagccaaatggaaaagttttgtattacgaattatacagaagacaaatagcaactcagcctagaaaatccaatccagtcctaatctataacggaagctcaacatcttttatagattccgaactattgcctttcacagagtatgagtatcaggtctgggcagtgaattctgcaggaaaagcccccagtagctggacatggtgcagaaccgggccagccccaccagaaggtctcagagcccccacgttccatgtgatctcttctacccaagcagtggtcaacatcagtgcccctgggaagcccaacgggatcgtcagtctctacaggctgttctccagcagcgcccatggggctgagacagtgctatccgaaggcatggccacccagcagactctccatggccttcaagccttcactaactactctattggagtagaggcctgcacctgcttcaactgttgcagcaaaggaccgacagctgaactgagaacccatcctgccccaccctcaggactgtcctctccacaaatcgggacgctggcctcaaggacggcctccttccggtggagtccccccatgttccccaatggtgtcattcacagctatgaactccaattccacgtggcttgccctcctgactcagccctcccctgtactcccagccaaatagaaacaaagtacacggggctggggcagaaagccagccttgggggtctccagccctacaccacatacaagctgagagtggtggcacacaacgaggtgggcagtacggcttccgagtggatcagtttcaccacccaaaaagaattgcctcagtaccgagccccattttcggtggacagcaatttgtctgtggtgtgtgtgaactggagtgacaccttcctcctgaacggccaactgaaggagtacgtgttaaccgacggagggcgacgcgtgtacagcggcttggacaccaccctctacataccgagaacggcggacaaaaccttctttttccaggtcatctgcacgactgacgaaggaagtgttaagacgccgttgatccaatatgatacctctactggacttggcttggtcctaacaactcctgggaaaaagaagggatcgcggagcaaaagcacagagttctacagcgagctgtggttcatagtgttaatggcgatgctgggcttgatcttgttggccatttttctgtccctgatactacaaagaaaaatccacaaagagccatatatcagagaaagacctcccttggtacctcttcagaagaggatgtctccattgaatgtttacccaccgggggaaaaccatatggggttagccgataccaaaattccccggtctgggacacctgtgagtatccgcagcaaccggagtgcatgtgtcctgcgcatcccgagtcaaaaccaaaccagcctaacctactcccagggttctcttcaccgcagcgtcagccagctcatggacattcaagacaagaaagtcttgatggacaactcactgtgggaagccatcatgggccacaacagtggactgtatgtggatgaagaggacctgatgaacgccatcaaggatttcagctcagtgactaaggaacgcaccacattcacagacacccacctgtaa

SEQ ID NO: 61 - DNA, 4029 bp, unidentified
  source 1..4029: mol_type = unassigned DNA
atgaagcgtattttctcactgctagaaaagacttggcttggcgcaccaatacagtttgcctggcaaaaaacatcaggaaactaccttgcagtaacaggagctgattatattgtgaaaatctttgatcgccatggtcaaaaaagaagtgaaattaacttacctggtaactgtgttgccatggattgggataaagatggagatgtcctagcagtgattgctgagaaatctagctgcatttatctttgggatgccaacacaaataagaccagccagttagacaatggcatgagggatcaaatgtctttccttctttggtcaaaagttggaagtttcctggctgttggaactgttaaaggaaatttgcttatttataatcatcagacatctcgaaagattcctgtccttggaaaacatactaagagaatcacttgtggatgttggaatgcagaaaatctgcttgctttaggtggtgaagataaaatgattacagttagtaatcaggaaggtgacacgataagacagacacaagtgagatcagagcctagcaacatgcagtttttcttgatgaagatggatgaccgaacctctgctgctgaaagcatgataagtgtggtgcttggcaagaaaactttgttttttttaaatctgaatgaaccagataacccagctgatcttgaatttcagcaggactttggcaacattgtctgctataattggtatggtgatggccgcatcatgattggtttttcatgtggacattttgtggtcatttctactcatactggagagcttggtcaagagatatttcaggctcgtaaccataaagataatctaaccagcattgcagtatcacagactcttaacaaagttgctacatgtggagataactgcattaaaatccaagacttggttgacttaaaagacatgtatgttatactcaacctggatgaggaaaataaaggattgggtaccttgtcctggactgatgatggccagttgctagcactctctacccaaaggggctcacttcatgttttcctgaccaagcttcccatacttggggatgcctgcagcacaaggattgcctatctcacctccctccttgaagtcaccgtagccaaccctgttgaaggagagctaccaatcacagtttctgttgatgtggaacccaactttgtggcagtaggtctttatcatctggctgtaggaatgaataatcgagcttggttttatgtccttggagaaaatgctgtgaaaaaattgaaagatatggagtatctgggaacagtagccagtatttgccttcattctgactatgctgctgcactttttgaaggcaaagtccagttacatttgatagaaagcgaaatcttggatgctcaagaagaacgtgagactcggcttttcccagcagtggatgataagtgccgtatcttatgccatgccttaactagtgatttcctcatctatggtacagatactggtgtcgttcagtatttctacattgaagactggcaattcgttaatgattatcgacatcctgtcagtgtgaaaaagatttttcccgacccaaatgggaccagattagttttcattgatgaaaaaagtgatggatttgtttactgtccagtcaatgacgctacctatgagattccagatttttcaccaaccattaaaggtgttctttgggaaaactggccaatggataaaggtgtatttattgcttatgatgatgataaggtgtacacttatgtctttcacaaggacactatacaaggagccaaggttattttggctggtagcaccaaagttccttttgctcataaacctttgctgctatataatggagagctgacctgccaaacacagagtggaaaagtaaacaacatctaccttagcacccatggctttctcagcaacttaaaagatacggggcctgacgaactgagaccaatgctggcacagaatttaatgctaaagaggttttctgatgcttgggaaatgtgcaggattctgaatgatgaggctgcctggaatgagttggccagagcttgtctacatcacatggaagtggagtttgcaatccgtgtttatcggagaattggaaatgttggcatagtgatgtccttggaacaaataaagggaatagaggactacaatcttttggcaggacaccttgccatgtttaccaacgattataacctggctcaggacttgtaccttgcatccagctgtcctattgctgccctggagatgagaagggatttacagcattgggacagtgctctacaactggcaaagcatttggccccagaccagataccttttatatcaaaagaatatgctattcagcttgaattcgcgggtgattatgtaaatgctttggctcattatgagaaaggaataacaggtgataataaggaacatgatgaagcttgtctggctggagtggcccagatgtccataagaatgggagacatacgtcgaggggttaaccaagccctcaagcatcccagcagggtccttaaaagagactgtggagccatattggagaatatgaagcaattttcagaagcggcccaactgtatgaaaaaggtctctactacgataaagcagcatctgtttacatccgctctaagaattgggcaaaagttggtgatcttctgccccacgtttcttctcctaagatccatttgcagtatgccaaagccaaggaagcagatggaagatacaaagaagctgttgtagcttatgaaaatgcaaaacagtggcaaagtgtaatccgcatctatctggatcacctcaataatcctgaaaaagctgtcaatattgttagagagacccagtctctggatggagccaaaatggtagccaggttttttctacagcttggtgactatgggtctgccatccagtttcttgtcatgtccaaatgcaacaatgaagctttcacactggctcagcaacacaacaaaatggaaatctatgcagatattattggttctgaagacactactaatgaagactatcaaagcattgccttatactttgaaggagaaaagagatatcttcaggctggaaaattcttcttgctgtgtggccaatattcacgagcacttaaacacttcctgaaatgcccaagctcggaagataatgtggcaatagaaatggcaattgaaactgttggtcaggccaaagatgaactgctgaccaatcagctgatagaccatctcctgggggagaacgatggcatgcctaaggatgccaagtacctgttccgcttgtacatggctctgaagcaataccgagaagctgcccagactgccatcatcattgccagagaagagcagtctgcaggcaactaccggaatgcacacgatgttctcttcagtatgtatgcagaactgaaatcccagaagatcaaaattccctccgagatggccaccaacctcatgattctgcacagctatatactagtaaagattcatgttaaaaatggagatcacatgaaaggggctcgcatgctcattcgggtggccaacaacatcagcaaatttccatcacacattgtacccatcctgacgtcaactgtgattgagtgtcacagggcaggcctgaagaactctgctttcagcttcgcagctatgttgatgaggcctgaataccgcagcaaaatagatgccaaatacaaaaagaagatcgagggaatggtcaggagacccgatatatctgagatagaagaggccacgactccatgtccattctgcaaatttcttctcccagagtgtgaactcctctgtcctggatgtaaaaacagtatcccatattgcattgcaacaggtcgacacatgttgaaagatgactggacggtgtgtccacattgtgacttccctgctctatactcagaattgaagatcatgctaaacactgaaagcacatgtcctatgtgttcagaaagattaaacgctgctcagctgaaaaagatttcagactgtacccagtacctgcgaacggaggaggaactgtga

SEQ ID NO: 62 - DNA, 1128 bp, unidentified
  source 1..1128: mol_type = unassigned DNA
atgaagctgacgcggaagatggttctgacccgggccaaggcctcggagctgcacagcgtgcgcaagctcaactgctggggcagccgcctcacagatatctccatttgccaggagatgcccagcctggaggtgatcacgctcagtgtcaacagcatctccaccctggagcctgtgagccggtgccagcgcctgagtgagctgtacctgcggaggaaccgcatccccagcctggctgagctcttctacctgaaggggctgccgcgtctgcgggtgctgtggctggccgagaacccgtgctgcggcaccagcccccaccgctaccgcatgaccgtgctgcgcaccctgccgcgcctacagaagctggacaaccaggctgtgacggaggaggagctgtcccgtgcactgagtgagggagaggagatcactgcggccccagagagagagggcacaggccacggcggccccaagctatgctgcacactgagctccctcagctccgctgctgagactggccgggacccgctggacagcgaggaggaggcaaccggcgcccaggatgaacgtggcctgaagccgccttcccggggccagtttccttccctctcagccagggatgcctcgagcagccacaggggcagggtgagtggcgggccgctaggggccgcggctgcctctgcccactgcacccactgcacagaaaccgtggggagggagcatggagcctcacagggccccgtggggagggagcatggagcctcacagggccttgaagagctgtgccccagggggagctgcgtgtgcgggtctgtgaatgcgcacacacgtgtaacacgtgccccgcacggagccgtcctggcccctcagcctctcctgctgtcctggtctgtggaatgtgggcccgggccctgctgggctgagggcaacaggagtcacgtggaagaggtgccacacacgcgtccacaggcggggctcctctgctcagattctccgagtgtgccgaacgtcctgactgccatcctgctgctgctgcgggagctggatgcagaggggctggaggccgtgcagcagactgtgggcagccggctgcaggccctgcgtggggaagaggtgcaggagcacgccgagtga

SEQ ID NO: 63 - DNA, 608 bp, Homo sapiens
  source 1..608: mol_type = genomic DNA
gggctcagaccttagagacgggttctcgacgatgtgcagcgcataacgaggaaaatactgaccggagggggccggtcgtagggctggggagcaggaggagggcaaaggatagagagggggtagatagggagtagatagggggcggcagcctcggcctcagtaagcaggggctcagggagcagatgtctgagactgcaccacgtccgctattttgaggtttggcttctcttactgccacgtggtgcagagggaaggcgccatttgatgcgtccaaatcccgagtgggaagagaagatacggatgagggagatggatgtgtctgtggtgactcttgggcacttgtgccgttcaatcctgtcactctcaagctgaagaacccaaagcaaacgccaggtttacaatacctgcactatcgctcattagtgggagggtcaggcgccacccaactctgggcttttattggtcatgtcaagagaaaagcgagagggacaactgggagcctccttctgattggtggattcctgctagccctcaagtcaatcagagtagaatgtaccgccctccttcccactctggcccccgcgggagacatgttaggtagcgggacg

SEQ ID NO: 64 - DNA, 457 bp, Homo sapiens
  source 1..457: mol_type = genomic DNA
ctctctcattctttacaaattcagcgaaaaaaattttttcaaaaattacaggttgcagatgaaactatataggctgcagcacaaataattcttcataggtgctgattactaataaagcaacaggtactggaaggattcattatctaaatgggtcaagccaatctagggaagattatcctctaatctggtggctgcatttacaacattccacaaaaagccgattaaagggagaggatgctattttaggctcttggaaaaataatgtctatggcctaggttattttgtatactaaatattttctcagtggagacaattaaagattaaattagaattggaggtcatttactagccattcatctgttttaggttaaacactgtgagatcagtttatctgaaaagtttcagctagcgttaacagtattaagctgaaaacagaatggatccagtgcaaaatca

SEQ ID NO: 65 - DNA, 460 bp, Homo sapiens
  source 1..460: mol_type = genomic DNA
ccaaccttgtcactcttctgagaccgccacccagtaacacgccctgctttcctccgccgccgccgtctcctccatcaactgtctccttctaacatgttattgcagttaataaaaaagcgttcttttcttcacaaaagcagttgtttcattgtataatgtgagaagtctgcgttcaggcggttagacgcaaaggagtggaataagtgccctgttcagcctgccaaccttcggaaccagccaacagactcggtactgactgccatgatccagtaagcacccgactcgggtctctggcagcctgagtggattttgcgtgacttttggtccctggcggctcctgtagcgtccccagttacggcgcccatagcaaccggctccctagctaggcgcccccgggttgccaggggccgcaccagctttcccgtcccgggccagcgcaggcgctcaggcctcggaggcg

SEQ ID NO: 66 - DNA, 1068 bp, Homo sapiens
  source 1..1068: mol_type = genomic DNA
gggctcagaccttagagacgggttctcgacgatgtgcagcgcataacgaggaaaatactgaccggagggggccggtcgtagggctggggagcaggaggagggcaaaggatagagagggggtagatagggagtagatagggggcggcagcctcggcctcagtaagcaggggctcagggagcagatgtctgagactgcaccacgtccgctattttgaggtttggcttctcttactgccacgtggtgcagagggaaggcgccatttgatgcgtccaaatcccgagtgggaagagaagatacggatgagggagatggatgtgtctgtggtgactcttgggcacttgtgccgttcaatcctgtcactctcaagctgaagaacccaaagcaaacgccaggtttacaatacctgcactatcgctcattagtgggagggtcaggcgccacccaactctgggcttttattggtcatgtcaagagaaaagcgagagggacaactgggagcctccttctgattggtggattcctgctagccctcaagtcaatcagagtagaatgtaccgccctccttcccactctggcccccgcgggagacatgttaggtagcgggacgccaaccttgtcactcttctgagaccgccacccagtaacacgccctgctttcctccgccgccgccgtctcctccatcaactgtctccttctaacatgttattgcagttaataaaaaagcgttcttttcttcacaaaagcagttgtttcattgtataatgtgagaagtctgcgttcaggcggttagacgcaaaggagtggaataagtgccctgttcagcctgccaaccttcggaaccagccaacagactcggtactgactgccatgatccagtaagcacccgactcgggtctctggcagcctgagtggattttgcgtgacttttggtccctggcggctcctgtagcgtccccagttacggcgcccatagcaaccggctccctagctaggcgcccccgggttgccaggggccgcaccagctttcccgtcccgggccagcgcaggcgctcaggcctcggaggcg

SEQ ID NO: 67 - DNA, 917 bp, Homo sapiens
  source 1..917: mol_type = genomic DNA
ctctctcattctttacaaattcagcgaaaaaaattttttcaaaaattacaggttgcagatgaaactatataggctgcagcacaaataattcttcataggtgctgattactaataaagcaacaggtactggaaggattcattatctaaatgggtcaagccaatctagggaagattatcctctaatctggtggctgcatttacaacattccacaaaaagccgattaaagggagaggatgctattttaggctcttggaaaaataatgtctatggcctaggttattttgtatactaaatattttctcagtggagacaattaaagattaaattagaattggaggtcatttactagccattcatctgttttaggttaaacactgtgagatcagtttatctgaaaagtttcagctagcgttaacagtattaagctgaaaacagaatggatccagtgcaaaatcaccaaccttgtcactcttctgagaccgccacccagtaacacgccctgctttcctccgccgccgccgtctcctccatcaactgtctccttctaacatgttattgcagttaataaaaaagcgttcttttcttcacaaaagcagttgtttcattgtataatgtgagaagtctgcgttcaggcggttagacgcaaaggagtggaataagtgccctgttcagcctgccaaccttcggaaccagccaacagactcggtactgactgccatgatccagtaagcacccgactcgggtctctggcagcctgagtggattttgcgtgacttttggtccctggcggctcctgtagcgtccccagttacggcgcccatagcaaccggctccctagctaggcgcccccgggttgccaggggccgcaccagctttcccgtcccgggccagcgcaggcgctcaggcctcggaggcg

(19) United States
(12) Patent Application Publication
(10) Pub. No.: US 2026/0000786 A1
(43) Pub. Date: Jan. 1, 2026

(54) GENE THERAPY FOR FAM161A-ASSOCIATED RETINOPATHIES AND OTHER CILIOPATHIES

(71) Applicants: FONDATION ASILE DES AVEUGLES, Lausanne (CH); HADASIT MEDICAL RESEARCH SERVICES AND DEVELOPMENT LTD, Jerusalem (IL)

(72) Inventors: Yvan ARSENIJEVIC, Lausanne (CH); Corinne KOSTIC, Lausanne (CH); Ning CHANG, Lausanne (CH); Sharon DROR, Jerusalem (IL); Banin EYAL, Jerusalem (IL); Carlo RIVOLTA, Epalinges (CH)

(21) Appl. No.: 18/881,384

(22) PCT Filed: Jul. 12, 2023
(86) PCT No.: PCT/EP2023/069304

§ 371 Date: Jan. 6, 2025

Related U.S. Application Data
(60) Provisional application No. 63/368,362, filed on Jul. 14, 2022.

(51) Int. Cl. A61K 48/00; A61K 38/17; A61P 27/02; C12N 15/86

(52) U.S. Cl. CPC A61K 48/005; A61K 38/1709; A61P 27/02; C12N 15/86; C12N 2750/14122; C12N 2750/14143; C12N 2830/48

(57) ABSTRACT

The present invention is directed vectors comprising a FCBR1-F0.4 promotor or an IRBP-GRK1 promotor and a nucleic acid sequence encoding a ciliary protein, or fragment or variant thereof. In particular, the invention relates to a method of treatment and prevention of retinal ciliopathies, such as retinitis pigmentosa 28.

### FIELD OF THE INVENTION

The present invention relates to the field of production of vectors encoding ciliary proteins and to methods of treatment and/or prevention of retinal ciliopathies. In particular, the present invention relates to a method of treatment and/or prevention of FAM161A-associated retinopathies, such as retinitis pigmentosa 28 induced by the deficiency of FAM161A protein.

### BACKGROUND OF THE INVENTION

Retinitis pigmentosa (RP) is a group of genetic disorders involving progressive degeneration of the retina that affects the retina's ability to respond to light, causing a slow loss of vision. RP is genetically and clinically heterogeneous, and is characterized by night blindness, progressive degeneration of photoreceptors leading to gradual loss of peripheral and then central vision, and eventually often leads to blindness.

At present, mutations in more than 60 genes were linked to non-syndromic RP, 41 of them were reported to cause autosomal recessive (AR) disease, including FAM161A, which was initially identified in Israeli, Palestinian, Indian, North American, and European patients (Beryozkin et al., 2020, Scientific Reports, 10:15156). In particular, retinitis pigmentosa-28 (RP28) is an autosomal recessive retinitis pigmentosa (arRP) caused by pathogenic variants in the gene FAM161A (Langmann et al., 2010, Am J Hum Genet. 87 (3): 376-381; Bandah-Rozenfeld et al., 2010, Am J Hum Genet. 87 (3): 382-91). Retinitis pigmentosa-28 (RP28) is characterized by a slow retina degenerative process, but with visual impairment detected early in life, and a rapid decline of retina activity from the fourth-fifth decade (Beryozkin et al., 2020, supra). FAM161A protein localizes at the photoreceptor connecting cilium in human, mouse, and rat and it is also present at the ciliary basal body in ciliated mammalian cells (Di Gioia et al., 2012, Hum Mol Genet. 21 (23): 5174-84; Di Gioia et al., 2015, Hum Mol Genet. 24 (12): 3359-71). To study pathological mechanism of RP28 disease progression and assist in designing therapeutic strategies, a knockout (KO) mouse model was generated Fam161a^{tm1b/tm1b}), lacking the major exon #3 which was replaced by a construct that include LacZ under the expression of the Fam161a promoter) (Beryozkin et al., 2021, Scientific Reports 11:2030). So far it was shown that the absence of FAM161A alters the maintenance of the cilium structure leading to progressive photoreceptor outer segment alteration, wherein the outer segments are crucial for photoreceptor function by converting the light stimulus into an electrical signal (Karlstetter et al., 2014 Hum. Mol. Gen. 23 (19), 5197-5210; Mercey et al., 2022, PLOS Biol e3001649). Presently, there is no treatment for RP28 disease.

Therefore, there is a need for a method of prevention and/or treatment of retinitis pigmentosa-28.

### SUMMARY OF THE INVENTION

The present invention is based on the surprising finding that vectors comprising human FAM161A gene, both long (hFAM161A-L) or short (hFAM161A-S) isoform, and comprising human FCBR1-F0.4 promotor (FCBR, Crx binding region; F0.4, FAM161A core promoter) are particularly suitable to express human FAM161A protein, both long (hFAM161A-L) or short (hFAM161A-S) isoform in the photoreceptor's connecting cilium (Table 1). Together with FAM161A protein expression restoration, improve visual function is observed by ERG recordings. The FCBR2-F0.4 promotor with human short (hFAM161A-S) isoform gave similar ERG improvement at 2 month-post-injection (Table 1), but the expression is not exclusively addressed in the connecting cilium and is present in the whole photoreceptor. On the other hand, the presence of juxtaposed FCBR2-FCBR1 sequences with the core promoter combined with the mouse ML1 (mouse long 1; mFam161a-L1) isoform increased the degenerative process. The vectors of the invention are shown to provide an adequate level of protein expression in a FAM161A knockout (KO) mouse model (Fam161a^{tm1b/tm1b} referred herein as Fam161^{-/-}) that allows to restore photoreceptor's anatomy and photoreceptor's function (Table 1 and FIGS. 5-7, 11-13). Therefore, the vectors of the invention are proposed for use in therapy of disorders associated with mutations in FAM161A gene, such as FAM161A-associated retinopathies, in particular for therapy of retinitis pigmentosa-28 (RP28).

Since FAM161A is an example of a ciliary protein, that are proteins found in photoreceptors' cilia connecting the inner and outer segments, the vectors comprising in particular FCBR1-F0.4 promotor (among others) and transgene encoding any ciliary protein are proposed for use in therapy of associated ciliopathies.

Success and efficient protection of retina integrity and function preservation can be achieved with IRBP-GRK1 promotor driving expression of at least the mouse L isoform (Table 1, FIGS. 5-7).

However, IRBP-GRK1 promotor combined with the HL or HS isoform did not improve the maintenance of mouse retinal activity and the co-administration of both HL+HS isoforms with IRBP-GRK1 vectors did not allow the mouse retina activity after 3 months post-injection (m.p.i). In addition, this promotor is more efficient when driving the expressions of the mouse ML isoform (FIG. 6) than the human ones (HS or HL, FIG. 9).

One aspect of the invention provides a vector comprising a FCBR1-F0.4 promotor and a nucleic acid sequence encoding a ciliary protein, or a fragment or a variant thereof, such as wherein FCBR1-F0.4 promotor is of SEQ ID NO: 10 or SEQ ID NO: 66.

Another aspect of the invention provides a vector comprising an IRBP-GRK1 promotor and a nucleic acid sequence encoding a ciliary protein, or a fragment or a variant thereof, such as wherein IRBP-GRK1 promotor is of SEQ ID NO: 14

Another aspect of the invention provides a vector as described herein, wherein a nucleic acid sequence encoding a ciliary protein is selected from a long isoform of human FAM161A gene of SEQ ID NO: 1 and a short isoform of human FAM161A gene of SEQ ID NO: 2, or a fragment, or a variant thereof.

Another aspect of the invention provides a vector as described herein, wherein a nucleic acid sequence encoding a ciliary protein is selected from POC5 gene of SEQ ID NO: 29, NPHP4 gene of SEQ ID NO: 30, ARL6 gene of SEQ ID NO: 31, BBS1 gene of SEQ ID NO: 32, BBS2 gene of SEQ ID NO: 33, BBS9 gene of SEQ ID NO: 34, PCARE gene of SEQ ID NO: 35, CFAP418 gene of SEQ ID NO: 36, CEP164 gene of SEQ ID NO: 37 (CEP164 isoform 1) or of SEQ ID NO: 38 (CEP164 isoform 2), CEP290 gene of SEQ ID NO: 39, CLRN1 gene of SEQ ID NO: 40, IFT140 gene of SEQ ID NO: 41, IFT172 gene of SEQ ID NO: 42, IQCB1 gene of SEQ ID NO: 43, KIZ gene of SEQ ID NO: 44, LCA5 gene of SEQ ID NO: 45, MAK gene of SEQ ID NO: 46, NEK2 gene of SEQ ID NO: 47, OFD1 gene of SEQ ID NO: 48, OFD1 gene of SEQ ID NO: 49, RP1 gene of SEQ ID NO: 50, RP1L1 gene of SEQ ID NO: 51, RP2 gene of SEQ ID NO: 52, RPGR gene of SEQ ID NO: 53 (RPGR isoform C) or SEQ ID NO: 54 (RPGR isoform J), RPGRIP1 gene of SEQ ID NO: 55 (RPGRIP1 isoform 1) or SEQ ID NO: 56 (RPGRIP1 isoform 3), SPATA7 gene of SEQ ID NO: 57, TOPORS gene of SEQ ID NO: 58, TTC8 gene of SEQ ID NO: 59, USH2A gene of SEQ ID NO: 60, WDR19 gene of SEQ ID NO: 61, CFAP410 gene of SEQ ID NO:62, or a fragment or a variant thereof.

Another aspect of the invention provides a vector as described herein, further comprising a WPRE (woodchuck hepatitis virus post-transcriptional regulatory element) post-transcriptional regulatory element of SEQ ID NO: 15, or a fragment or a variant thereof.

Another aspect of the invention provides a vector as described herein that is a recombinant AAV vector that has an AAV2/8 capsid protein or an AAV2/5 capsid protein.

Another aspect of the invention relates to a pharmaceutical composition comprising at least one vector according to the invention or a combination thereof and at least one pharmaceutically acceptable vehicle.

Another aspect of the invention relates to a vector as described herein for use as a medicament.

Another aspect of the invention relates to a vector according to the invention or a pharmaceutical composition thereof for use in the modification, prevention, delaying, arresting progression, or ameliorating of vision loss associated with a retinal ciliopathy, such as FAM161A-associated retinopathies.

Another aspect of the invention relates to a vector according to the invention or a pharmaceutical composition thereof for use in the treatment of a retinal ciliopathy, such as FAM161A-associated retinopathies, preferably retinitis pigmentosa 28.

Another aspect of the invention relates to a vector according to the invention or a pharmaceutical composition thereof for use in the improvement or maintenance of photoreceptor structure.

Another aspect of the invention relates to a vector according to the invention or a pharmaceutical composition thereof for use in the induction of photoreceptor cytoskeleton stabilization.

### DESCRIPTION OF THE FIGURES

FIG. 1 shows several plasmid constructs designed as described in Example 1.

FIG. 2 shows in vitro FAM161A protein expression as described in Example 2. A) shows human FAM161A protein isoforms (long and short, HL and HS respectively) detected by Western blotting in the 661W cells. B) shows mouse FAM161A protein (mFAM161A) isoforms (long, short and short 1, ML, MS and MS1 respectively-only ML, MS, and MS1 are shown in the figure) detected by Western blotting in the HEK293T cells. C-G) shows immunocytochemistry of hFAM161A or mFAM161A isoforms and acetylated tubulin (Ac-tub) (C) HL; D) HS; E) ML; F) MS; G) MS1). hFAM161A or mFAM161A isoforms target different cellular compartments and induce tubulin stabilization. H) shows quantification of stabilized acetylated tubulin percentage of transfected cells.

FIG. 3 shows transgene plasmids used to generate the following vectors as referred to in Examples 3 and 5: A) AAV2/8-IRBP-GRK1-hFAM161A-L-WPRE, B) AAV2/8-IRBP-GRK1-hFAM161A-S-WPRE, C) AAV2/8-FCBR1-F0.4-hFAM161A-L-WPRE, D) AAV2/8-FCBR1-F0.4-hFAM161A-S-WPRE.

FIG. 4 shows FAF images (fundus autofluorescence) as described in Example 3. In panel A (A.1-A.5) serial FAF images of non-treated Fam161^{-/-} retinas showing narrowing of blood vessels and formation of patchy autofluorescent spots indicating widespread retinal degeneration, at 1, 3, 4.5, 6 or 8 months (m) old. Following gene augmentation therapy (injection of AAV2/8-IRBP-GRK1-mFam161a^{-/-} L vector at the age of 3-4 weeks) shown in panel B (B.1-B.4), a significant decrease in the formation of autofluorescent spots can be seen in the treated areas, at 3, 4.5, 6 or 8 months old (2, 3.5, 5 and 7 months post-injection respectively) (left side of each image) as compared to untreated retinal regions (right side).

FIG. 5 shows OCT images as described in Example 3. OCT images of an AAV2/8-IRBP-GRK1-mFam161a^{-/-} L vector treated eye at 3, 6, and 8 months of age showing that photoreceptors in the treated regions survive for at least 8 months following gene therapy. The vector was injected at P21-29 when the cilium is already altered. Note the rescued ONL thickness (arrows) in comparison to the contralateral side.

FIG. 6 shows dark-adapted (A-E) ERG recordings at 3-, 4.5-, 6- and 8-months post-injection of AAV2/8-IRBP-GRK1-mFam161a^{-/-} L treated compared to non-treated eyes, as described in Examples 3. The results show that functional rescue persists for at least 8 months following gene therapy. stimulation (* p-value<0.05, #p-value<0.01). (F) The visual acuity was markedly improved in the ML treated group in comparison to the control non-treated eye (panel F) (* p-value<0.05, *** p-value<0.01).

FIG. 7 shows histological analysis of a representative eye as described in Example 3. Eye (8 months after vector injection) that underwent AAV2/8-IRBP-GRK1-mFam161a^{-/-} L gene therapy at P21-29 showing clear-cut preservation of the ONL in the treated area compared to the non-treated area. (A) Quantification of ONL thickness. (B) Section of a Fam161a^{-/-} retina treated with the vector on its left side.

FIG. 8 shows ERG recording 1-month (A), 2-months (B) and 3-month (C) post injection (p.i.) of the 10¹⁰ gc/ul AAV2/8-IRBP-GRK1-hFAM161A-L-WPRE ((1); AAV-IG-HL-W) or AAV2/8-IRBP-GRK1-hFAM161A-S-WPRE ((2); AAV-IG-HS-W) or control (AAV-IG-GFP-W) vectors injected in Fam161^{-/-} mouse as compared to an untreated eye, as described in Example 4.

FIG. 9 shows ERG recording 1-month (A), 2-months (B) and 3-month (C) post injection (p.i.) of the 0.5×10¹⁰ gc/ul AAV2/8-IRBP-GRK1-hFAM161A-L-WPRE ((1); AAV-IG-HL-W) mixed with 0.5×10¹⁰ gc/ul AAV2/8-IRBP-GRK1-hFAM161A-S-WPRE ((2); AAV-IG-HS-W) or control (AAV-IG-GFP-W, 10¹⁰ gc/ul) vectors injected in Fam161^{-/-} mouse, as described in Example 4.

FIG. 10 shows localization of FAM161A protein in the wild type (WT, A) and the Fam161a^{-/-} mice retina injected with AAV2/8-IRBP-GRK1-hFAM161A-S-WPRE ((2); AAV-IG-HS-W, C) or with AAV2/8-IRBP-GRK1-hFAM161A-L-WPRE ((1); AAV-IG-HS-W, E) (B): untreated the Fam161a^{-/-} retina. In (A), the arrows points to the normal FAM161A expression in the WT retina corresponding to the connecting cilium (CC). In (C), the arrows indicate ectopic expression of the FAM161A protein. In (D and E), the arrows indicate the retina region protected by the treatment. (F) shows the quantification of the retina thickness after AAV treatments. For information, WT (wild type mice) retina thickness is around 60 μM (not shown). ONL: outer nuclear layer (corresponding to photoreceptor cells).

FIG. 11 shows ERG recordings 3-month post injection of the AAV2/8 FCBR1-F0.4-hFAM161A-L-WPRE (10) (A), FCBR1-F0.4-hFAM161A-S-WPRE (11) (B) and AAV2/8 FCBR1-F0.4-hFAM161A-S-WPRE+-F0.4-hFAM161A-L-WPRE (10+11) (C) vectors injected in Fam161a^{-/-} mouse, as described in Example 6. The six best responses were analyzed for each group.

FIG. 12 shows FAM161A immunohistochemistry in Fam161a^{-/-} retina injected with AAV2/8-FCBR1-F0.4-hFAM161A-S-WPRE (11) (A) or AAV2/8-FCBR1-F0.4-hFAM161A-L-WPRE (10) (B), or both (C), or with AAV2/8-FCBR2-F0.4-hFAM161A-S-WPRE (7) (E). (D) shows the untreated area of the above HL tread retina. In (A) and (E), the white arrows indicate the ectopic expression of FAM161A, whereas these white arrows indicate the correct expression of FAM161A in the connecting cilium in (B and C). (F) shows the rescued retina thickness from the optic nerve (point 0) to the periphery in mice treated with the above-mentioned vectors. Note that the combination of HS+HL gave the best rescue of the retina thickness.

FIG. 13 shows ERG recordings 7 month post injection of the AAV2/8-FCBR1-F0.4-mouse-Fam161a-L-WPRE (A) and retina histology (B). During the treatment, mice received either one single injection (dorsal retina) or a double injection (dorsal and ventral). Arrows in (B) indicate the rescued ONL in the treated area. This representative mouse received one single injection.

### DETAILED DESCRIPTION

The term “photoreceptor” refers herein to a specialized type of sensory neuron cell found in the retina that is capable of visual phototransduction, i.e., rods and cones. There are three known types of photoreceptor cells in mammalian eyes: rods (contribute to sight), cones (contribute to sight), and intrinsically photosensitive retinal ganglion cells (do not contribute to sight directly). Rods primarily contribute to night-time vision (scotopic conditions) whereas cones primarily contribute to day-time vision (photopic conditions), but the chemical process in each that supports phototransduction is similar. The two photoreceptor cells i.e., rods and cones are found on the outermost layer of the retina and they both have the same basic structure. Closest to the visual field (and farthest from the brain) is the axon terminal, which releases a neurotransmitter (glutamate) to bipolar cells. Farther back is the cell body, which contains the cell's organelles. Farther back still is the inner segment (IS), a specialized part of the cell full of mitochondria (provide ATP (energy) for the sodium-potassium pump). Finally, closest to the brain (and farthest from the field of view) is the outer segment (OS), the part of the photoreceptor that absorbs light to convert it in an electrical signal. Outer segments are actually modified cilia that contain disks filled with opsin, the molecule that absorbs photons, as well as voltage-gated sodium channels.

The term “connecting cilium” or “CC” (or cilia in plural) or “a transition zone” refers herein to a particular structure of rods and cones that is located between their inner and outer segments (IS and OS). The CC is a defined structure of the cilium where the 9 Tubulin filaments are assembled by other proteins, such as FAM161A, to form a tube to address opsins at the correct location. The CC is thus involved in the transport of molecules across these two compartments. The “FAM161A” refers herein to a human gene encoding FAM161A protein. FAM161A gene produces two different mRNA isoforms in the retina. “FAM161A” is a human protein found in the CC of photoreceptors and is thus a ciliary protein (Di Gioia et al., 2012, supra; Karlstetter et al., 2014, supra). In addition, FAM161A is also a component of the Golgi-centrosomal network (Di Gioia et al., 2015, supra). The function of FAM161A protein is not completely established. FAM161A is a microtubule-associated ciliary protein presumably involved in maintaining microtubule stability (Karlstetter et al., 2014, supra; Mercey et al., 2021, supra). The “Fam161a” refers herein to a mouse gene encoding mouse “Fam161a” protein.

The term “retinitis pigmentosa-28” or “RP28” refers to an autosomal recessive retinitis pigmentosa (arRP) caused by pathogenic variants in the gene FAM161A.

The term “FAM161A-associated retinopathy” or “FAM161A-associated RP” refers to retinal ciliopathy (retinopathy) caused by pathogenic variants in the gene FAM161A. One example is RP-28. It is understood that some patients with mutations in the FAM161A gene will not have a phenotype of the retinitis pigmentosa 28, but another phenotype.

The term “retinal ciliopathies” refers to a group of disorders associated with pathophysiology of the photoreceptor cilia. The retinal ciliopathies include, but are not limited to retinitis pigmentosa such as retinitis pigmentosa-28, FAM161A-associated RP, RP, BBS (Bardet-Biedl syndrome), SLS (Senior-Løken syndrome), JBS (Joubert syndrome), MKS (Meckel-Gruber syndrome), USH (Usher syndrome), JATD (Jeune asphyxiating thoracic dystrophy), MZSDS (Mainzer-Saldino syndrome), OFD (oral-facial-digital syndrome), OMD (occult macular dystrophy), CRD (cone-rod dystrophy), LCA (Leber congenital amaurosis), CED (cranioectodermal dysplasia, also known as Sensenbrenner syndrome) (Bujakowska et al., 2017, Cold Spring Harb Perspect Biol. 10: a028274).

The term “adeno-associated virus” or “AAV” refers to a virus that is essentially composed of a protein shell surrounding and protecting a small, single-stranded DNA genome of approximately 4.8 kilobases (kb). Its single-stranded genome contains three genes, Rep (Replication), Cap (Capsid), and aap (Assembly) and these coding sequences are flanked by inverted terminal repeats (ITRs) that are required for genome replication and packaging. “Recombinant AAV” or “rAAV” refers to a virus that lacks viral DNA and is essentially a protein-based nanoparticle able to traverse the cell membrane, where it can ultimately traffic and deliver its DNA cargo into the nucleus of a cell. The rAAV also termed “AAV vector” is engineered to not replicate. In the absence of Rep proteins, ITR-flanked transgenes encoded within rAAV can form circular concatemers that persist as episomes in the nucleus of transduced cells. Because recombinant episomal DNA does not integrate into host genomes (or at very low efficacy), it will eventually be diluted over time as the cell undergoes repeated rounds of replication. This will eventually result in the loss of the transgene and transgene expression, with the rate of transgene loss dependent on the turnover rate of the transduced cell. These characteristics make rAAV a good candidate for certain gene therapy applications, especially for post-mitotic cells such as neurons, including photoreceptors. Typically, in rAAV design the sequences placed between the ITRs will typically include a mammalian promoter, gene of interest (i.e., transgene), and a terminator. Typically, a nucleic acid sequence encoding a gene of interest is a cDNA (complementary DNA) or shRNA (short hairpin RNA or small hairpin RNA). In addition, terminator/polyadenylation signal elements, the inclusion of post-transcriptional regulator elements and messenger RNA (mRNA) stability elements, and the presence of microRNA (miRNA) target sequence in the gene cassette can be considered (referred to as “cis-regulatory elements”). All the elements between the ITRs may be referred to as “rAAV expression cassette” or “transgene (expression) cassette”. “Post-transcriptional regulatory elements” refer to nucleotide sequences located upstream (5′ noncoding sequences), within, or downstream (3′ non-coding sequences) of a coding sequence, and which influence RNA processing or stability, or translation of the associated coding sequence. Regulatory elements may include, but are not limited to translation leader sequences, introns, and polyadenylation recognition sequences. Regulatory elements present on a recombinant DNA construct that is introduced into a cell can be endogenous to the cell, or they can be heterologous with respect to the cell. The terms “regulatory element” and “regulatory sequence” are used interchangeably herein.

Recombinant AAV are based on AAV selected from natural serotypes (AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9)) or on hybrid AAV vectors that have been engineered using genome of AAV serotype 2 and capsid protein of AAV serotypes 1-9 (e.g., AAV2/1, AAV2/2, AAV2/4, AAV2/5, AAV2/6, AAV2/7, AAV2/8 and AAV2/9).

The term “vector,” as used herein, is intended to refer to a nucleic acid molecule capable of transporting a nucleic acid sequence encoding a ciliary protein to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as “recombinant expression vectors” (or simply, “recombinant vectors”).

By the term “protein fragment” or “protein functional fragment”, it is meant any fragment that retains the function of the full-length protein. Similarly, by the term “protein variant” or “protein functional variant”, it is meant any protein variant that retains the function of the full-length protein. Functional protein fragments or variants of ciliary proteins or FAM161A may be determined by one of skill in the art according to known methods.

By the term “gene fragment” or “gene functional fragment”, it is meant any fragment that retains the function of the full-length gene, although not necessarily at the same level of expression or activity. Similarly, by the term “gene variant” or “gene functional variant”, it is meant any variant that retains the function of the full-length gene, although not necessarily at the same level of expression or activity. Functional fragments or variants of sequences encoding ciliary proteins or FAM161A may be determined by one of skill in the art according to known methods.

### Ciliary Proteins

The term “ciliary protein” or “protein of cilium” refers to proteins found in the specialised photoreceptor cilia and includes FAM161A, POC5, NPHP4, ARL6, BBS1, BBS2, BBS9, PCARE, CFAP418, CEP164, CEP290, CLRN1, IFT140, IFT172, IQCB1, KIZ, LAC5, MAK, NEK2, OFD1, RAB28, RP1, RP1L1, RP2, RPGR, RPGRIP1, SPATA7, TOPORS, TTC8, USH2A, WDR19, CFAP410 (Bujakowska et al., 2017, Cold Spring Harb Perspect Biol. 10: a028274).

In one embodiment the ciliary proteins are selected from the list comprising:
FAM161A; wherein a sequence encoding a long isoform of human FAM161A gene is such as of SEQ ID NO: 1 and a sequence encoding a short isoform of human FAM161A gene is such as of SEQ ID NO: 2;
POC5 (protein name: Centrosomal protein POC5; gene: proteome of centriole 5 (POC5); alias C5orf37), wherein a sequence encoding POC5 is such as of SEQ ID NO: 29,
NPHP4 (protein name: nephrocystin 4; gene: NPHP4; alias POC10; SLSN4; KIAA067), wherein a sequence encoding NPHP4 is such as of SEQ ID NO: 30;
ARL6 (protein name: ADP ribosylation factor like GTPase 6; gene: ARL6; alias BBS3, RP55), wherein a sequence encoding ARL6 is such as of SEQ ID NO: 31;
BBS1 (protein name: Bardet-Biedl syndrome 1 protein; gene: BBS1; alias BBS2L2), wherein a sequence encoding BBS1 is such as of SEQ ID NO: 32;
BBS2 (protein name: Bardet-Biedl syndrome 2; gene: BBS2; alias BBS, RP74), wherein a sequence encoding BBS2 is such as of SEQ ID NO: 33;
BBS9 (protein name: Bardet-Biedl syndrome 9; gene: BBS9; alias B1, C18, D1, PTHB1), wherein a sequence encoding BBS9 is such as of SEQ ID NO: 34;
PCARE (protein name: photoreceptor cilium actin regulator; gene: PCARE; alias RP54, C2orf71), wherein a sequence encoding PCARE is such as of SEQ ID NO: 35;
CFAP418 (protein name: cilia and flagella associated protein 418; gene: CFAP418, alias RP64, BBS21, MOT25, CORD16, FAP418, C8orf37, smalltalk), wherein a sequence encoding CFAP418 is such as of SEQ ID NO: 36;
CEP164 (protein name: centrosomal protein 164; gene: CEP164, alias NPHP15), wherein a sequence encoding CEP164 is such as of SEQ ID NO: 37 (CEP164 isoform 1) or of SEQ ID NO: 38 (CEP164 isoform 2);
CEP290 (protein name: centrosomal protein 290; gene: CEP290; alias 3H11Ag, BBS14, CT87, JBTS5, LCA10, MKS4, NPHP6, POC3, SLSN6, rd16), wherein a sequence encoding CEP290 is such as of SEQ ID NO: 39;
CLRN1 (protein name: clarin1; gene: CLRN1; alias RP61, USH3, USH3A), wherein a sequence encoding CLRN1 is such as of SEQ ID NO: 40;
IFT140 (protein name: intraflagellar transport 140; gene: IFT140; alias MZSDS, RP80, SRTD9, WDTC2, c305C8.4, c380F5.1, gs114), wherein a sequence encoding IFT140 is such as of SEQ ID NO: 41;
IFT172 (protein name: intraflagellar transport 172; gene: IFT172; alias BBS20, NPHP17, RP71, SLB, SRTD10, osm-1, wim), wherein a sequence encoding IFT172 is such as SEQ ID NO: 42;
IQCB1 (protein name: IQ motif containing B1; gene: IQCB1; alias NPHP5, PIQ, SLSN5), wherein a sequence encoding IQCB1 is such as of SEQ ID NO: 43;
KIZ (protein name: kizuna centrosomal protein; gene: KIZ; alias C20orf19, HT013, Kizuna, NCRNA00153, PLK1S1, RP69), wherein a sequence encoding KIZ is such as of SEQ ID NO: 44;
LCA5 (protein name: lebercilin LCA5; gene: LCA5; alias C6orf152), wherein a sequence encoding LCA5 is such as of SEQ ID NO: 45;
MAK (protein name: male germ cell associated kinase; gene: MAK; alias RP62), wherein a sequence encoding MAK is such as of SEQ ID NO: 46;
NEK2 (protein name: NIMA related kinase 2; gene: NEK2; alias HsPK21, NEK2A, NLK1, PPP1R111, RP67), wherein a sequence encoding NEK2 is such as of SEQ ID NO: 47;
OFD1 (protein name: OFD1 centriole and centriolar satellite protein; gene: OFD1; alias 71-7A, CXorf5, JBTS10, RP23, SGBS2), wherein a sequence encoding OFD1 is such as of SEQ ID NO: 48;
OFD1 (protein name: RAB28, member RAS oncogene family; gene: RAB28; alias CORD18), wherein a sequence encoding OFD1 is such as of SEQ ID NO: 49;
RP1 (protein name: RP1 axonemal microtubule associated; gene: RP1; alias ORP1, DCDC4A), wherein a sequence encoding RP1 is such as of SEQ ID NO: 50;
RP1L1 (protein name: RP1 like 1; gene: RP1L1; alias OCMD, RP88, DCDC4B), wherein a sequence encoding RP1L1 is such as of SEQ ID NO: 51;
RP2 (protein name: RP2 activator of ARL3 GTPase; gene: RP2; alias XRP2, NME10, TBCCD2, NM23-H10, DELXp11.3), wherein a sequence encoding RP2 is such as of SEQ ID NO: 52;
RPGR (protein name: retinitis pigmentosa GTPase regulator; gene: RPGR; alias COD1, CORDX1, CRD, PCDX, RP15, RP3, XLRP3, orf15), wherein a sequence encoding RPGR is such as of SEQ ID NO: 53 (RPGR isoform C) or SEQ ID NO: 54 (RPGR isoform J);
RPGRIP1 (protein name: RPGR interacting protein 1, gene: RPGRIP1; alias CORD13, LCA6, RGI1, RGRIP, RPGRIP, RPGRIP1d), wherein a sequence encoding RPGRIP1 is such as of SEQ ID NO: 55 (RPGRIP1 isoform 1) or SEQ ID NO: 56 (RPGRIP1 isoform 3);
SPATA7 (protein name: spermatogenesis associated 7; gene: SPATA7; alias HEL-S-296, HSD-3.1, HSD3, LCA3), wherein a sequence encoding SPATA7 is such as of SEQ ID NO: 57;
TOPORS (protein name: TOP1 binding arginine/serine rich protein, E3 ubiquitin ligase; gene: TOPORS; alias LUN, RP31, P53BP3, TP53BPL), wherein a sequence encoding TOPORS is such as of SEQ ID NO: 58;
TTC8 (protein name: tetratricopeptide repeat domain 8; gene: TTC8; alias BBS8, RP51), wherein a sequence encoding TTC8 is such as of SEQ ID NO: 59;
USH2A (protein name: usherin; gene: USH2A; alias RP39, US2, USH2, dJ1111A8.1), wherein a sequence encoding USH2A is such as of SEQ ID NO: 60;
WDR19 (protein name: WD repeat domain 19; gene: WDR19; alias ATD5, CED4, DYF-2, FAP66, IFT144, NPHP13, ORF26, Oseg6, PWDMP, SRTD5), wherein a sequence encoding WDR19 is such as of SEQ ID NO: 61, or ciliary protein fragment, or ciliary protein variant.
CFAP410 (protein name: cilia and flagella associated protein 410; gene: CFAP410; alias RDMS; SMDAX; LRRC76; YF5/A2; C21orf2), wherein a sequence encoding CFAP410 is such as of SEQ ID NO: 62.

In one embodiment the sequences encoding ciliary proteins are selected from SEQ ID NO: 29 to SEQ ID NO: 62, or fragments or variants thereof.

In one embodiment the SEQ ID NO: 29 to SEQ ID NO: 62 are cDNA sequences.

It is understood that the promoter according to the invention drives adequate level of expression, allowing the ciliary protein to be expressed in the correct location without spreading of the protein into the cell body.

### Vectors and Plasmids

In one embodiment, is provided a vector suitable for delivery of a nucleic acid sequence encoding a ciliary protein, or a fragment thereof, or a variant thereof to a photoceptor.

In one embodiment a vector is a viral vector.

In one embodiment, a viral vector is a recombinant adeno-associated virus (rAAV).

In one embodiment, a vector comprises a promotor that is FCBR1 promotor (F, of the FAM161A gene, CBR, Crx binding region) such as encoded by SEQ ID NO: 7, or SEQ ID NO: 63, or a fragment thereof, or a variant thereof. In another embodiment, a vector comprises a promotor that is comprising FCBR1 promotor of SEQ ID NO: 7, or SEQ ID NO: 63, or a fragment thereof, or a variant thereof.

In one embodiment, a vector comprises a promotor that is F0.4 promotor (FAM161A core promoter of 460 nucleotides) such as encoded by SEQ ID NO: 9, or SEQ ID NO: 65, or a fragment thereof, or a variant thereof. In another embodiment, a vector comprises a promotor that is comprising Core promotor of SEQ ID NO: 9, or SEQ ID NO: 65, or a fragment thereof, or a variant thereof.

In one embodiment, a vector comprises a promotor that is FCBR1-F0.4 promotor (F, of the FAM161A gene; CBR, Crx binding region 1; F0.4, FAM161A core promoter) such as encoded by SEQ ID NO: 10, or SEQ ID NO: 66, or a fragment thereof, or a variant thereof. In another embodiment, a vector has a promotor comprising a FCBR1-F0.4 promotor of SEQ ID NO: 10, or SEQ ID NO: 66, or a fragment thereof, or a variant thereof.

In one embodiment SEQ ID NO: 10 comprises SEQ ID NO: 7 and SEQ ID NO: 9. In one embodiment SEQ ID NO: 66 comprises SEQ ID NO: 63 and SEQ ID NO: 65.

In one embodiment, a vector comprises a promotor that is FCBR2 promotor (F, of the FAM161A gene; CBR, Crx binding region 2) such as encoded by SEQ ID NO: 8 or SEQ ID NO: 64, or a fragment thereof, or a variant thereof. In another embodiment, a vector has a promotor that is comprising CBR2 promotor of SEQ ID NO: 8 or SEQ ID NO: 64, or a fragment thereof, or a variant thereof.

In one embodiment, a vector comprises a promotor that is FCBR2-F0.4 promotor (CBR, Crx binding region; F0.4, FAM161A core promoter) such as encoded by SEQ ID NO: 11 or SEQ ID NO: 67, or a fragment thereof, or a variant thereof. In another embodiment, a vector has a promotor that is comprising FCBR2-Core promotor of SEQ ID NO: 11 or SEQ ID NO: 67, or a fragment thereof, or a variant thereof.

In one embodiment SEQ ID NO: 11 comprises SEQ ID NO: 8 and SEQ ID NO: 9. In one embodiment SEQ ID NO: 67 comprises SEQ ID NO: 64 and SEQ ID NO: 65.

The FCBR1-F0.4 or FCBR2-F0.4 can be referred as a promotor. In one embodiment, the promotor is FCBR1-F0.4 or FCBR2-F0.4. In another embodiment, the promotor comprises FCBR1-F0.4 or FCBR2-F0.4.

In description “F0.4” can be marked as “core”, and “FCBR1” and “FCBR2” can be marked as “CBR1” and “CBR2”, such that the “FCBR1-F0.4” and “FCBR2-F0.4” can be “CBR1-core” and “CBR2-core”.

In one embodiment, a vector comprises a promotor that is GRK1 promotor (G Protein-Coupled Receptor Kinase 1) such as encoded by SEQ ID NO: 13, or a fragment thereof, or a variant thereof. In another embodiment, a vector has a promotor that is comprising GRK1 promotor of SEQ ID NO: 13, or a fragment thereof, or a variant thereof. The GRK1 promotor may be referred herein as GRK1 or hGRK1 or GRK1p.

In one embodiment, a vector comprises an enhancer that is IRBP enhancer (Interphotoreceptor retinoid-binding protein) such as encoded by SEQ ID NO: 12, or a fragment thereof, or a variant thereof. In another embodiment, a vector has a promotor that is comprising IRBP enhancer of SEQ ID NO: 12, or a fragment thereof, or a variant thereof. The IRBP enhancer may referred herein as IRBPen (or IRBP depending on the context).

In one embodiment, a vector comprises a promotor that is IRBP-GRK1 promotor, or a fragment thereof, or a variant thereof, such as encoded by SEQ ID NO: 14. In another embodiment, a vector has a promotor that is comprising IRBP-GRK1 promotor of SEQ ID NO: 14, or a fragment thereof, or a variant thereof. The IRBP-GRK1 can be referred as a promotor. In one embodiment, the promotor is IRBP-GRK1. In another embodiment, the promotor comprises IRBP-GRK1.

In one embodiment, a vector comprises a nucleic acid sequence encoding a hFAM161A gene, or a fragment, or a variant thereof. The nucleic acid sequence encoding a hFAM161A gene is refereed herein as a transgene or gene of interest. In one embodiment, the transgene is hFAM161A gene, or a fragment, or a variant thereof. In another embodiment, the transgene comprises hFAM161A gene, or a fragment, or a variant thereof.

In one aspect, a vector comprises a nucleic acid sequence encoding a long isoform of human FAM161A gene of SEQ ID NO: 1 (hFAM161A-L) or a short isoform of human FAM161A gene of SEQ ID NO: 2 (hFAM161A-S), or a fragment, or a variant thereof.

In one aspect, a vector comprises a nucleic acid sequence encoding a long isoform of human FAM161A gene of SEQ ID NO: 1 (hFAM161A-L), or a fragment, or a variant thereof.

In one aspect, a vector comprises a nucleic acid sequence encoding a short isoform of human FAM161A gene of SEQ ID NO: 2 (hFAM161A-S), or a fragment, or a variant thereof.

In one embodiment, a vector comprises a nucleic acid sequence encoding a ciliary protein gene, or a fragment, or a variant thereof.

In one aspect, a vector comprises a nucleic acid sequence encoding a ciliary protein gene, selected from POC5 gene of SEQ ID NO: 29, NPHP4 gene of SEQ ID NO: 30, ARL6 gene of SEQ ID NO: 31, BBS1 gene of SEQ ID NO: 32, BBS2 gene of SEQ ID NO: 33, BBS9 gene of SEQ ID NO: 34, PCARE gene of SEQ ID NO: 35, CFAP418 gene of SEQ ID NO: 36, CEP164 gene of SEQ ID NO: 37 (CEP164 isoform 1) or of SEQ ID NO: 38 (CEP164 isoform 2), CEP290 gene of SEQ ID NO: 39, CLRN1 gene of SEQ ID NO: 40, IFT140 gene of SEQ ID NO: 41, IFT172 gene of SEQ ID NO: 42, IQCB1 gene of SEQ ID NO: 43, KIZ gene of SEQ ID NO: 44, LCA5 gene of SEQ ID NO: 45, MAK gene of SEQ ID NO: 46, NEK2 gene of SEQ ID NO: 47, OFD1 gene of SEQ ID NO: 48, OFD1 gene of SEQ ID NO: 49, RP1 gene of SEQ ID NO: 50, RP1L1 gene of SEQ ID NO: 51, RP2 gene of SEQ ID NO: 52, RPGR gene of SEQ ID NO: 53 (RPGR isoform C) or SEQ ID NO: 54 (RPGR isoform J), RPGRIP1 gene of SEQ ID NO: 55 (RPGRIP1 isoform 1) or SEQ ID NO: 56 (RPGRIP1 isoform 3), SPATA7 gene of SEQ ID NO: 57, TOPORS gene of SEQ ID NO: 58, TTC8 gene of SEQ ID NO: 59, USH2A gene of SEQ ID NO: 60, WDR19 gene of SEQ ID NO: 61, CFAP410 gene of SEQ ID NO: 62, or a fragment or a variant thereof.

In one aspect, a vector comprises a FCBR1-F0.4 promotor and a nucleic acid sequence encoding a hFAM161A gene, such as hFAM161A-L gene or hFAM161A-S gene, or a fragment or a variant thereof.

In one aspect, a vector comprises a FCBR2-F0.4 promotor and a nucleic acid sequence encoding a hFAM161A gene, such as hFAM161A-L gene or hFAM161A-S gene, or a fragment or a variant thereof.

In one aspect, a vector comprises an IRBP-GRK1 promotor and a nucleic acid sequence encoding a hFAM161A gene, such as hFAM161A-L gene or hFAM161A-S gene, or a fragment or a variant thereof.

In one aspect, a vector comprises a FCBR1-F0.4 promotor and a nucleic acid sequence encoding a ciliary protein gene, such as genes of POC5, NPHP4, ARL6, BBS1, BBS2, BBS9, PCARE, CFAP418, CEP164 isoform 1 or isoform 2, CEP290, CLRN1, IFT140, IFT172, IQCB1, KIZ, LCA5, MAK, NEK2, OFD1, OFD1, RP1, RP1L1, RP2, RPGR isoform C or isoform J, RPGRIP1 isoform 1 or isoform 3, SPATA7, TOPORS, TTC8, USH2A, WDR19, CFAP410, or a fragment or a variant thereof.

In one aspect, a vector comprises a FCBR2-F0.4 promotor and a nucleic acid sequence encoding a ciliary protein gene, such as genes of POC5, NPHP4, ARL6, BBS1, BBS2, BBS9, PCARE, CFAP418, CEP164 isoform 1 or isoform 2, CEP290, CLRN1, IFT140, IFT172, IQCB1, KIZ, LCA5, MAK, NEK2, OFD1, OFD1, RP1, RP1L1, RP2, RPGR isoform C or isoform J, RPGRIP1 isoform 1 or isoform 3, SPATA7, TOPORS, TTC8, USH2A, WDR19, CFAP410, or a fragment or a variant thereof.

In one aspect, a vector comprises a IRBP-GRK1 promotor and a nucleic acid sequence encoding a ciliary protein gene, such as genes of POC5, NPHP4, ARL6, BBS1, BBS2, BBS9, PCARE, CFAP418, CEP164 isoform 1 or isoform 2, CEP290, CLRN1, IFT140, IFT172, IQCB1, KIZ, LCA5, MAK, NEK2, OFD1, OFD1, RP1, RP1L1, RP2, RPGR isoform C or isoform J, RPGRIP1 isoform 1 or isoform 3, SPATA7, TOPORS, TTC8, USH2A, WDR19, CFAP410, or a fragment or a variant thereof.

In one embodiment, a vector comprises a post-transcriptional regulatory element, such as WPRE (woodchuck hepatitis virus post-transcriptional regulatory element). In one embodiment, a vector comprises a post-transcriptional regulatory element, such as SINEUP non-coding RNA (modular antisense long non-coding RNAs).

In another embodiment, a vector comprises a post-transcriptional regulatory element, such as WPRE of SEQ ID NO: 15, or a fragment thereof, or a variant thereof. In another embodiment, a vector has a post-transcriptional regulatory element that is comprising WPRE of SEQ ID NO: 15, or a fragment thereof, or a variant thereof.

In one aspect, a vector comprises a FCBR1-F0.4 promotor, a nucleic acid sequence encoding a hFAM161A gene, such as hFAM161A-L gene or hFAM161A-S gene, or a fragment or a variant thereof, and WPRE.

In one aspect, a vector comprises a FCBR2-F0.4 promotor, a nucleic acid sequence encoding a hFAM161A gene, such as hFAM161A-L gene or hFAM161A-S gene, or a fragment or a variant thereof, and WPRE.

In one aspect, a vector comprises an IRBP-GRK1 promotor, a nucleic acid sequence encoding a hFAM161A gene, such as hFAM161A-L gene or hFAM161A-S gene, or a fragment or a variant thereof, and WPRE.

In one aspect, a vector comprises a FCBR1-F0.4 promotor, a nucleic acid sequence encoding a ciliary protein gene, such as selected from gene of POC5, NPHP4, ARL6, BBS1, BBS2, BBS9, PCARE, CFAP418, CEP164 isoform 1 or isoform 2, CEP290, CLRN1, IFT140, IFT172, IQCB1, KIZ, LCA5, MAK, NEK2, OFD1, OFD1, RP1, RP1L1, RP2, RPGR isoform C or isoform J, RPGRIP1 isoform 1 or isoform 3, SPATA7, TOPORS, TTC8, USH2A and WDR19, CFAP410, or a fragment or a variant thereof, and WPRE.

In one aspect, a vector comprises a FCBR2-F0.4 promotor, a nucleic acid sequence encoding a ciliary protein gene, such as selected from gene of POC5, NPHP4, ARL6, BBS1, BBS2, BBS9, PCARE, CFAP418, CEP164 isoform 1 or isoform 2, CEP290, CLRN1, IFT140, IFT172, IQCB1, KIZ, LCA5, MAK, NEK2, OFD1, OFD1, RP1, RP1L1, RP2, RPGR isoform C or isoform J, RPGRIP1 isoform 1 or isoform 3, SPATA7, TOPORS, TTC8, USH2A and WDR19, CFAP410, or a fragment or a variant thereof, and WPRE.

In one aspect, a vector comprises an IRBP-GRK1 promotor, a nucleic acid sequence encoding a ciliary protein gene, such as selected from gene of POC5, NPHP4, ARL6, BBS1, BBS2, BBS9, PCARE, CFAP418, CEP164 isoform 1 or isoform 2, CEP290, CLRN1, IFT140, IFT172, IQCB1, KIZ, LCA5, MAK, NEK2, OFD1, OFD1, RP1, RP1L1, RP2, RPGR isoform C or isoform J, RPGRIP1 isoform 1 or isoform 3, SPATA7, TOPORS, TTC8, USH2A and WDR19, CFAP410, or a fragment or a variant thereof, and WPRE.

In one embodiment, a vector comprises a sequence selected from SEQ ID NO: 25 (AAV2/8-FCBR1-F0.4-hFAM161A-L-WPRE), or SEQ ID NO: 26 (AAV2/8-FCBR1-F0.4-hFAM161A-S-WPRE), or a fragment or a variant thereof.

In one embodiment, a vector comprises a sequence selected from SEQ ID NO: 21 (AAV2/8-FCBR2-F0.4-hFAM161A-L-WPRE) or SEQ ID NO: 17 SEQ ID NO: 22 (AAV2/8-FCBR2-F0.4-hFAM161A-S-WPRE), or a fragment or a variant thereof.

In one embodiment, a vector comprises a sequence selected from SEQ ID NO: 16 (AAV2/8-IRBP-GRK1-hFAM161A-L-WPRE) or SEQ ID NO: 17 (AAV2/8-IRBP-GRK1-hFAM161A-S-WPRE), or a fragment or a variant thereof.

It is understood that SEQ ID NO: 16 to SEQ ID NO: 28 are AAV transgene cassettes (from 5′ ITR to 3′ITR, comprising the ITR).

In one embodiment, rAAV has a capsid protein selected from the group consisting of: AAV1, AAV2, AAV3, AAV4, AAV5, AAV6, AAV7, AAV8, AAV9, AAV2/1, AAV2/2, AAV2/4, AAV2/5, AAV2/6, AAV2/7, AAV2/8 and AAV2/9. In a preferred embodiment, rAAV has a AAV2/8 or an AAV2/5 capsid protein.

In one embodiment, there is provided a mix of at least two vectors according to the invention.

In another embodiment, a mix of at least two vectors comprises a vector comprising SEQ ID NO: 25 (AAV2/8-FCBR1-F0.4-hFAM161A-L-WPRE) and SEQ ID NO: 26 (AAV2/8-FCBR1-F0.4-hFAM161A-S-WPRE), wherein preferably the two vectors are mix in 1:1 proportion. In one embodiment, there is provided a plasmid suitable for generation of a vector for delivery of a nucleic acid sequence encoding a ciliary protein, or a fragment thereof, or a variant thereof to a photoreceptor, as described herein. The plasmids according to the invention comprise the elements as described for the vectors of the invention.

In another embodiment, there is provided a method of vector production, such as using engineered cell lines, a baculovirus (BV) expression system or any transient transfection method on HEK293T cells or any other suitable cell line for AAV production, such as Sf9, or ExpresSf+ cells. It is understood that any known method of vector production can be used.

In another embodiment, there is provided a method of vector purification, such as using iodixanol gradient, ultrafiltration or chromatography separation or any other suitable method for AAV purification. It is understood that any known method of vector purification can be used.

In another embodiment, there is provided a nucleic acid sequence encoding rAAV according to the invention.

In another embodiment, there is provided a cell comprising a nucleic acid sequence encoding rAAV according to the invention.

In another embodiment, there is provided a method of vector production that involves transfecting HEK293 cells with either two or three plasmids, wherein one is encoding the gene of interest as decried herein, one is carrying the AAV rep/cap genes, and another is containing helper genes provided by either adeno or herpes viruses.

### Compositions, Administration and Kits

In one embodiment, there is provided a composition comprising at least one vector of the invention comprising a nucleic acid sequence encoding a FCBR1-F0.4 promotor or a FCBR2-F0.4 promotor or an IRBP-GRK1 promoter, and a ciliary protein, such as hFAM161A, or a fragment or a variant thereof, and at least one physiologically acceptable vehicle or carrier.

Exemplary physiologically acceptable carriers, particularly one suitable for administration to the eye, include sterile pyrogen-free phosphate buffered saline, sterile pyrogen-free TSSM-buffer (tromethamine 20 mM, NaCl 100 mM, sucrose 10 mg/mL, and mannitol 10 mg/mL) and the like.

In another embodiment, there is provided a composition comprising at least one vector of the invention carrying a nucleic acid sequence encoding a FCBR1-F0.4 promotor or a FCBR2-F0.4 promotor or an IRBP-GRK1 promoter, and a ciliary protein, such as hFAM161A, or a fragment or a variant thereof, and at least one pharmaceutically acceptable vehicle or carrier. Exemplary pharmaceutically acceptable vehicle or carrier, particularly one suitable for administration to the eye, include buffered saline or other buffers, e.g., HEPES, to maintain pH at appropriate physiological levels, and, optionally, other medicinal agents, pharmaceutical agents, stabilizing agents, buffers, carriers, adjuvants, diluents, and the like.

Compositions and pharmaceutical compositions of the invention may contain one or more vectors of the invention comprising any elements as described herein. Compositions and pharmaceutical compositions according to the invention are particularly suitable for administration to the eye, e.g., by subretinal injection or intravitreous injection, but also by systemic administration.

In one embodiment, if the vector is to be stored long-term, it may be frozen in the presence of a cryoprotectant, e.g. glycerol or Tween20.

In one embodiment, there is provided a composition or a pharmaceutical composition according to the invention, suitable for administration to the eye, preferably by subretinal injection.

The compositions of the invention may be delivered in a volume of from about 50 μL to about 1 ml, including all numbers within the range, depending on the size of the area to be treated, the viral titer used, the route of administration, and the desired effect of the method. In one embodiment, the volume is from about μL 100 to about 300 μL.

An effective concentration of a rAAV according to the invention ranges between about 10⁸ and 10¹³ vector genomes per milliliter (vg/mL). The rAAV infectious units are measured as according to known methods. Preferably, the concentration is from about 1.5×10⁹ vg/mL to about 5×10¹⁴ vg/mL. In another embodiment, the effective concentration is about 1.5×10¹² vg/mL to about 1.5×10¹⁴ vg/mL. It is desirable that the lowest effective concentration of rAAV be utilized in order to reduce the risk of undesirable effects, such as toxicity, retinal dysplasia, and detachment. Still other dosages in these ranges may be selected considering the physical state of the subject being treated, the age of the subject, the particular ocular disorder and the degree to which the disorder, if progressive, has developed.

According to one aspect, the invention relates to a kit for carrying out a method or a use according to the invention.

According to one embodiment, there is provided a kit comprising at least one rAAV according to the invention or a combination thereof and an instruction of use thereof.

### Methods and Uses

Retinal ciliopathy, a FAM161A-associated retinopathy, such as a retinitis pigmentosa-28 (RP28), is associated with many retinal changes. These include a loss of photoreceptor structure and/or function, thinning or thickening of the outer nuclear layer (ONL), thinning or thickening of the outer plexiform layer (OPL), disorganization followed by loss of rod and cone outer segments, shortening of the rod and cone inner segments, retraction of bipolar cell dendrites, thinning or thickening of the inner retinal layers including inner nuclear layer, inner plexiform layer, ganglion cell layer and nerve fiber layer, opsin mislocalization, overexpression of neurofilaments, loss of ERG function, loss of visual acuity and contrast sensitivity, and loss of visually guided behavior. In one embodiment, the invention provides a method of modifying, preventing, arresting progression of, or ameliorating any of the retinal changes associated with retinal ciliopathy. As a result, the subject's vision is improved, or vision loss is delayed or arrested and/or ameliorated.

In one embodiment, there is provided a method of modifying, preventing, delaying, or arresting progression of or ameliorating vision loss associated with a retinal ciliopathy, such as a FAM161A-associated retinopathy, preferably such as RP28, in a subject in need thereof, comprising administrating an effective amount of a rAAV according to the invention or a composition thereof.

In one embodiment, there is provided a method of treatment of a retinal ciliopathy, such as a FAM161A-associated retinopathy, preferably such as RP28, in a subject in need thereof, comprising administrating an effective amount of a rAAV according to the invention or a composition thereof.

In another embodiment, there is provided a method of improving photoreceptor structure, in a subject in need thereof, comprising administrating an effective amount of a rAAV according to the invention or a composition comprising a rAAV according to the invention.

In a particular embodiment, there is provided a method of improving photoreceptor outer segment structure, in a subject in need thereof, comprising administrating an effective amount of a rAAV according to the invention or a composition thereof.

In another embodiment, there is provided a method of inducing photoreceptor cilium protein stabilization by expressing at least one ciliary protein in the photoreceptor's cilium, comprising administrating an effective amount of a rAAV according to the invention or a composition thereof.

The methods according to the invention include administering to the subject in need thereof an effective amount of a rAAV according to the invention or of a composition comprising thereof, preferably by subretinal injection.

According to one embodiment, there is provided a rAAV according to the invention for use as a medicament.

According to one embodiment, there is provided a rAAV according to the invention or a composition comprising thereof for use in the modification, prevention, delaying, arresting progression of, or ameliorating vision loss associated with a retinal ciliopathy, such as a FAM161A-associated retinopathy, preferably such as RP28.

According to one embodiment, there is provided a rAAV according to the invention or a composition comprising thereof for use in the treatment of a retinal ciliopathy, such as a FAM161A-associated retinopathy, preferably such as RP28.

According to one embodiment, the invention provides a rAAV according to the invention or a composition comprising thereof for use in the treatment of RP28.

In another embodiment, there is provided a rAAV according to the invention or a composition comprising thereof for use in the improvement of photoreceptor structure.

In a particular embodiment, there is provided a rAAV according to the invention or a composition thereof for use in the improvement of photoreceptor outer segment structure.

In another embodiment, there is provided a rAAV according to the invention or a composition thereof for use in the induction of photoreceptor cilium protein stabilization.

The uses according to the invention include administering to the subject in need thereof an effective amount of a rAAV according to the invention or of a composition comprising a rAAV according to the invention, preferably by subretinal injection, or by intravitreal injection, or by intravenous injection.

According to another embodiment, the invention provides a use of a rAAV according to the invention for the preparation of a composition for prevention, modification, arresting progression of, or ameliorating vision loss associated with a retinal ciliopathy, such as a FAM161A-associated retinopathy, preferably such as RP28 and/or the treatment of a retinal ciliopathy, such as RP28 and/or the improvement of photoreceptor structure, and/or the improvement of photoreceptor outer segment structure and/or for the induction of photoreceptor cilium protein stabilization.

In one embodiment, an effective amount of a rAAV according to the invention or a composition according to the invention is administered only to one or more regions of the eye, e.g., those which have retained photoreceptors. In another embodiment, the composition is administered to the entire eye.

It is understood that transduction with rAAV leads to expression of the product of the gene encoded in rAAV, in particular at least one ciliary protein. It is understood that expression of at least one ciliary protein results in photoreceptor cilium stabilization and improved photoreceptor function. As a result, the subject's vision is improved, or vision loss is arrested and/or ameliorated.

In yet another embodiment of the invention, any of the above-described methods is performed in combination with another, or secondary, therapy. The therapy may be any now known, or as yet unknown, therapy which helps prevent, arrest or ameliorate a retinal ciliopathy, such as RP28, or any of the described effects associated therewith. The secondary therapy can be administered before, concurrent with, or after administration of the rAAV described above.

In certain embodiments of the methods of this invention, an effective amount of a rAAV according to the invention or of a composition according to the invention is administered to the subject by subretinal injection.

In one aspect, photoreceptor function may be assessed using the functional studies known for the skilled in the art and as for example described in the examples below, e.g., ERG, FST, or microperimetry or pupil light response or fMRi, which are conventional in the art. As used herein “photoreceptor function loss” means a decrease in photoreceptor function as compared to a normal, non-diseased eye or the same eye at an earlier time point. As used herein, “increase photoreceptor function” means to improve the function of the photoreceptors or increase the number or percentage of functional photoreceptors as compared to a diseased eye (having the same ocular disease at the same stage), the same eye at an earlier time point, a non-treated portion of the same eye, or the contralateral eye of the same subject. As used herein, “maintain photoreceptor function” means to main the function of the photoreceptors and to prevent the photoreceptors' degeneration.

In another aspect, the invention provides a method of improving photoreceptor structure in a subject. As used herein “improving photoreceptor structure” (in the region of the retina that is treated) refers to decrease in shortening and loss of outer segments (OS), and/or a maintenance in outer nuclear layer (ONL) thickness, and/or arresting or decreasing progression of ONL thickening (due to edema) or thinning (due to photoreceptor loss), across the entire retina, in the central retina, or the periphery (corresponding to the treated area); increase or decrease in outer plexiform layer (OPL) thickness, or arresting progression of OPL thickening or thinning, across the entire retina, in the central retina, or the periphery (corresponding to the treated area); decrease in rod and cone inner segment (IS) shortening; decrease in bipolar cell dendrite retraction, or an increase in bipolar cell dendrite length or amount; and reversal of opsin mislocalization.

For each of the described methods or uses, the treatment may be used to prevent the occurrence of retinal damage or to rescue eyes having mild or advanced disease. As used herein, the term “rescue” means to prevent or delay progression of the disease to total blindness, prevent spread of damage to uninjured photoreceptor cells or to reduce damage in injured photoreceptor cells. Thus, in one embodiment, an effective amount of a rAAV according to the invention or a composition comprising a rAAV according to the invention is administered before disease onset. In another embodiment, an effective amount of a rAAV according to the invention or a composition comprising a rAAV according to the invention is administered after the initiation of a disease. In another embodiment, an effective amount of a rAAV according to the invention or a composition comprising a rAAV according to the invention is administered prior to the initiation of photoreceptor loss. In another embodiment, an effective amount of a rAAV according to the invention or a composition comprising a rAAV according to the invention is administered after initiation of photoreceptor loss.

In yet another embodiment, an effective amount of a rAAV according to the invention or a composition comprising a rAAV according to the invention is administered when a subject has 10% or more, or 20% or more, or 30% or more, or 40% or more, or 50% or more, or 60% or more, or 70% or more, or 80%, or 90% or more photoreceptors that are functioning or remaining, as compared to a non-diseased eye. In one embodiment, an effective amount of a rAAV according to the invention or a composition comprising a rAAV according to the invention is administered only to one or more regions of the eye, e.g., those which have retained photoreceptors. In another embodiment, the composition is administered to the entire eye.

In another embodiment, there is provided a method of treating or preventing retinal ciliopathy in a subject in need thereof. The method comprises administering to the subject an effective amount of a rAAV according to the invention or a composition comprising a rAAV according to the invention. For example, the method comprises:
identifying a subject having, or at risk of developing a retinal ciliopathy, such as RP28;
performing genotypic analysis and identifying at least one mutation in the retinal ciliopathy-related genes;
performing non-invasive retinal imaging and/or functional studies and identifying areas of retained photoreceptors to be targeted for therapy; and
administering to the subject an effective amount of a rAAV according to the invention or a composition comprising a rAAV according to the invention; whereby retinal ciliopathy is modified, prevented, arrested or ameliorated. Genotypic analysis is routine in the art.

### Patients

In one embodiment, a subject according to the invention includes a mammalian subject, in particular human, suffering from or susceptible to suffer from a retinal ciliopathy, such as a FAM161A-associated retinopathy, such as a retinitis pigmentosa-28 (RP28).

In one embodiment, a subject according to the invention is suffering from RP28.

In one embodiment, a subject has shown clinical signs of RP28 such as visual impairment and associated retina degeneration.

In another embodiment, a subject according to the invention is susceptible to suffer from RP28.

In one embodiment, a subject has not shown clinical signs of RP28.

In a particular embodiment, a subject has a genetic mutation associated with RP28.

In another particular embodiment, a subject having, or at risk of developing RP28 has pathogenic variants in the gene FAM161A. Subjects at risk of developing RP28 include those with a family history of RP28, those with one or more confirmed mutations in both alleles of the FAM161A gene.

In another embodiment, a subject according to the invention is undergoing treatment for RP28.

In one embodiment, a retinal ciliopathy is a disease selected from a retinitis pigmentosa (RP), retinitis pigmentosa such retinitis pigmentosa-28 (RP28), FAM161A-associated RP, BBS (Bardet-Biedl syndrome), SLS (Senior-Løken syndrome), JBS (Joubert syndrome), MKS (Meckel-Gruber syndrome), USH (Usher syndrome), JATD (Jeune asphyxiating thoracic dystrophy), MZSDS (Mainzer-Saldino syndrome), OFD (oral-facial-digital syndrome), OMD (occult macular dystrophy), CRD (cone-rod dystrophy), LCA (Leber congenital amaurosis), CED (cranioectodermal dysplasia, also known as Sensenbrenner syndrome).

In one embodiment, a subject according to the invention is suffering from a retinal ciliopathy, such as a retinal ciliopathy listed herein.

In another embodiment, a subject according to the invention is susceptible to suffer from a retinal ciliopathy, such as a retinal ciliopathy listed herein. In one embodiment, a subject has not shown clinical signs of a retinal ciliopathy. Subjects at risk of developing a retinal ciliopathy include those with a family history of a retinal ciliopathy, those with one or more confirmed mutations in the retinal ciliopathy-related gene, offspring of female carriers of a retinal ciliopathy-related gene mutation in both alleles.

In another embodiment, a subject according to the invention is undergoing treatment for a retinal ciliopathy, such as a retinal ciliopathy listed herein.

In one embodiment, a subject has 10% or more, or 20% or more, or 30% or more, or 40% or more, or 50% or more, or 60% or more, or 70% or more, or 80%, or 90% or more photoreceptor damage/loss.

References cited herein are hereby incorporated by reference in their entirety. The present invention is not to be limited in scope by the specific embodiments and drawings described herein, which are intended as single illustrations of individual aspects of the invention, and functionally equivalent methods and components are within the scope of the invention. The examples illustrating the invention are not intended to limit the scope of the invention in any way.

### EXAMPLES

Abbreviation list: 661W—mouse photoreceptor cell line; Ac-tub—acetylated tubulin; ARPE19—human RPE; CBR—Crx binding region; F0.4—FAM161A core promoter (or Core); ERG—Electroretinogram; HEK293T—human embryonic kidney cells; GRK1—human G protein-coupled receptor kinase 1; HL (hFAM161A-L)—human long FAM161A protein isoform; HS (hFAM161A-S)—human short FAM161A protein isoform; hTERT-RPE1—human immortalized RPE cells; IRBP—interphotoreceptor retinoid-binding protein; ML (mFam161a-L)—mouse long Fam161a protein isoform; ML1 (mFam161a-L1)—mouse long 1 Fam161a protein isoform; MS (mFam161a-S)—mouse short Fam161a protein isoform; MS1 (mFam161a-S1)—mouse short 1 Fam161a protein isoform; ONL—outer nuclear layer; WPRE—Woodchuck Hepatitis Virus (WHP) Post-transcriptional Regulatory Element.

### Example 1: Generation of Different Plasmids

Several plasmids were constructed to generate different AAV2/8 vectors coding for either the long or the short isoform of human FAM161A gene or mouse Fam161a gene as described below.

### Material and Methods

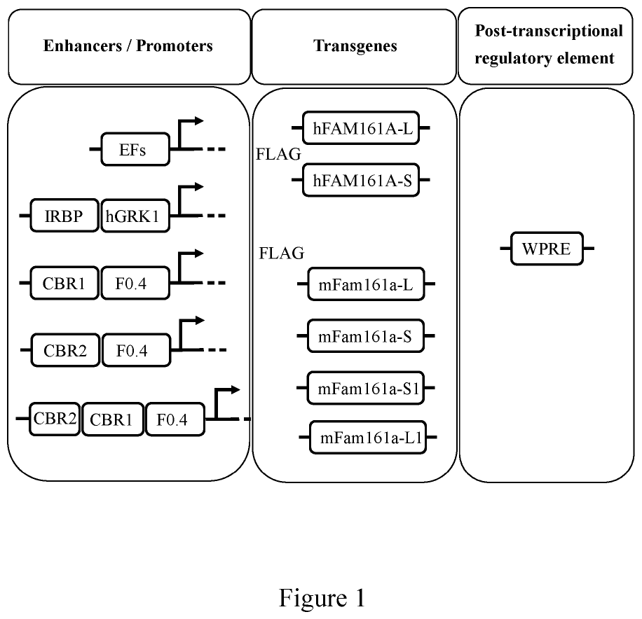

Plasmids: Several plasmids were constructed to generate different AAV2/8 vectors coding for either the long isoform of human FAM161A gene (hFAM161A-L (CCDS56120.1), SEQ ID NO: 1, refereed herein as HL) or the short isoform of human FAM161A gene (hFAM161A-S (CCDS42687.2), SEQ ID NO: 2, refereed herein as HS). Promoter sequences were PCR amplified from human or mouse DNA. The human and mouse cDNA were RT-PCR amplified from human or mouse RNA respectively. The different AAV transgene cassettes were cloned using either recombination (MultiSite Gateway® system from Invitrogen), overlap extension PCR (such as In-fusion cloning) or conventional restriction enzyme-ligation methods. As proof-of-concept validation, four mouse Fam161a isoforms and the two human isoforms were FLAG-tagged and cloned into plasmids as well. The four mice Fam161a isoforms were mFam161a-L (SEQ ID NO: 3, refereed herein as ML), mFam161a-L1 (SEQ ID NO: 4, refereed herein as ML1), mFam161a-S (SEQ ID NO: 5, refereed herein as MS), mFam161a-S1 (SEQ ID NO: 6, refereed herein as MS1) Several different cis-regulatory elements were tested including the GRK1 (human G protein-coupled receptor kinase 1) promoter in the presence of the IRBP (interphotoreceptor retinoid-binding protein) enhancer, endogenous human FAM161A promoter fragments (FCBR1-F0.4 and FCBR2-F0.4) (CBR, Crx binding region in the FAM161A gene (F), thus FCBR1 or FCBR2); F0.4, FAM161A core promoter) and the RNA stabilizer WPRE (Woodchuck Hepatitis Virus (WHP) Post-transcriptional Regulatory Element). FIG. 1 shows possible constructs combinations. Note that F0.4 can be marked as “core” and FCBR1 and FCBR2 can be marked as CBR1 and CBR2, such that the FCBR1-F0.4 and FCBR2-F0.4, can be CBR1-core and CBR2-core.

The sequences for cis-regulatory elements are as follows: FCBR1 is of SEQ ID NO: 7 or SEQ ID NO: 63, FCBR2 is of SEQ ID NO: 8 or SEQ ID NO: 64, F0.4 is of SEQ ID NO: 9 or SEQ ID NO: 65, FCBR1-F0.4 is of SEQ ID NO: 10 or SEQ ID NO: 66, FCBR2-F0.4 is of SEQ ID NO: 11 or SEQ ID NO: 67, IRBP is of SEQ ID NO: 12, GRK1 of SEQ ID NO: 13, IRBP-GRK1 is of SEQ ID NO: 14, WPRE is of SEQ ID NO: 15.

The SEQ ID NO: 7 for FCBR1 differs from the sequence FCBR1 of SEQ ID NO: 63 found in database in that 2608 a>g (wherein “>” means that one nucleotide was exchanged for another one; here a was exchanged to g).

The SEQ ID NO: 8 for FCBR2 differs from the sequence FCBR2 of SEQ ID NO: 64 found in database in that 1862 a>g and 2141 a>g.

The SEQ ID NO: 9 for F0.4 differs from the sequence F.04 of SEQ ID NO: 65 found in database in that 2881 t>c, 2954 a>g, and 3226 t>c.

The produced AAV2/8 vectors were used in below experiments and were as follows:

(1)
(SEQ ID NO: 16)
AAV2/8-IRBP-GRK1-hFAM161A-L-WPRE
(comprising hFAM161A-L (HL) of SEQ ID NO: 1);
(2)
(SEQ ID NO: 17)
AAV2/8-IRBP-GRK1-hFAM161A-S-WPRE
(comprising hFAM161A-S (HS) of SEQ ID NO: 2);
(3)
(SEQ ID NO: 18)
AAV2/8-IRBP-GRK1-mFam161a-L-WPRE
(comprising mFam 161a-L (ML) of SEQ ID NO: 3);
(4)
(SEQ ID NO: 19)
AAV2/8-IRBP-GRK1-mFam161a-S-WPRE
(comprising mFam161a-S (MS) of SEQ ID NO: 5);
(5)
(SEQ ID NO: 20)
AAV2/8-IRBP-GRK1-mFam161a-S1-WPRE
(comprising mFam16la-S (MS1) of SEQ ID NO: 6);
(6)
(SEQ ID NO: 21)
AAV2/8-FCBR2-F0.4-hFAM161A-L-WPRE
(comprising hFAM161A-L (HL) of SEQ ID NO: 1);
(7)
(SEQ ID NO: 22)
AAV2/8-FCBR2F0.4-hFAM161A-S-WPRE
(comprising hFAM161A-S (HS) of SEQ ID NO: 2);
(8)
(SEQ ID NO: 23)
AAV2/8-FCBR2-F0.4-mFam161a-L-WPRE
(comprising mFam 161a-L (ML) of SEQ ID NO: 3);
(9)
(SEQ ID NO: 24)
AAV2/8-FCBR2-F0.4-mFam161a-L1-WPRE
(comprising mFam161a-L 1 (ML1) of SEQ ID NO: 4);
(10)
(SEQ ID NO: 25)
AAV2/8-FCBR1-F0.4-hFAM161A-L-WPRE
(comprising hFAM161A-L (HL) of SEQ ID NO: 1);
(11)
(SEQ ID NO: 26)
AAV2/8-FCBR1-F0.4-hFAM161A-S-WPRE
(comprising hFAM161A-S (HS) of SEQ ID NO: 2);
(12)
(SEQ ID NO: 27)
AAV2/8-FCBR1-F0.4-mFam161a-L-WPRE
(comprising mFam 161a-L (ML) of SEQ ID NO: 3);
(13)
(SEQ ID NO: 28)
AAV2/8-FCBR1-F0.4-mFam161a-L1-WPRE
(comprising mFam161a-L 1 (ML1) of SEQ ID NO: 4).

The SEQ ID NO: 16 to SEQ ID NO: 28 were AAV-transgene cassettes from 5′ ITR to 3′ITR comprising the ITR.

### Example 2: In Vitro FAM161A Protein Expression and Function

Several plasmids were constructed to generate different plasmids coding for either the human FAM161A or mouse Fam161a isoforms under the control of the GRK1 promoter in the presence of the IRBP enhancer or the ubiquitous promoter EFs (elongation 1 factor) short promoter and the RNA stabilizer WPRE. Subsequently, FAM161A protein expression was observed in vitro in mouse photoreceptor and human cell lines.

### Material and Methods

Additional expression plasmids were generated as above with the EFs promoter to express the transgene in vitro in different cell lines. The (1)—SEQ ID NO: 16, (2)—SEQ ID NO: 17, (3)—SEQ ID NO: 18, (5)—SEQ ID NO: 20 and (6)—SEQ ID NO: 21 sequences served to replace the promoter by EFs and were used to test the protein cellular fate of the different FAM161A isoforms.

As the anti-FAM161A antibody does not recognize well the mouse proteins, the mouse FAM161A isoforms (ML, MS, MS1) were also FLAG-tagged in order to be accurately detected by the anti-FLAG antibody. Transfection of the different plasmids using lipofectamine 3000 was performed in 661W mouse photoreceptor cell line, in HEK293T (human embryonic kidney), ARPE19 (human RPE) and hTERT-RPE1 (human immortalized RPE) cells. Either cells were extracted 3 days after transfection using sonication in RIPA (radioimmunoprecipitation assay) buffer for Western blot analysis or were fixed on coverslip in PAF 4% 3 days after transfection for immunocytochemistry analysis. Antibodies raised against FAM161A (Sigma HPA032119, 1:2000), Glyceraldehyde-3-Phosphate Dehydrogenase (GAPDH, Chemicon MAB 374, 1:5000) and Flag® tag (Sigma F1804, clone M2, 1:2500), also known as the DYKDDDDK-tag ((D=Aspartic acid; K=Lysine; Y=Tyrosine) were used for Western blot. Antibodies raised again Flag® tag (Sigma F1804, clone M2, 1:2500) and acetylated tubulin (Sigma T7451, 1:1000) were used for immunohistochemistry.

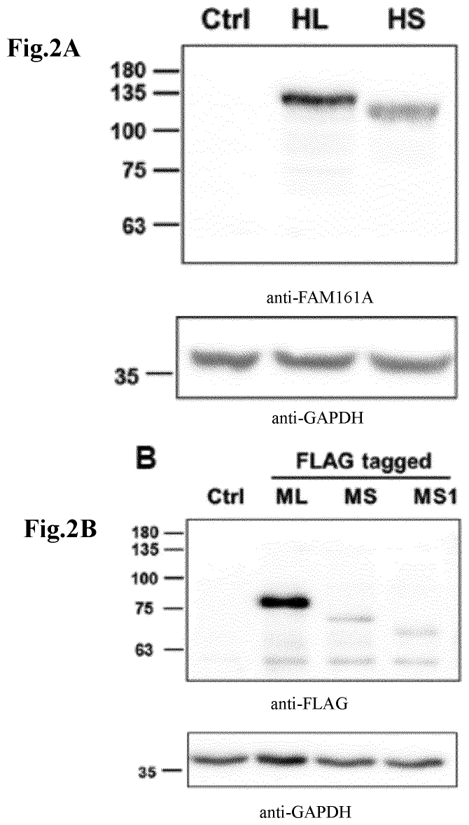

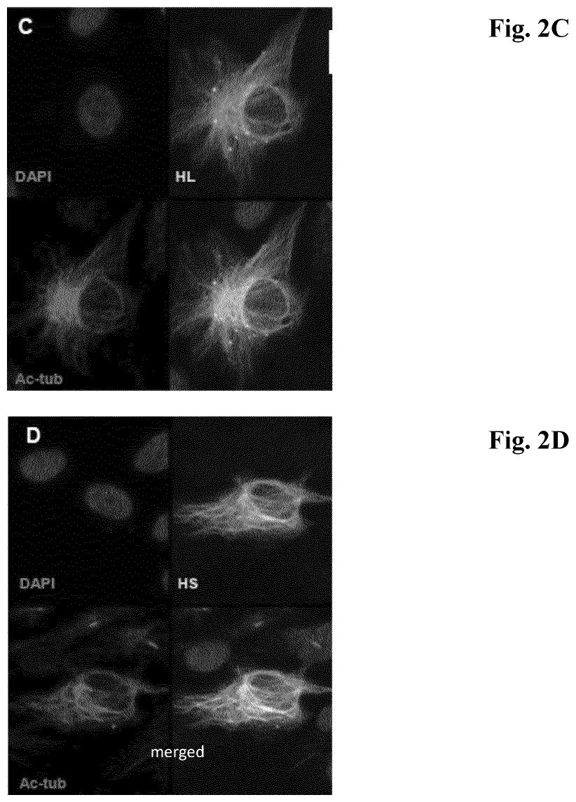

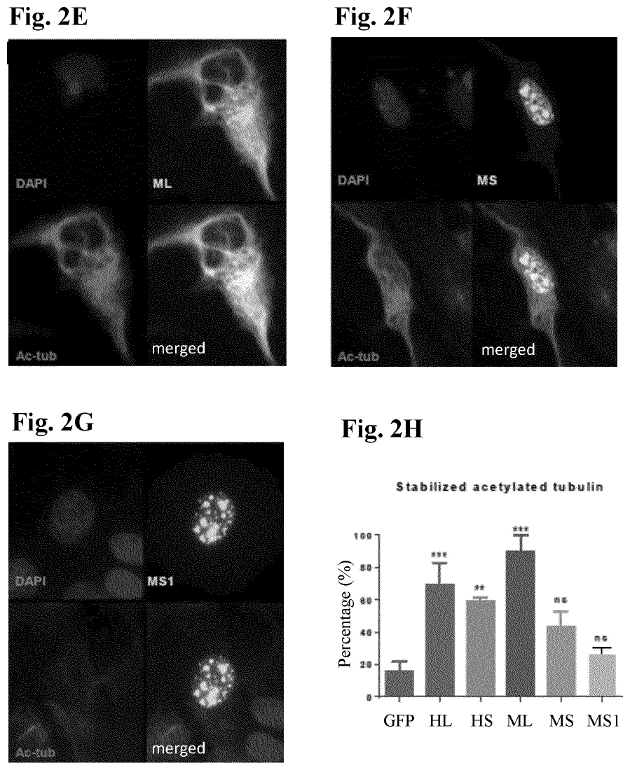

FAM161A protein expression: Plasmids were first tested in vitro in the 661W and in HEK293T to validate isoform expression by EFs or IRBP-GFK expressing plasmids. Western blot to detect FAM161A-L (HL) and hFAM161A-S(HS) after transfection of 661W with the EFs construct (FIG. 2A) or after transfection of HEK293T with the IRBP-GFK1 construct (data not shown) confirmed the different size of the two human isoforms. The same approach was used to detect mFam161a-L (ML), mFam161a-S(MS) and mFam161a-S1 (MS1) in HEK293T after transfection with the IRBP-GFK1 construct (SEQ ID NO: 18, 19, 20) (FIG. 2B). The cellular localization of the different isoforms (i.e., hFAM161A-L (HL), hFAM161A-S(HS), mFam161a-L (ML), mFam161a-S(MS), mFam161a-S1 (MS1)) were then detected by immunocytochemistry after transfection in hTERT-RPE1 cells (FIG. 2 C-G) or ARPE-19 cells. Interestingly, while the human isoforms (HL, HS) as well as mouse long isoform (ML) were expressed along the cytoskeleton (the level of Ac-tub corresponds to the level of FAM161A, see FIGS. 2E and 2H), MS and MS1 isoforms were exclusively expressed in the nucleus or surrounding the nucleus. The high expression of human FAM161A proteins and mouse L protein, as attested by their strong expression in the cell and their general distribution, resulted in an increased expression of acetylated alpha-tubulin (Ac-tub, FIG. 2C, 2D, 2E, 2H) that followed the pattern of FAM161A distribution (FIG. 2C to 2E). Quantification of stabilized acetylated tubulin as percentage of transfected cells (ARPE19) demonstrated that FAM161A participates in microtubule stabilization (FIG. 2H). Since the mouse short isoforms (MS and MS1) showed a particular sub-cellular localization in the nucleus and failed to induce cytoskeleton stabilization (FIG. 2H), an additional mouse isoform L1 has been cloned from mouse retina to be matched with the mouse L isoform (mFam161a-L1 (SEQ ID NO: 4).

These in vitro experiments confirmed that both EFs and IRBP-GRK1 can drive expression of the human and mouse isoforms and that overexpression of hFAM161A-L and -S as well as mFAM161A-L extend and stabilize microtubule network. Nevertheless, overexpression of FAM161A in the cell body seems to reorganize the whole cytoskeleton thus altering the photoreceptor function (see in vivo data below). These results suggest that low/physiological and local expression of FAM161A needs to be achieved in vivo to obtain correct FAM161A protein localization in the cilium, and to obtain adequate function.

### Example 3: Generation of Different AAV2/8 Vectors with IRBP-GRK1 and Murine Fam161a Isoforms and In Vivo Expression in Rod and Cone Photoreceptors

Material and methods. Electroretinography: Full field ERG (FFERG) was performed on anesthetized animals after overnight dark adaptation using a Ganzfeld dome and a computerized system (Espion E2, Diagnosys LLC, Littleton, MA). Briefly, pupils were dilated, and gold-wire active electrodes were placed on the central cornea. A reference electrode was placed on the tongue and a needle ground electrode was placed intramuscularly in the hip area. Dark-adapted rod and mixed cone-rod as well as light-adapted 1 Hz and 16 Hz cone flicker responses to a series of white flashes of increasing intensities (0.00008-9.6 cd·s/m2) were recorded. All ERG responses were filtered at 0.3-500 Hz, and signal averaging was applied.

Optical Coherence Tomography (OCT) and Fundus auto-fluorescence (FAF): Retinal structure was studied in-vivo by OCT b-scan 30-degree test, infrared (IR) and fundus auto-fluorescence (FAF) imaging (SPECTRALIS, Heidelberg). OCT is a noninvasive technology used to obtain cross sectional images of the retina with very high resolution. Additional two methods Infra-Red (IR) and Fundus-Auto-Fluorescence (FAF) enable to evaluate retinal degeneration status. IR imaging enables to detect retinal pathologies and especially specific features such as intraretinal fluid, chorioretinal atrophy and retinal pigment epithelium tear. FAF imaging focuses on fluorescent properties of the pigments in the retina and enable to detect retinal pathologies and especially to monitor debris accumulation in the RPE which can eventually lead to photoreceptor degeneration.

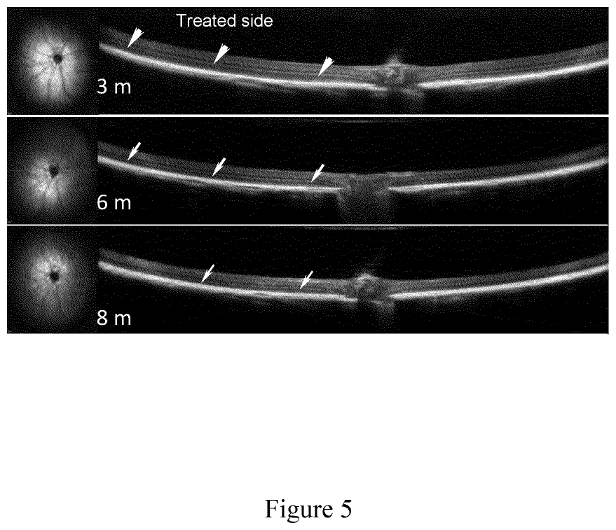

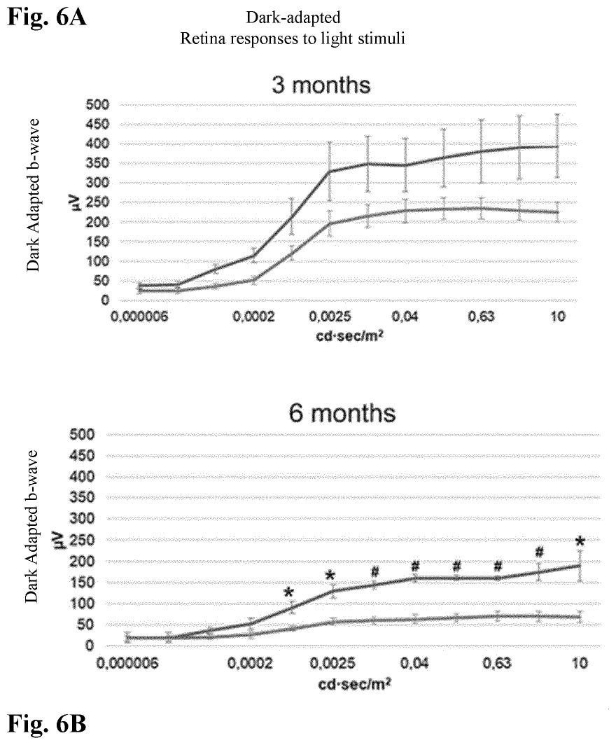

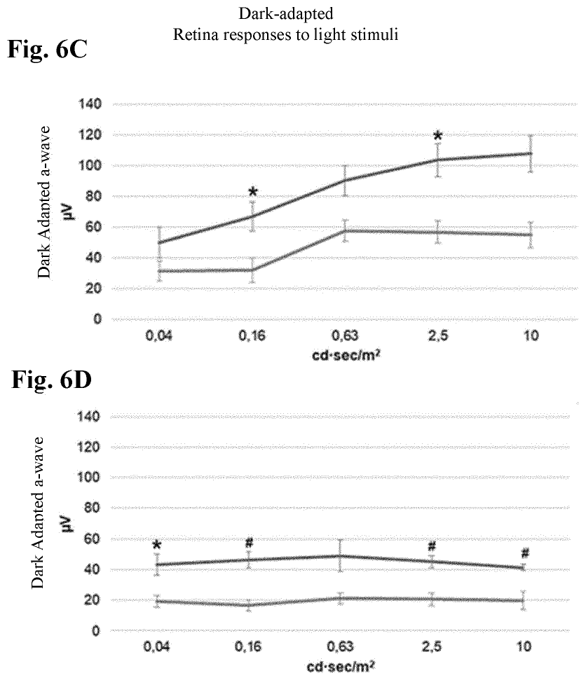

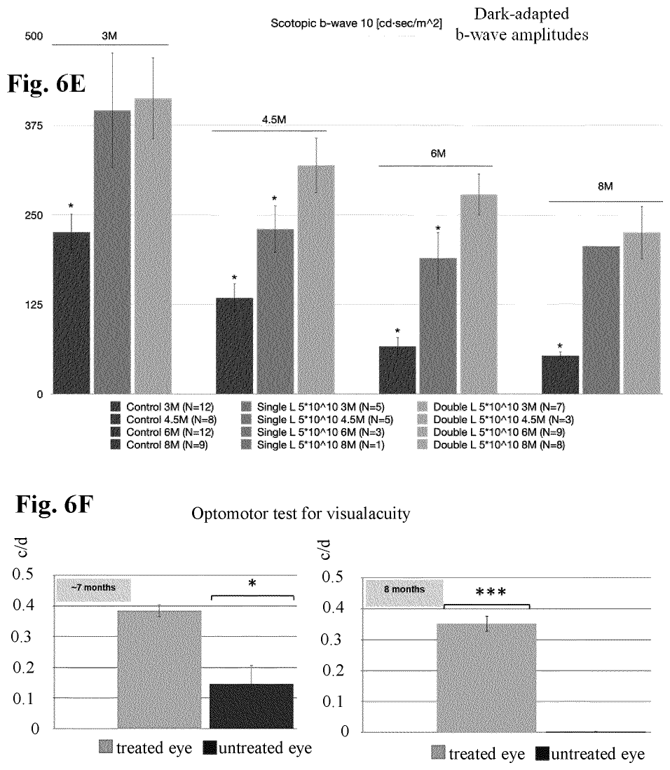

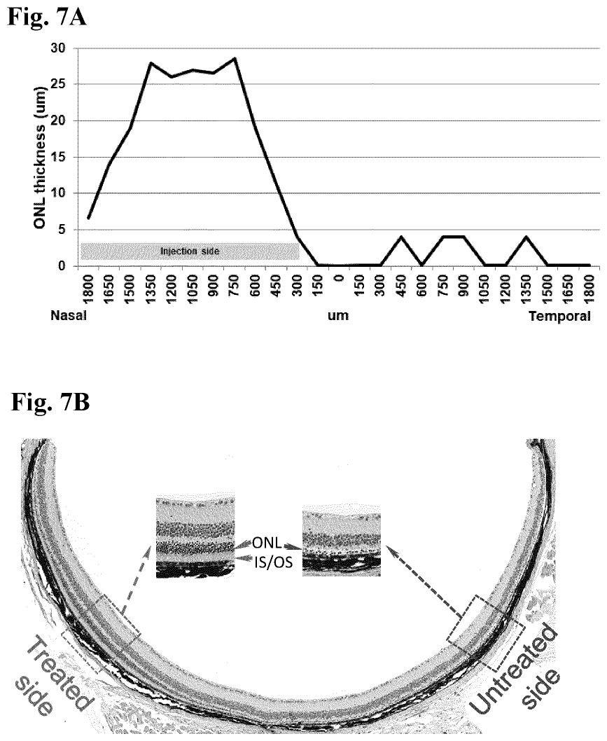

Similar experiments as in Example 4 (see Vectors) were performed using vectors coding for the murine Fam161a isoforms. In a set of experiment Fam161a^{-/-} mice were treated with the AAV2/8-IRBP-GRK1-mFam161a-L-WPRE (3) at PN31 with 0.5E11 VG and 0.001% pluronic acid in one eye (one single injection, or a double to cover the maximum of the retina area were made), the fellow eye serving as untreated control. Retina activity was assessed by electroretinogram (ERG) recording with various increasing light intensity stimulations. Examples are provided in FIGS. 6A to 6D. ERG recordings were taken at 2-, 3.5-5, and 8 months p.i. In mice, the maximum B-wave ERG responses at 3 months p.i. was well maintained as compared to non-treated mice (dark column) and this trend continue until at least 8 months p.i. The response slightly decreased because the non-treated area degenerated (FIGS. 6 and 7). Visual acuity was markedly improved 8 mpi (FIG. 6F). Optical coherence tomography (OCT) images aiming to assess retina structure in vivo were taken at 3, 6, and 8 months p.i. (4, 7 and 9 months of age respectively), and revealed a well-preserved retina ONL (black band pointed by the white arrow) at the vector treated area whereas a marked thickness decrease was observed in the non-treated area (FIG. 5).

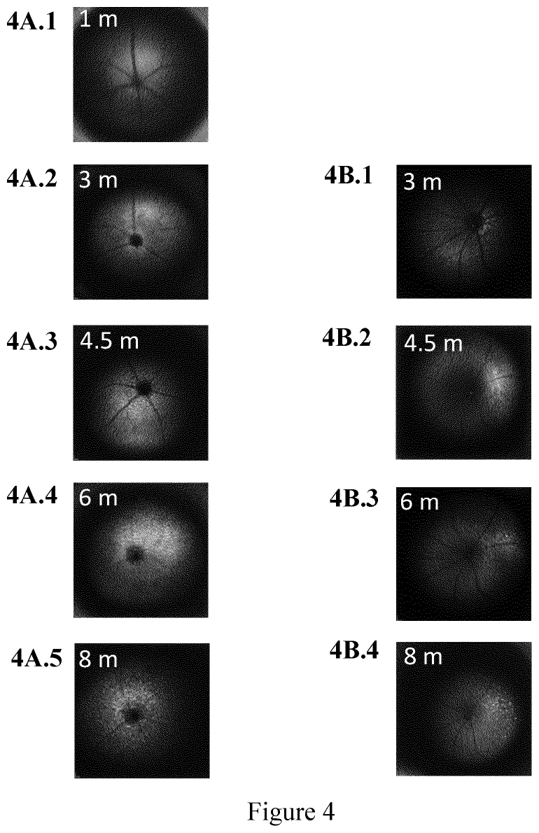

Similarly, FAF images 2—, 3.5-, 5-, and 7-month p.i. showed evidence for preserved retina in the injected eye while the non-injected eye showed the typical funduscopic finding of degenerating retina (FIG. 4).

In conclusion, gene augmentation therapy of a Fam161a^{-/-} mouse model showed improved retinal function and structure following a single injection of AAV2/8-IRBP-GRK1-mFam161a-L.

### Example 4: Generation of Different AAV2/8 Vectors with IRBP-GRK1 and Human FAM161a Isoforms and In Vivo Expression in Rod and Cone Photoreceptors

Two different AAV2/8 vectors coding for either the long or the short isoform of human FAM161A were produced as described. Subsequently, FAM161A protein expression and function in rod and cone photoreceptors was observed.

### Material and Methods

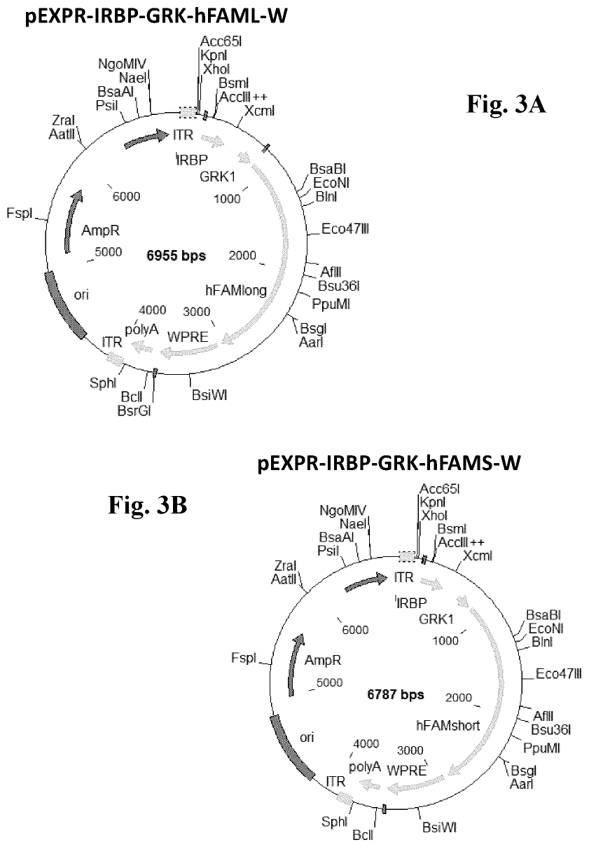

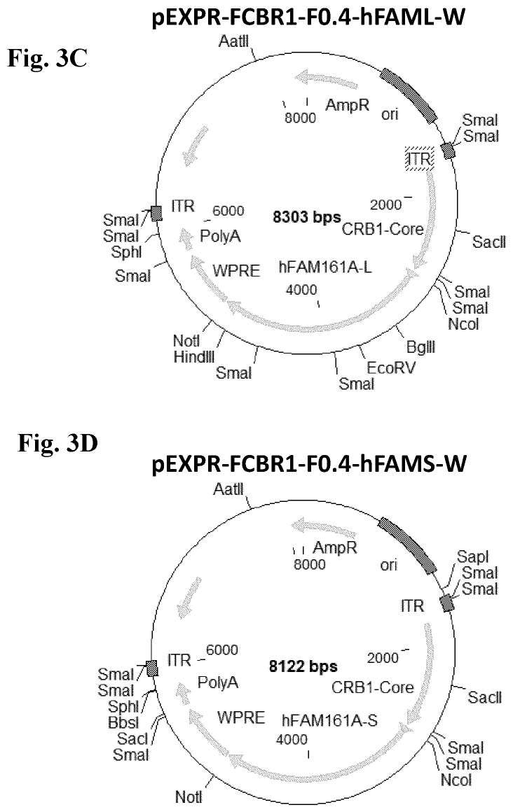

Vectors: Two different AAV2/8 vectors coding for either the long or short isoform of human FAM161A gene (hFAM161A-L (HL) or hFAM161A-S(HS)) and including the GRK1 promoter in the presence of the IRBP enhancer and the RNA stabilizer WPRE were produced as described above using plasmids (1)—SEQ ID NO: 16, (2)—SEQ ID NO: 17 (FIG. 3A, B).

Subretinal injection: Vectors were subretinally injected at a dose of 1E10 vector genome (VG) in 1 μL of PBS+0.001% pluronic into the Fam161a^{-/-} deficient mouse retina, i.e., into Fam161a^{-/-} (as described in Beryozkin et al., 2021, supra) at either post-natal day 7 or 14 (PN7 or PN14). The therapeutic vectors (1) or (2) were injected into one eye whereas the contralateral was either treated with a control vector with the GFP transgene (and the same regulatory element, i.e., AAV-GFP; 1E10 VG) or non-treated.

In another experiment, therapeutic vectors (1) and (2) were 1:1 mixed in 0.001% pluronic acid solution and injected at a total dose of 1E10 VG at PN14. Some fellow eyes were injected with AAV-GFP (dose of 1E10 VG at PN14) and were used as controls.

Electroretinogram response: Electroretinogram (ERG) were monitored at different time points after the vector injections (i.e., 1-, 2- or 3-months post injection (p.i.)) and the retinas were then analyzed by immunochemistry for FAM161A expression and assessment for retina integrity. Stimuli protocol was similar to this used in Kircher et al., 2019, Frontiers in Neurol. 10:56. In brief, animals were mice that were dark-adapted overnight and anesthetized with a mixture of ketamine (100 mg/kg) and xylazine (15 mg/kg), and pupils were dilated by topical administration of 0.5% tropicamid and then retinal activity was recorded in response to light flashes from 0.0001 to 30 cds/m2 for scotopic ERG and, after 5 min light adaptation to 1 to 30 cds/m2 for photopic ERG.

Immunohistological staining: Eyes were enucleated, fixed for 30 min in Paraformaldehyde 4% at 4° C., rinsed with PBS and incubated successively in 10%, 20% and 30% sucrose for 2 hours to overnight at 4° C. After embedding in albumin from hen egg white (Fluka, Buchs, Switzerland), 14 μm cryosection were collected. Sections were incubated at 4° C. for overnight with antibodies raised against FAM161A (Sigma HPA032119, 1:1000) and acetylated-tubulin (Sigma T7451, 1:1000) which were then detected using Goat Anti-Rabbit-Alexa Fluor® 488 and Goat Anti-Mouse-Alexa Fluor® 633 (Invitrogen, 1:2000).

Statistical analysis: Non-parametric two-tailed paired T-test (Wilcoxon ranking test) were performed using Graphpad Prism software to evaluate the significant difference of b-wave amplitudes between AAV-GFP and HL (1) or AAV-GFP and HS (2) delivery at particular time point post-injection. For some experiments, data were obtained also until 4 and 8 months p.i.

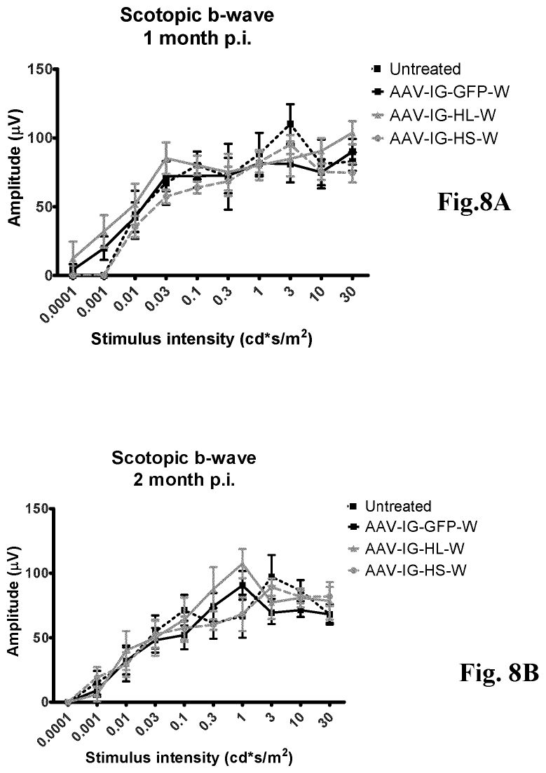

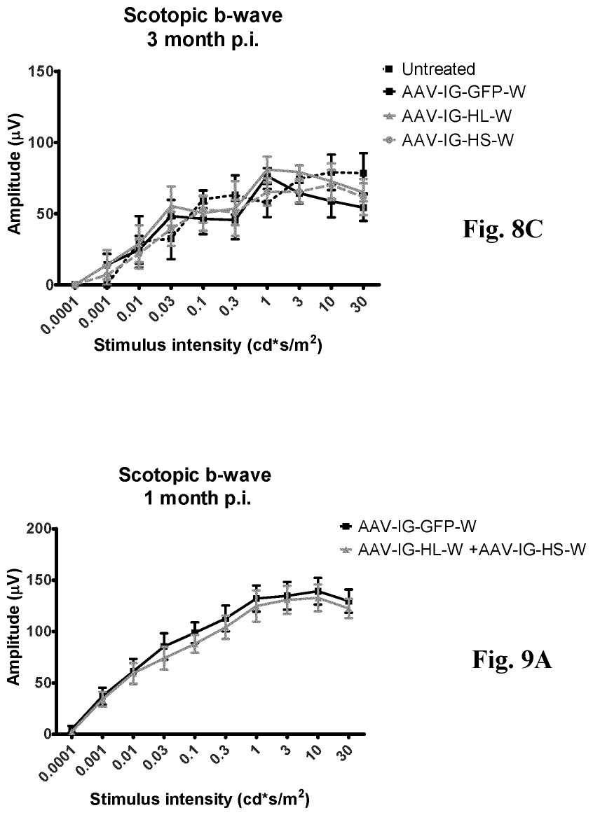

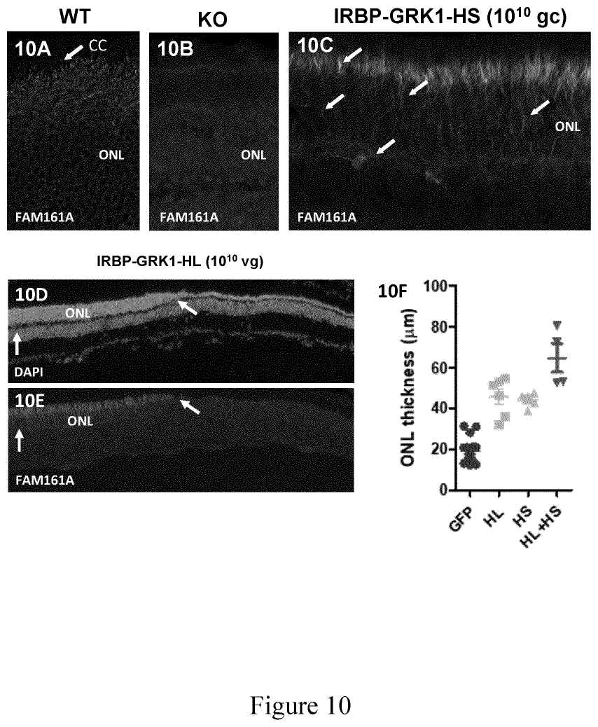

Results: In Fam161a^{-/-} mice injected with either AAV2/8-IRBP-GRK1-hFAM161A-L-WPRE (1), the AAV2/8-IRBP-GRK1-hFAM161A-S-WPRE (2) or the AAV-GFP vector, the ERG recordings at 3-month p.i. did not show marked amelioration of the retina activity of the retina treated with AAV2/8-IRBP-GRK1-hFAM161A-L-WPRE (1) or the AAV2/8-IRBP-GRK1-hFAM161A-S-WPRE (2). Nonetheless the HL vector (1) produced a small and significant ERG response protection but with high variation (+4 to +178% increase of the maximal b-wave response, p=0.0039) at 2- and 3-month p.i. in comparison to the control eye (FIG. 8). Histological examination of FAM161A expression in the treated retina 3 months p.i. revealed a strong expression of the protein in the whole cilium and the cell body with a pattern not seen in the WT retina suggesting an uncontrolled overexpression of the protein (FIG. 10). The FAM161A distribution and expression was similar to this presented in the FIG. 10 for the combined injection. These result shows that despite imperfect expression pattern, a short-term rescue can be observed with AAV2/8-IRBP-GRK1-hFAM161A-L-WPRE (1).

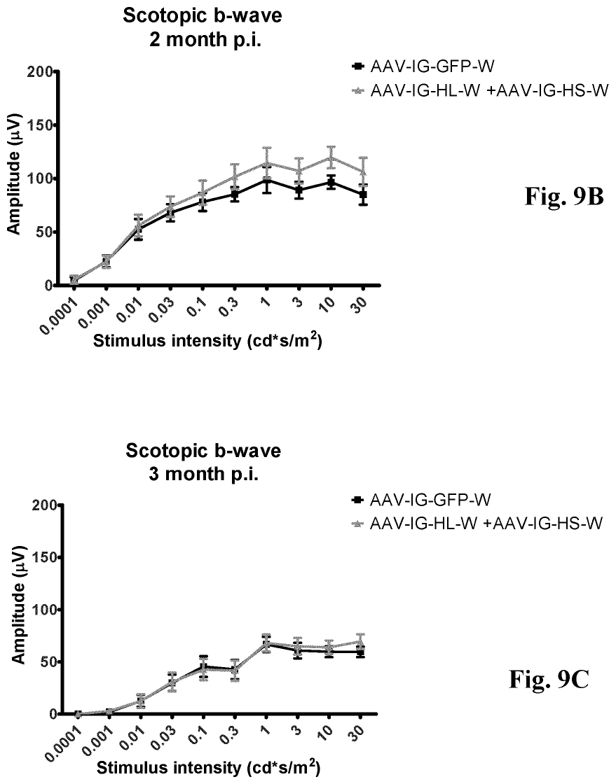

A combination of AAV2/8-IRBP-GRK1-hFAM161A-L-WPRE (1) and the AAV2/8-IRBP-GRK1-hFAM161A-S-WPRE (2) vector was also tested in the Fam161a^{-/-} retina. Control group received the AAV-GFP. At 2 months p.i., the ERG responses were significantly preserved in the treated animals, corresponding to +23.9% (p=0.0039) of the control group receiving the GFP transgene at 10 cd*s/m2 (FIG. 9B), but ERG responses were similar between groups at 1 month (FIG. 9A) and 3 months p.i. (FIG. 9C). It is hypothesized that the transient rescue observed with the mix of AAV2/8-IRBP-GRK1-hFAM161A-L-WPRE (1) and the AAV2/8-IRBP-GRK1-hFAM161A-S-WPRE (2) vector is due to the use of the human sequence in mice (see Example 4).

The subsequent histological analysis has showed that the injected area with the human short or the long isoform showed a thicker retina in comparison to non-injected region of the same eye (FIG. 10 D-F), i.e., 38.69±3.82 μm of outer nuclear thickness (ONL, outer nuclear layer) versus 17.16±1.18 μm respectively. FAM161A expression was detected in the segments and the cell body of the photoreceptors and was not restricted to CC as expected (FIG. 10C). Thus, the treatment partially reestablished a normal structure of the photoreceptor cells, efficiently protected them from degeneration, and transiently preserved their functions. However, ectopic expression of FAM161A leads to tubulin uncontrolled outgrowth affecting probably the cell function.

These vectors seem to be not the most appropriate to restore full structure and function in the Fam161a^{-/-} mice. Nonetheless, as only 58% of sequence similarity exists between mouse and human FAM161A protein sequences, one cannot rule out that the human FAM161A is not efficient to restore mouse retinal function. Indeed, experiments with vectors coding for mouse Fam161a long isoform give better rescue and support the fact that the use of FAM161A human sequences in human will be efficient.

### Example 5: Generation of Plasmids with FCBR1-F0.4 or FCBR2-F0.4

Endogenous regulatory sequence of the hFAM161A gene were used to engineer a specific photoreceptor promoter.

Material and methods: Two Crx-binding region (FCBR1 and FCBR2) and the core promoter region (F0.4) from the human gene were amplified by PCR and fused to build AAV2/8-FCBR1-F0.4-hFAM161A-L-WPRE ((10) SEQ ID NO: 25), AAV2/8-FCBR1-F0.4-hFAM161A-S-WPRE ((11) SEQ ID NO: 26), AAV2/8-FCBR2-F0.4-hFAM161A-L-WPRE ((6) SEQ ID NO: 21), AAV2/8-FCBR2-F0.4-hFAM161A-S-WPRE ((7) SEQ ID NO: 22) and their equivalent for the mouse L and L1 isoforms (8,9,12,13-SEQ ID NOs: 23, 24, 27 and 28 respectively).

Results: The maps of AAV2/8-FCBR1-F0.4-hFAM161A-L-WPRE (10) and AAV2/8-FCBR1-F0.4-hFAM161A-S-WPRE (11) are shown in FIGS. 3C and 3D respectively.

### Example 6: Generation of Vectors with FCBR1-F0.4 or FCBR2-F0.4 and FAM161A Isoforms and In Vivo Testing

Several different AAV2/8 vectors coding for either the human FAM161A gene or mouse Fam161a gene including the FCBR1-F0.4 or FCBR2-F0.4 promoter and the RNA stabilizer WPRE were compared. Subsequently, FAM161A protein expression and function in rod and cone photoreceptors was observed and compared.

Material and methods: Vectors were obtained as describe above.

Subretinal injections of the vectors were performed into the Fam161a-deficient mouse retina, i.e., Fam161a^{-/-} (as described in Beryozkin et al., 2021, supra) at post-natal day 14 to 24. Animals received either vector (6) AAV2/8-FCBR2-F0.4-hFAM161A-L-WPRE, (7) AAV2/8-FCBR2-F0.4-hFAM161A-S-WPRE, (10) AAV2/8-FCBR1-F0.4-hFAM161A-L-WPRE or (11) AAV2/8-FCBR1-F0.4-hFAM161A-S-WPRE in one eye and the AAV2/8-GFP vector in the contralateral eye.

Electroretinogram response: obtained as describe above.

FAM161A immunohistological staining: obtained as describe above.

Results: Two months post-injection, in comparison to the control eye, Fam161a″-mice injected with the vectors containing the FCBR2 regulatory element (6 or 7) showed a significant maintenance of the ERG response in 3 animals out of 11 (the maximum amplitude is +25% to +156% (P=0.0078) higher in this group compared to control eyes), whereas the other eyes had a decreased response (Table 1). In several eyes, histological examination revealed that the expression of the FAM161A is abundantly present in the photoreceptor cells, from the cilium to the bipolar synapses. In addition, several interneurons and retinal ganglion cells express long FAM161A filaments, including in the optic nerve. This abnormal pattern of FAM161A expression may contribute to a lack of restoration of photoreceptor function for the majority of the cases despite preservation of photoreceptor cells as measured by ONL thickness.

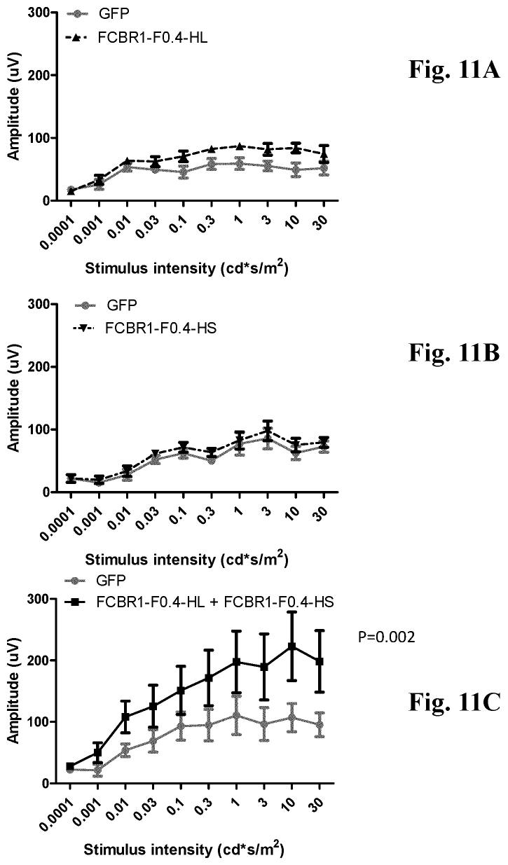

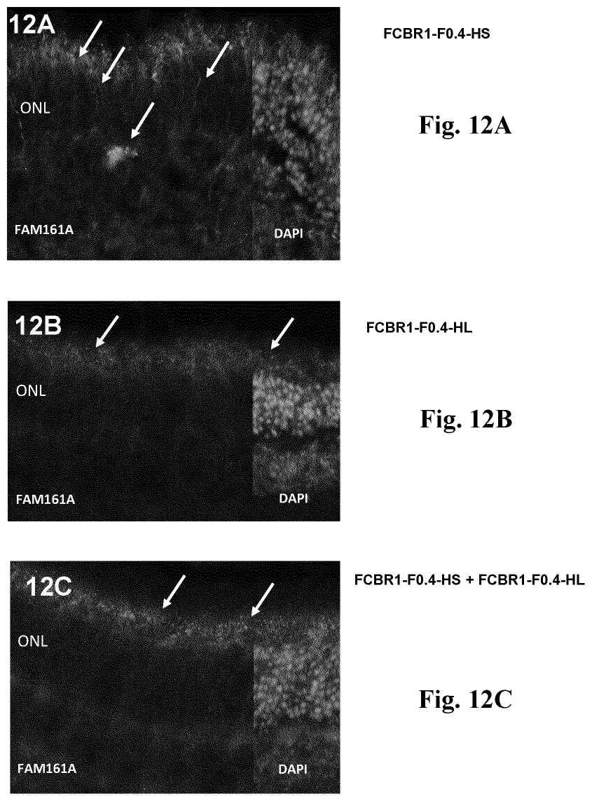

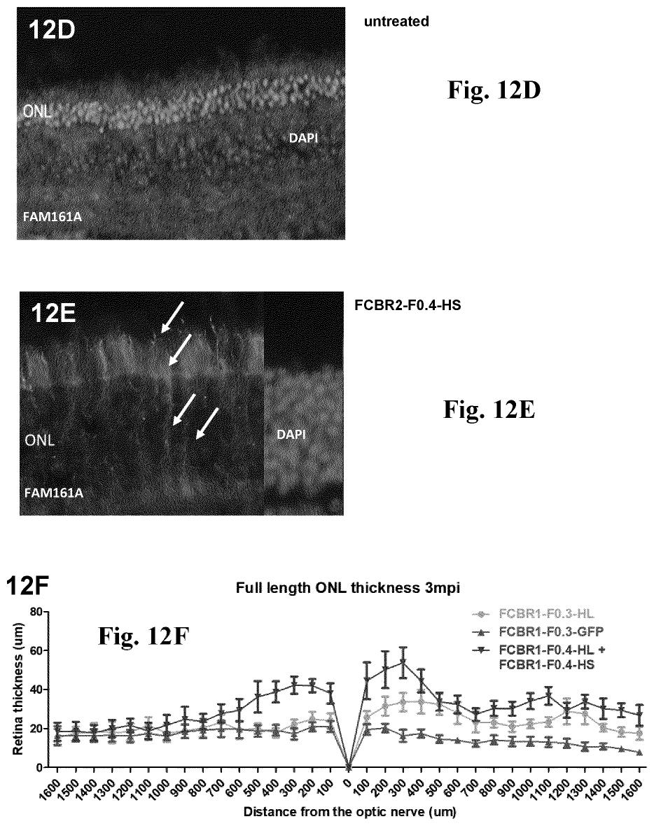

Retina from mice injected with the vector AAV2/8-FCBR1-F0.4-hFAM161A-L-WPRE (10) showed, in comparison to the AAV-GFP injected eyes, a small, but better b-wave amplitude for several stimuli 3 months post-injection (from +31 to +87% improvement, p=0.0039, Table 1, FIG. 11A). For mice treated with AAV2/8-FCBR1-F0.4-hFAM161A-S-WPRE (11), a better ERG-response response was also observed in comparison to the control eye of the same animal (from +14 to +100% improvement, p=0.0039, Table 1, FIG. 11B). Histological examination revealed a discrete expression of the FAM161A localized in the CC for the HL group (FIG. 12A) similar to a healthy retina, with some cells with few long thin filaments extending along the whole photoreceptors. The HS group showed more FAM161A ectopic expression. It was previously shown that already at PN7 the cilium in the Fami161aKO photoreceptors is altered and opened like a leak. The use of FCRB1-F0.4-vectors allow to reconstitute the cilium, when injected at PN14.

Therefore, the FCBR1-F0.4 vectors were shown to restore physiologic FAM161A expression, retinal structure, and to prolong physiological function of the photoreceptor. The FCBR2-F0.4 vectors provoked, in general for the same dose, a high expression of the FAM161A protein exceeding the connecting cilium structure and extending in ectopic cellular regions making an aberrant network. Such phenotype correlated with a diminished rate of vector efficacy in comparison to the FCRB1-F0.4 vectors. The FCBR1-F0.4-hFAM161A-L-WPRE and FCBR1-F0.4-hFAM161A-S-WPRE appear to be the most promising to modify the disease course of FAM161A to delay photoreceptor loss and to prolong retina activity.

The co-injection of AAV2/8-FCBR1-F0.4-hFAM161A-L-WPRE with AAV2/8-FCBR1-F0.4-hFAM161A-S-WPRE leads to a marked rescue of the ERG response 3 m.p.i in comparison to the control eye injected with AAV2/8-FCBR1-F0.4-GFP-WPRE vector (FIG. 11C). Indeed, an improvement of retina activity was already observed for low light stimuli indicating a preservation of rod function and the maximum b-wave amplitude (indication rod and cone responses) is 57 to 340% higher in comparison to the control eye (p=0.002).

The histological analyses revealed that the treated area was positive for FAM161A in the connecting cilium preferentially, with some photoreceptors close to the injection area also positive in the inner segment and cell body.

This series of experiments confirmed the value of AAV2/8-FCBR1-F0.4-hFAM161A-L-WPRE and AAV2/8-FCBR1-F0.4-hFAM161A-S-WPRE vectors and of the AAV2/8-IRBP-GRK1-hFAM161A-L-WPRE (1) and the AAV2/8-IRBP-GRK1-hFAM161A-S-WPRE (2) mix to both maintain retina activity and prolong retinal cell survival (FIGS. 12C and 12F).

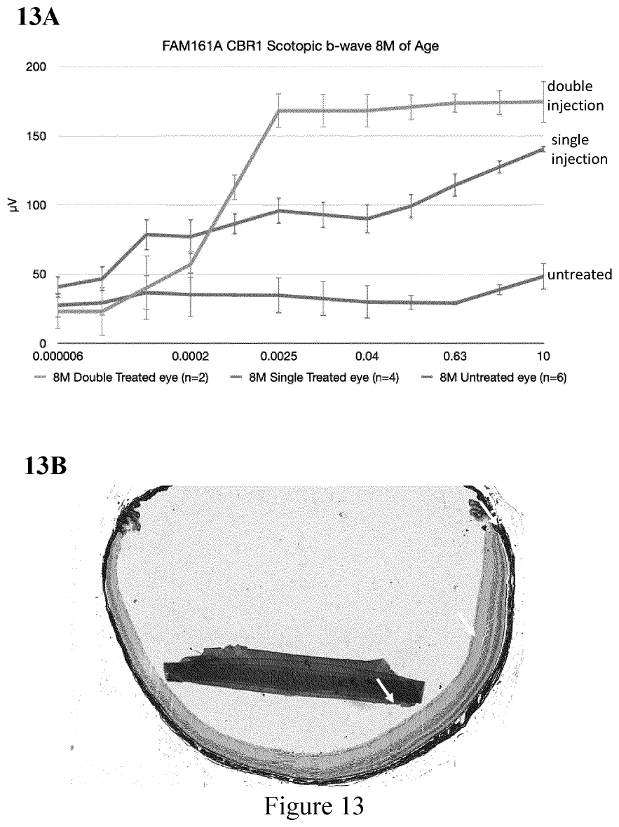

In view of the restoring retinal function effect of FCBR1-F04 vectors expressing hFAM161A isoforms, the AAV2/8-FCBR1-F0.4-mouse-L-Fam161a^{-/-} WPRE was tested in Fam161a^{-/-} mice. Analyses were made 8 mpi. At this age, the GFP control treated eye showed only a very weak responses to light stimuli (FIG. 13A), whereas eyes treated with a single or double injections showed a dramatic rescue of the retina activity (+200% to +300%). Histological analyses revealed that the treated area still contained several rows of photoreceptors, whereas the ONL contained zero to one row of photoreceptors in the non-treated area (FIG. 13B).

The vectors as detailed in Table 1 were tested in Fam161a^{-/-} mice, the protein expression and localisation as well as ERG recordings are summarised.

The AAV2/8-FCBR2-F0.4 vectors (i.e., (6), (7), (8)) were tested in the Fam161a^{-/-} mice and a strong expression was observed with a localization of the FAM161A protein exceeding the connecting cilium and overexpressed into photoreceptor cell body (Table 1). In contrast, the use of the AAV2/8-FCBR1-F0.4 vectors (i.e., (10), (11), (12), (13)) allowed a good, and specific, localization of the protein in the connecting cilium (Table 1, FIG. 12B-C).

TABLE 1
Table 1: Summary of vector efficacy. Note that vectors with the FCBR1-F0.4 sequences
give FAM161A specific localization and retina activity improvement after 3 m.p.i.
AAV-IRBP-GRK1 vector encoding for the long mouse isoform also rescued
Fam161a^{-/-} retina activity and the FAM161A protein was mainly
localized in the cilium and the inner segment.

| Promoter | Isoform | ERG | Expression/localisation (IHC) |
|---|---|---|---|
| IRBP-GRK1 | HL | 2 m.p.i.: + | + (full photoreceptor body |
| | | HL vs GFP p = 0.0117 | labelling) |
| | | 3 m.p.i.: + | |
| | | HL vs GFP p = 0.0039 | |
| | | maximum b-wave | |
| | | amplitude: +4 to +178% | |
| | HS | 2 m.p.i.: − | + (full photoreceptor body |
| | | 3 m.p.i.: − | labelling) |
| | | not significantly different | |
| | HL + HS | 2 m.p.i.: + | + (full photoreceptor body |
| | | HL + HS vs GFP p = 0.0039, | labelling) |
| | | 3 m.p.i.: − | |
| | | Non-significant | |
| | ML | 8 m.p.i.: +++ | + (inner segment and cilia |
| | | ML vs non-treated | labelling) |
| | | Scotopic: +422%, | |
| | | p.0.00037 | |
| | MS | − not significantly different | + (full photoreceptor body |
| | | | labelling) |
| | MS1 | − not significantly different | not available |
| | MS + MS1 | − not significantly different | + (full photoreceptor body |
| | | | labelling) |
| FCBR2-FCBR1-F0.4 | ML1 | +/− (p = 0.10, n = 5) | + (full photoreceptor body |
| | | | labelling) |
| FCBR1-F0.4 | GFP | | no detectable expression |
| FCBR1-F0.4 | HL | 3 m.p.i.: ++ | + (connecting cilium labelling) |
| | | HL vs GFP: p = 0.0039 | |
| | | maximum b-wave | |
| | | amplitude +31 to +87% | |
| | HS | 3 m.p.i : ++ | + (connecting cilium labelling) |
| | | HS vs GFP: p = 0.0039 | |
| | | maximum b-wave | |
| | | amplitude: +14 to +100% | |
| | HL + HS | 1 m.p.i: + | + (connecting cilium labelling) |
| | | HL + HS vs GFP p = 0.002 | |
| | | 3 m.p.i.: +++ | |
| | | HL + HS vs GFP p = 0.002 | |
| | | maximum b-wave | |
| | | amplitude: +57 to +340% | |
| | ML | 8 m.p.i.: +++ | |
| | | ML vs non-treated | |
| | | Maximum b-wave | |
| | | amplitude: +200 to +300% | |
| FCBR2-F0.4 | HS | 2 m.p.i: ++ | + (full photoreceptor body |
| | | HS vs GFP p = 0.0078 | labelling in several |
| | | maximum b-wave | photoreceptors) |
| | | amplitude: +25 to 156% | |

The literature (Langmann et al Am J Hum Genet 2010) suggested that the CBR2 sequence and the short FAM161A isoform would be the most promising combination to induce adequate expression of FAM161A, because of the activity of the Crx box-2 (CBR2) and the abundance of the short isoform in the mouse retina, but the above results contradict this hypothesis. Indeed surprisingly, in vivo vector efficacy tests show that FCBR1-F0.4 promoter drives a more adequate expression of FAM161A isoforms in the retina than FCBR2-F0.4 mimicking the endogenous expression of this gene in the photoreceptors. Indeed, the FAM161A protein is correctly located in the connecting cilium and does not extend its expression throughout the cell, as observed with the FCBR2-F0.4 promoter.

The human FAM161A gene produces two different mRNA isoforms in the retina, and the current literature does not provide any guidance if either one or both isoforms are indispensable for retinal structural preservation and functional rescue. Surprisingly, in vivo vector efficacy tests show that FCBR1-F0.4 promoter drives adequate expression of two FAM161A isoforms in the retina. Thus, both isoforms could be used separately or in combination to ensure retina activity and survival of photoreceptors of patients affected by FAM161A deficiency.

It is described that fine-tune expression of microtubule-associated proteins such as FAM161A are required to not interfere with the cellular skeleton which could then impact the general cellular transport. As photoreceptor function is highly dependent on intracellular transport of proteins to the outer segments, inadequate expression of FAM161A could negatively impact vision. Surprisingly, in vivo vector efficacy tests shows that FCBR1-F0.4 promoter drives adequate expression of two FAM161A isoforms in the photoreceptor's connecting cilium. The use of either isoform may thus serve to maintain or restructure the cilium of RP28 patients.

The efficacy of gene therapy is highly dependent on the accurate expression of the therapeutic gene. Thus, mimicking the endogenous pattern of expression is of prime importance to correctly address cellular function. This is even more pertinent for ciliary proteins which, when overexpressed, can lead to adverse cellular effect such as “paralysis” of the cytoskeleton.

It is shown that use of the FCBR1-F0.4 FAM161A promoter and its properties to correctly target FAM161A expression to the connecting cilium is of prime importance. The use of the FCBR1-F0.4 FAM161A promoter can thus likely serve for any other gene transfer aiming at restoring a normal architecture of the photoreceptor cilium.

The described gene therapy approach was validated in a mouse model of Fam161a deficiency using Fam161a sequences. With the limited homology between mouse and human protein sequences (58%), examining mouse sequences in the mouse model is more relevant for the evaluation of gene therapy success than using human sequence genes in the mouse model. However, despite this limitation, AAV-IRBP-GKR1-HL-W (1), AAV-FCBR1-F0.4-HL-W (10), AAV-FCBR1-F0.4-HS-W (11), AAV-FCBR2-F0.4-HS-W (7) and administration of mixed AAV-FCBR1-F0.4-HL-W (10) and AAV-FCBR1-F0.4-HS-W (11) showed improved photoreceptor survival and different level of increased retinal activity in the mouse model. Additionally, the success of cell survival and function maintenance of mouse Fam161a encoding AAV vectors allows to anticipate for the success of treating RP28 patients with the human FAM161A encoding AAV vectors.

Provided herein are vectors with different potential to preserve the retina integrity and/or function by combining adequate promoter and FAM161A isoform sequences. In particular, AAV2/8-IRBP-GRK1-hFAM161A-L-WPRE and AAV2/8-FCBR1-F0.4-hFAM161A-L-WPRE and the AAV2/8-FCBR1-F0.4-hFAM161A-S-WPRE have been shown as to be efficient vectors.

1. A vector comprising a FCBR1-F0.4 promotor and a nucleic acid sequence encoding a ciliary protein, or a fragment or a variant thereof.

2. A vector comprising an IRBP-GRK1 promotor and a nucleic acid sequence encoding a ciliary protein, or a fragment or a variant thereof.

3. The vector according to claim 1, wherein FCBR1-F0.4 promotor is of SEQ ID NO: 10 or SEQ ID NO: 66.

4. The vector according to claim 2, wherein IRBP-GRK1 promotor is of SEQ ID NO: 14.

5. The vector according to claim 1, wherein the nucleic acid sequence encoding the ciliary protein is selected from a long isoform of human FAM161A gene of SEQ ID NO: 1 and a short isoform of human FAM161A gene of SEQ ID NO: 2, or a fragment, or a variant thereof.

6. The vector according to claim 1, wherein a nucleic acid sequence encoding a ciliary protein is POC5 gene of SEQ ID NO: 29, NPHP4 gene of SEQ ID NO: 30, ARL6 gene of SEQ ID NO: 31, BBS1 gene of SEQ ID NO: 32, BBS2 gene of SEQ ID NO: 33, BBS9 gene of SEQ ID NO: 34, PCARE gene of SEQ ID NO: 35, CFAP418 gene of SEQ ID NO: 36, CEP164 gene of SEQ ID NO: 37 (CEP164 isoform 1) or of SEQ ID NO: 38 (CEP164 isoform 2), CEP290 gene of SEQ ID NO: 39, CLRN1 gene of SEQ ID NO: 40, IFT140 gene of SEQ ID NO: 41, IFT172 gene of SEQ ID NO: 42, IQCB1 gene of SEQ ID NO: 43, KIZ gene of SEQ ID NO: 44, LCA5 gene of SEQ ID NO: 45, MAK gene of SEQ ID NO: 46, NEK2 gene of SEQ ID NO: 47, OFD1 gene of SEQ ID NO: 48, OFD1 gene of SEQ ID NO: 49, RP1 gene of SEQ ID NO: 50, RP1L1 gene of SEQ ID NO: 51, RP2 gene of SEQ ID NO: 52, RPGR gene of SEQ ID NO: 53 (RPGR isoform C) or SEQ ID NO: 54 (RPGR isoform J), RPGRIP1 gene of SEQ ID NO: 55 (RPGRIP1 isoform 1) or SEQ ID NO: 56 (RPGRIP1 isoform 3), SPATA7 gene of SEQ ID NO: 57, TOPORS gene of SEQ ID NO: 58, TTC8 gene of SEQ ID NO: 59, USH2A gene of SEQ ID NO: 60, WDR19 gene of SEQ ID NO: 61, CFAP410 gene of SEQ ID NO: 62, or a fragment or a variant thereof.

7. The vector according to claim 1, further comprising a WPRE (woodchuck hepatitis virus post-transcriptional regulatory element) post-transcriptional regulatory element of SEQ ID NO: 15, or a fragment or a variant thereof.

8. The vector according to claim 1 that is a recombinant AAV vector that has an AAV2/8 capsid protein or an AAV2/5 capsid protein.

9. A pharmaceutical composition comprising at least one vector according to claim 1 or a combination thereof and at least one pharmaceutically acceptable vehicle.

10. (canceled)

11. A method for modifying, delaying, arresting progression, or ameliorating vision loss associated with a retinal ciliopathy comprising providing the vector according to claim 1 and administering the vector in an amount effective for modifying, delaying, arresting progression, or ameliorating of the vision loss associated with the retinal ciliopathy.

12. The method of claim 11, wherein the vector is administered to a patient in need of the treatment of the retinal ciliopathy.

13. The method of claim 12, wherein the retinal ciliopathy is FAM161A-associated retinopathy.

14. The method of claim 12, wherein the retinal ciliopathy is retinitis pigmentosa 28.

15. A method for improvement or maintenance of photoreceptor structure comprising
providing the vector according to claim 1 wherein the vector is provided in an amount effective for the improvement or the maintenance of the photoreceptor structure.

16. A method for induction of photoreceptor cilium protein stabilization comprising
providing the vector according to claim 1 wherein the vector is provided in an amount effective for the induction of the photoreceptor cilium protein stabilization.

17. The vector according to claim 2, wherein the nucleic acid sequence encoding the ciliary protein is selected from a long isoform of human FAM161A gene of SEQ ID NO: 1 and a short isoform of human FAM161A gene of SEQ ID NO: 2, or a fragment, or a variant thereof.

18. The vector according to claim 2, wherein a nucleic acid sequence encoding a ciliary protein is POC5 gene of SEQ ID NO: 29, NPHP4 gene of SEQ ID NO: 30, ARL6 gene of SEQ ID NO: 31, BBS1 gene of SEQ ID NO: 32, BBS2 gene of SEQ ID NO: 33, BBS9 gene of SEQ ID NO: 34, PCARE gene of SEQ ID NO: 35, CFAP418 gene of SEQ ID NO: 36, CEP164 gene of SEQ ID NO: 37 (CEP164 isoform 1) or of SEQ ID NO: 38 (CEP164 isoform 2), CEP290 gene of SEQ ID NO: 39, CLRN1 gene of SEQ ID NO: 40, IFT140 gene of SEQ ID NO: 41, IFT172 gene of SEQ ID NO: 42, IQCB1 gene of SEQ ID NO: 43, KIZ gene of SEQ ID NO: 44, LCA5 gene of SEQ ID NO: 45, MAK gene of SEQ ID NO: 46, NEK2 gene of SEQ ID NO: 47, OFD1 gene of SEQ ID NO: 48, OFD1 gene of SEQ ID NO: 49, RP1 gene of SEQ ID NO: 50, RP1L1 gene of SEQ ID NO: 51, RP2 gene of SEQ ID NO: 52, RPGR gene of SEQ ID NO: 53 (RPGR isoform C) or SEQ ID NO: 54 (RPGR isoform J), RPGRIP1 gene of SEQ ID NO: 55 (RPGRIP1 isoform 1) or SEQ ID NO: 56 (RPGRIP1 isoform 3), SPATA7 gene of SEQ ID NO: 57, TOPORS gene of SEQ ID NO: 58, TTC8 gene of SEQ ID NO: 59, USH2A gene of SEQ ID NO: 60, WDR19 gene of SEQ ID NO: 61, CFAP410 gene of SEQ ID NO: 62, or a fragment or a variant thereof.

19. The vector according to claim 2, further comprising a WPRE (woodchuck hepatitis virus post-transcriptional regulatory element) post-transcriptional regulatory element of SEQ ID NO: 15, or a fragment or a variant thereof.

20. The vector according to claim 2 that is a recombinant AAV vector that has an AAV2/8 capsid protein or an AAV2/5 capsid protein.

21. A method for modifying, delaying, arresting progression, or ameliorating vision loss associated with a retinal ciliopathy comprising
providing the vector according to claim 2 and administering in an amount effective for modifying, delaying, arresting progression, or ameliorating of the vision loss associated with the retinal ciliopathy.
